(19) United States
(12) Patent Application Publication
(10) Pub. No.: US 2023/0147752 A1
(43) Pub. Date: May 11, 2023

(54) INTERNAL ULTRAVIOLET THERAPY

(71) Applicant: CEDARS-SINAI MEDICAL CENTER, Los Angeles, CA (US)

(72) Inventors: Ali Rezaie, Beverly Hills, CA (US); Mark Pimentel, Los Angeles, CA (US); Gil Y. Melmed, Los Angeles, CA (US); Ruchi Mathur, Los Angeles, CA (US); Gabriela Guimaraes Sousa Leite, Porter Ranch, CA (US); Konstantin Degtyarev, River Edge, NJ (US); Larry Bischoff, Mountain Lakes, NJ (US); Kuldeep Gandhi, Parsippany, NJ (US); Michael V. Quinn, East Hanover, NJ (US); Richard Cronenberg, Mahwah, NJ (US)

(73) Assignee: CEDARS-SINAI MEDICAL CENTER, Los Angeles, CA (US)

(21) Appl. No.: 17/912,809

(22) PCT Filed: Mar. 19, 2021
(86) PCT No.: PCT/US2021/023354

§ 371 Date: Sep. 19, 2022

Related U.S. Application Data
(60) Provisional application No. 62/992,861, filed on Mar. 20, 2020.
(60) Provisional application No. 62/993,595, filed on Mar. 23, 2020.
(60) Provisional application No. 63/000,788, filed on Mar. 27, 2020.
(60) Provisional application No. 63/012,727, filed on Apr. 20, 2020.
(60) Provisional application No. 63/158,350, filed on Mar. 8, 2021.

(51) Int. Cl. A61N 5/06

(52) U.S. Cl. CPC A61N 5/0603; A61N 5/0624; A61N 2005/0604; A61N 2005/005

(57) ABSTRACT

A UV light delivery device for performing intra-corporeal ultraviolet therapy is provided. The device includes an elongated body separating a proximal end and a distal end. The device also includes a UV light source configured to be received at the receiving space and a cooling tube. In some examples, the UV light source is configured to emit light with wavelengths with a desired intensity between 320 nm and 410 nm and is utilized in conjunction with an endotracheal tube or a nasopharyngeal airway.

### CROSS REFERENCE TO RELATED APPLICATIONS

This application claims priority to U.S. Provisional Application No. 62/992,861, filed Mar. 20, 2020 titled INTERNAL ULTRAVIOLET THERAPY, U.S. Provisional Application No. 62/993,595, filed Mar. 23, 2020, titled INTERNAL ULTRAVIOLET THERAPY U.S. Provisional Application No. 63/000,788 filed Mar. 27, 2020, titled INTERNAL ULTRAVIOLET THERAPY, U.S. Provisional Application No. 63/012,727, filed Apr. 20, 2020, titled INTERNAL ULTRAVIOLET THERAPY, and U.S. Provisional Application No. 63/158,350, filed Mar. 8, 2021, titled INTERNAL ULTRAVIOLET THERAPY, the contents of all of which are incorporated herein by reference.

### FIELD OF THE DISCLOSURE

The present invention is directed to systems and methods for intra-corporeal ultraviolet therapy.

### BACKGROUND OF THE DISCLOSURE

The following description includes information that may be useful in understanding the present invention. It is not an admission that any of the information provided herein is prior art or relevant to the presently claimed invention, or that any publication specifically or implicitly referenced is prior art.

Infectious diseases, immune-mediated and inflammatory diseases continue to pose a global challenge. Despite significant strides made in the past several decades, treatment of these diseases remains suboptimal. For example, many patients may contract upper respiratory infections and pneumonia when on ventilators, which may result in death. For instance, patients that undergo ventilator treatment are intubated with an endotracheal tube (“ETT”) and may acquire an infection though the ventilation system (e.g., may acquire pneumonia). Accordingly, there is a need to reduce the rates of viral, bacterial, and other infections in the respiratory and other somatic systems of patients.

### SUMMARY OF THE DISCLOSURE

A system for performing intra-corporeal ultraviolet therapy is provided. The system includes an endotracheal tube (ETT) and a light catheter configured to be positioned within the ETT. The light catheter may include a light delivery portion comprising a set of light emitting diodes (LEDs) positioned to emit light circumferentially outward. Further, the light catheter may include a cooling tube comprising at least one opening. The light catheter may further include an ETT connector configured to connect to the ETT.

The set of LEDs may be positioned around the cooling tube such that a portion of each LED in the set of LEDs is in direct contact with the cooling tube. Further, within the cooling tube, a coolant gas may flow in a first direction towards the at least one opening and exit via the at least one opening, and flow backwards within the light catheter in a second direction opposite to the first direction.

In some examples, each LED in the set of LEDs may include a heat sink. In further examples, the heat sink may comprise one or more copper plates.

The set of LEDs emit peak wavelengths in the 340-349 nm range. In some examples, the peak wavelength may be in a range from 343 nm to 345 nm.

In some examples, the ETT connector comprises a flap valve. The system may further comprise a compressor system that includes one or more processors, an air compressor, and a dual-type connector comprising one or more air connectors and an electrical connector.

In some examples, the system further comprises an umbilical tube comprising at least one air passageway, one or more electrical conductors, a light catheter connector configured to connect to the light catheter; and a compressor connector configured to connect to the compressor system.

Also disclosed is a method of deploying the light catheter in the system for performing intra-corporeal ultraviolet therapy. The method includes connecting the ETT connector to the ETT; deploying the light catheter into the ETT by advancing the light catheter through the flap valve. Further, the method may include providing instructions to the controller to energize the set of LEDs; and energizing the air compressor to pump air through the air passageway into the cooling tube and out of the at least one opening.

Further, a thermistor may be in thermal contact with the light delivery portion, and responsive to an indication of a temperature from the thermistor, a flow rate of the coolant flow may be adjusted and/or power supplied to the set of LEDs may be adjusted.

Also disclosed herein is a method of treating a patient with a respiratory infection. The method may include intubating the patient with an ETT, the ETT coupled to a ventilator. Further, a light catheter may be connected to the ETT via an ETT connector. The light catheter includes a plurality of LEDs and a cooling channel within the light catheter. The plurality of LEDs may radiate UV-A light outwardly from the light catheter along a substantial length of the light catheter from the set of LEDs to treat an infection in the patient while ventilating the patient.

In one example, the light catheter may be advanced through the ETT connector such that a desired length of the light catheter is positioned within the ETT. Upon advancing the light catheter into the ETT, a control unit may provide an indication to the light catheter to electrically power the set of LEDs and/or a coolant flow may be activated so as to flow a coolant through the coolant tube. The coolant may exit through at least one opening toward a proximal sealed end of the light catheter (opposite a distal end connected to the ETT) and push backwards flowing along a length of the set of LEDs thereby cooling the LEDs. The warmed air may then return via a warm air tubing to the control unit or expelled to atmosphere.

Additional features and advantages of the disclosure will be set forth in the description that follows, and in part, will be obvious from the description; or can be learned by practice of the principles disclosed herein. The features and advantages of the disclosure can be realized and obtained by means of the instruments and combinations particularly pointed out in the appended claims. These and other features of the disclosure will become fully apparent from the following description and appended claims, or can be learned by the practice of the principles set forth herein.

### BRIEF DESCRIPTION OF THE DRAWINGS

In order to describe the manner in which the above-recited disclosure and its advantages and features can be obtained, a more particular description of the principles described above will be rendered by reference to specific examples illustrated in the appended drawings. These drawings depict only example aspects of the disclosure, and are therefore not to be considered as limiting of its scope. These principles are described and explained with additional specificity and detail through the use of the following drawings:

FIG. 1A illustrates a cross sectional view of an exemplary UV emitting device inserted into a colon of a patient, in accordance with the principles of the present disclosure;

FIG. 1B illustrates a cross sectional view of the exemplary UV emitting device inserted into a vagina of a patient, in accordance with the principles of the present disclosure;

FIG. 1C illustrates a cross sectional view of the exemplary UV emitting device inserted into a trachea of a patient, in accordance with the principles of the present disclosure;

FIG. 1D illustrates a cross sectional view of the exemplary UV emitting device inserted into a nasopharynx of a patient, in accordance with the principles of the present disclosure;

FIG. 1E illustrates a front view of the exemplary UV emitting device inserted into a trachea of a patient, in accordance with an embodiment of the present disclosure;

FIG. 1F illustrates an enlarged portion of FIG. 1E.

FIG. 2 illustrates a schematic view of an exemplary UV emitting device incorporating LEDs, in accordance with the principles of the present disclosure;

FIG. 3 illustrates a schematic view of an exemplary UV emitting device incorporating a cold cathode, in accordance with the principles of the present disclosure;

FIG. 4 illustrates an exemplary schematic of the UV spectrum, in accordance with the principles of the present disclosure;

FIG. 5 illustrates a cross sectional view of the exemplary UV emitting device inserted into the rectum and sigmoid of a patient, in accordance with the principles of the present disclosure;

FIG. 6 illustrates a cross sectional view of the exemplary UV emitting device inserted into the colon of a patient, in accordance with the principles of the present disclosure;

FIG. 7 illustrates a cross sectional view of UV emitting device inserted in the esophagus and stomach of a patient, in accordance with the principles of the present disclosure;

FIG. 8 illustrates a cross sectional view of the exemplary UV emitting devices traversing the digestive system of a patient, in accordance with the principles of the present disclosure;

FIG. 9 illustrates a side view of an exemplary light source attachment, in accordance with the principles of the present disclosure;

FIG. 10 illustrates an exemplary UV emitting device, in accordance with the principles of the present disclosure;

FIG. 11 illustrates an exemplary Foley catheter incorporating the exemplary UV emitting device, in accordance with the principles of the present disclosure;

FIG. 12A illustrates a growth curve of E. coli when implementing the exemplary UV emitting device of the present disclosure;

FIG. 12B illustrates a growth curve of E. coli when implementing the exemplary UV emitting device of the present disclosure;

FIG. 13 illustrates an exemplary UV emitting device implemented in the colon of a mouse, in accordance with the principles of the present disclosure;

FIGS. 14A and 14B illustrate an exemplary UV emitting device of the present disclosure inserted into the vaginal canal of a rat, in accordance with the principles of the present disclosure;

FIG. 15A illustrates a growth curve of liquid culture containing E. coli when implementing the exemplary UV emitting device of the present disclosure;

FIG. 15B illustrates an exemplary UV emitting device of the present disclosure implemented on a liquid culture containing E. coli;

FIG. 16 illustrates a growth curve of liquid culture containing E. coli when implementing an exemplary UV emitting device of the present disclosure;

FIGS. 17A and 17B illustrate growth curves of liquid culture containing E. coli when implementing an exemplary UV emitting device of the present disclosure;

FIG. 18 illustrates a growth curve of liquid culture containing E. coli when implementing an exemplary UV emitting device of the present disclosure;

FIG. 19 illustrates a growth curve of liquid culture containing E. coli when implementing an exemplary UV emitting device of the present disclosure;

FIG. 20 illustrates a growth curve of liquid culture containing E. coli when implementing an exemplary UV emitting device of the present disclosure;

FIGS. 21A and 21B illustrate a growth curve of liquid culture containing E. coli when implementing an exemplary UV emitting device of the present disclosure;

FIG. 22 illustrates an exemplary UV emitting device, in accordance with an embodiment of the present disclosure;

FIG. 23 illustrates the exemplary UV emitting device of FIG. 22 mounted to a gripping element 200, in accordance with an embodiment of the present disclosure;

FIG. 24 illustrates an exemplary UV emitting device, in accordance with an embodiment of the present disclosure;

FIG. 25 illustrates an exemplary UV emitting device, in accordance with an embodiment of the present disclosure;

FIG. 26 illustrates an exemplary UV emitting device in accordance with an embodiment of the present disclosure;

FIG. 27 illustrates an exemplary UV emitting device, in accordance with an embodiment of the present disclosure;

FIG. 28 illustrates an exemplary UV emitting device in accordance with an embodiment of the present disclosure;

FIG. 29 illustrates an exemplary UV emitting device in accordance with an embodiment of the present disclosure; and

FIG. 30 illustrates an exemplary process for performing intra-corporeal ultraviolet therapy, in accordance with an embodiment of the present disclosure.

FIG. 31 illustrates an exemplary process for performing intra-corporeal ultraviolet therapy in connection with an ETT, in accordance with an embodiment of the present disclosure.

FIG. 32 shows a schematic of a Chip on Board (COB) mini bar utilized as a UV LED light source, in accordance with an embodiment of the present disclosure;

FIG. 33 shows a schematic of an example UV light catheter comprising one or more COB mini bars included within an outer tube, in accordance with an embodiment of the present disclosure;

FIG. 34A shows a schematic of a fiber optic system coupled to a UV LED light source, in accordance with an embodiment of the present disclosure;

FIGS. 34B and 34C show schematic illustrations of multiple UV LED light sources for implantation with the fiber optic system, in accordance with an embodiment of the present disclosure;

FIG. 35A shows a flat configuration and a tubular configuration of a flexible printed circuit board (PCB) utilized in conjunction with one or more UV LED light sources, in accordance with an embodiment of the present disclosure;

FIG. 35B shows an example UV light catheter comprising one or more flexible PCBs, in accordance with an embodiment of the present disclosure;

FIG. 35C shows an example heat sink implemented in a UV light catheter, such as UV light catheter at FIG. 35B, in accordance with an embodiment of the present disclosure;

FIG. 36A shows an example UV light catheter comprising a plurality of LEDs and a plurality of linear reflectors, in accordance with an embodiment of the present disclosure;

FIG. 36B shows an example configuration of a plurality of LEDs and a plurality of linear reflectors, in accordance with an embodiment of the present disclosure;

FIG. 36C shows an example heat sink implemented in a UV light catheter, such as UV light catheter at FIG. 36B, in accordance with an embodiment of the present disclosure;

FIG. 36D illustrates example light distribution in an example UV LED light source including a plurality of LEDs and a plurality of linear reflectors, in accordance with an embodiment of the present disclosure;

FIG. 37 shows an example beam angle of a UV LED light source, in accordance with an embodiment of the present disclosure;

FIG. 38 shows a block diagram illustrating an example safety assessment process using human cell lines, in accordance with an embodiment of the present disclosure;

FIGS. 39 and 40 show bar graphs illustrating cell growth of HeLa cells and Alveolar cells respectively following exposure to UVA light using an exemplary system according to the present disclosure;

FIG. 41 shows a block diagram illustrating an example safety assessment process on HeLa cell lines at a higher UVA dosage, in accordance with an embodiment of the present disclosure;

FIG. 42 shows a bar graph illustrating cell growth of HeLa cells following exposure to UVA light at a higher dosage using an exemplary system according to the present disclosure;

FIG. 43 shows block diagrams illustrating example processes for assessment of UVA pre-treatment of fluorescence-tagged Coxsackie virus prior to infection of HeLa cell lines, in accordance with an embodiment of the present disclosure;

FIGS. 44A and 44B shows fluorescence images of HeLa cells transfected with fluorescence-tagged Coxsackie virus, the fluorescence-tagged Coxsackie virus pre-treated with UVA prior to transfection of HeLa cells, using an exemplary system according to the present disclosure;

FIG. 45 shows a block diagram illustrating an example assessment of HeLa cell lines pre-treated with UVA prior to transfection with Coxsackie virus, in accordance with an embodiment of the present disclosure;

FIGS. 46A and 46B show example fluorescence images of HeLa cells transfected with fluorescence-tagged Coxsackie virus, the HeLa pre-treated with UVA prior to transfection with Coxsackie virus, using an exemplary system according to the present disclosure;

FIG. 47 shows a block diagram illustrating an example process for evaluating effect of UVA light treatment on Coxsackie virus transfected alveolar cells.

FIG. 48 shows fluorescence images of alveolar cells transfected with Coxsackie virus and effect of UVA treatment on transfected alveolar cells, in accordance with an embodiment of the present disclosure;

FIG. 49 shows a block diagram illustrating an example process for evaluating effect of UVA light treatment on Coxsackie virus transfected HeLa cells.

FIG. 50 shows a bar graph illustrating effect of UVA treatment on survival of Coxsackie virus transfected HeLa cells, in accordance with an embodiment of the present disclosure;

FIG. 51 shows phase contrast images of UVA treated and untreated ciliated tracheal epithelial cells (HTeC) transfected with Coronavirus 229E, in accordance with an embodiment of the present disclosure;

FIGS. 52, 53, and 54 show bar graphs illustrating viability of ciliated tracheal epithelial cells depending on transfection with coronavirus 229E and treatment with UVA light, in accordance with an embodiment of the present disclosure;

FIG. 55 illustrates a table showing the intensities and exposure durations of UVA light applied to bacterial cultures in one example.

FIG. 56 illustrates a table showing bacterial counts over time during UV light exposure in one example.

FIG. 57 illustrates growth curve showing bacterial counts over time during UV light exposure using an exemplary system according to the present disclosure.

FIG. 58A illustrates images of petri dishes containing bacteria exposed to UV light over time compared to control.

FIGS. 58B-56E illustrate growth curves showing E. coli bacterial counts over time exposed to various intensities of UV light using an exemplary system according to the present disclosure.

FIG. 58F (intentionally omitted).

FIGS. 58G-58J illustrate growth curves showing P. aeruginosa bacterial counts over time exposed to various intensities of UV light using exemplary systems according to the present disclosure.

FIGS. 58K-58L illustrate growth curves comparing the logarithmic reduction at various intensities at 20 minutes and 40 minutes respectively using exemplary systems according to the present disclosure.

FIG. 58M illustrates growth curves showing the reduction of a E. coli colony diameter at various intensities and treatment times using an exemplary system according to the present disclosure.

FIG. 58N illustrates growth curves showing the reduction of a P. aeruginosa colony diameter at various intensities and treatment times using an exemplary system according to the present disclosure.

FIG. 59A illustrates a bar graph showing cell growth during exposure to UVA light using an exemplary system according to the present disclosure.

FIG. 59B illustrates a bar graph showing cell growth during exposure to UVA light using an exemplary system according to the present disclosure.

FIG. 59C illustrates a bar graph showing cell growth during exposure to UVA light using an exemplary system according to the present disclosure.

FIG. 59D illustrates a bar graph showing the absence of DNA damage to cells during exposure to UVA light using an exemplary system according to the present disclosure.

FIG. 59E illustrates a bar graph showing lack of DNA damage to cells during exposure to UVA light using an exemplary system according to the present disclosure.

FIG. 59F illustrates a bar graph showing lack of DNA damage to cells during exposure to UVA light using an exemplary system according to the present disclosure.

FIG. 60 shows fluorescence images illustrating effects of UVA exposure on group B Coxsackie virus pre-treated with UVA, using an exemplary system according to the present disclosure.

FIG. 61 shows fluorescence images illustrating effects of narrow band (NB)-UVA exposure on HeLa cells transfected with group B coxsackievirus, using an exemplary system according to the present disclosure;

FIG. 62 illustrates a bar graph showing cell growth transfected with a virus during exposure to UV light using an exemplary system according to the present disclosure;

FIG. 63 illustrates a bar graph showing the cell counts of transfected cells after 72 hours of UV light application compared to controls using an exemplary system according to the present disclosure;

FIG. 64 shows a schematic overview of a light treatment system, in accordance with an embodiment of the present disclosure;

FIG. 65 shows a schematic of a UV light catheter, in accordance with an embodiment of the present disclosure;

FIG. 66 shows a schematic of an enlarged portion of the UV light catheter of FIG. 65;

FIG. 67 shows a schematic of UV light catheter of FIG. 65 including one or more depth markings, in accordance with an embodiment of the present disclosure

FIG. 68 shows a schematic of the UV light catheter of FIG. 65 in a deployed configuration within an ETT, in accordance with an embodiment of the present disclosure;

FIG. 69 shows a schematic of an enlarged portion of the UV light catheter of FIG. 68;

FIG. 70 shows a schematic of a light emitting portion of a UV light catheter, in accordance with an embodiment of the present disclosure;

FIG. 71 shows a schematic of a beam angle of a UV-LED used in a UV light catheter, in accordance with an embodiment of the present disclosure;

FIG. 72 shows a schematic of a light emitting portion of a UV light catheter, in accordance with an embodiment of the present disclosure;

FIG. 73 shows table depicting baseline characteristics of subjects in an in-human study of UVA treatment performed using an exemplary system according to the present disclosure;

FIG. 74 shows a graph illustrating change in endotracheal SARS-COV-2 loads over a course of UVA treatment in an in-human study using an exemplary system according to the present disclosure;

FIG. 75 shows a table depicting corresponding viral loads in FIG. 74 at baseline (day 0), day 5, and day 6 of UVA treatment; and

FIG. 76 shows a summary of timeline and key events for subjects in the in-human study of FIGS. 73-74.

### DETAILED DESCRIPTION

### Definitions

Unless defined otherwise, technical and scientific terms used herein have the same meaning as commonly understood by one of ordinary skill in the art to which this invention belongs. Szycher's Dictionary of Medical Devices CRC Press, 1995, may provide useful guidance to many of the terms and phrases used herein. One skilled in the art will recognize many methods and materials similar or equivalent to those described herein, which could be used in the practice of the present invention. Indeed, the present invention is in no way limited to the methods and materials specifically described. For example, the Figures primarily illustrate the present invention in the gastrointestinal tract, but as indicated throughout, the disclosed systems and methods can be used for other applications.

In some embodiments, properties such as dimensions, shapes, relative positions, and so forth, used to describe and claim certain embodiments of the invention are to be understood as being modified by the term “about.”

As used herein, “ETT” refers to an endotracheal tube, which is a flexible tube placed through the mouth of a patient into the trachea to assist a patient in breathing while connected to a ventilator.

As used herein, “NPA” refers to a nasopharyngeal airway, which is a flexible tube placed through the nasal passageway and ending at the base of the tongue to assist in maintaining an open airway.

As used herein, the term “LED” refers to a light emitting diode that is a semiconductor light source that emits light across various visible and non-visible light spectrums. LEDs typically have an emission spectrum that includes a set of wavelengths that vary in intensity over their emission spectrum range, and typically follow a bell or similar shaped intensity curve over that wavelength range. Specific LEDs are typically described using their wavelength of peak emission intensity, or the wavelength at which the LED emits its highest intensity of radiation.

Accordingly, LEDs typically emit light across a range of wavelengths, and specific LEDs may also be described using the range of wavelengths it emits over a threshold intensity (in some examples, a percentage of the LEDs maximum intensity). For instance, a given LED may emit light with at least 10% of its maximum emission intensity only between the wavelengths of 335 nm and 345 nm. Below 335 nm and above 345 nm, that LED's intensity of emission may be less than 10% of that LED's peak intensity emission wavelength (“peak wavelength” herein), and in some cases too low to be therapeutically relevant. Therefore, for many treatment applications, only the wavelengths between 335 nm and 345 nm would have an impact on treatment for that specific LED.

Accordingly, the range of wavelengths described herein may be the range of wavelengths that is therapeutically effective or significant for a particular treatment application, duration, and intensity of emission delivered by the LED to the treatment site (or based on power of emission emitted by the LED). In some examples, the range of wavelengths may be the range of wavelengths emitted by the LEDs that have an intensity that is at least 1, 2, 3, 4, 5, 6, 7, 8, 9, 10, 11, 12, 13, 14, 15, 16, 17, 18, 19, or 20% of the peak emission intensity.

Accordingly, disclosed herein are emission spectrum ranges for various LED light sources which correspond to the ranges for which the LED emits a threshold intensity percentage of its maximum intensity. Examples of various LED spectrum emission ranges and peak intensity wavelengths of emission of commercially available LEDs are described in Filippo, et al, “LEDs: Sources and Intrinsically Bandwidth-Limited Detectors,” the content of which is incorporated by reference in its entirety.

Various examples of the invention will now be described. The following description provides specific details for a thorough understanding and enabling description of these examples. One skilled in the relevant art will understand, however, that the invention may be practiced without many of these details. Likewise, one skilled in the relevant art will also understand that the invention can include many other obvious features not described in detail herein. Additionally, some well-known structures or functions may not be shown or described in detail below, so as to avoid unnecessarily obscuring the relevant description.

The terminology used below is to be interpreted in its broadest reasonable manner, even though it is being used in conjunction with a detailed description of certain specific examples of the invention. Indeed, certain terms may even be emphasized below; however, any terminology intended to be interpreted in any restricted manner will be overtly and specifically defined as such in this Detailed Description section.

While this specification contains many specific implementation details, these should not be construed as limitations on the scope of any inventions or of what may be claimed, but rather as descriptions of features specific to particular implementations of particular inventions. Certain features that are described in this specification in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations may be depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

### Overview

While UV light in the UVA and UVB range has traditionally been used to treat dermatologic disorders, it has not been developed for broader infection or inflammation treatment inside the human body. The present disclosure describes a system for emission of therapeutic doses of UV light via a catheter, capsule, endoscope, tube, or port that can be used to manage internal infections and inflammatory conditions inside a patient. The UV light source disclosed herein is intended to provide a safe and effective alternative to antibiotics and anti-inflammatory/immunosuppressant drugs to various internal canals of a patient (e.g. colon, vagina, trachea).

In some examples, only UVA light or only UVB light may be emitted for certain indications and treatments. For instance, a UV light source may have wavelengths centered around 335 nm, 340 nm, or 345 nm or nearby ranges as disclosed herein. In other embodiments, the UV light sources may emit wavelengths between 320 nm-410 nm, and/or have a peak intensity of emission within that range. It should be understood that various wavelengths can be provided using the systems and methods. In some examples, the wavelength range provided may be the highest wavelength possible that is therapeutically effective in a certain intensity and duration of application.

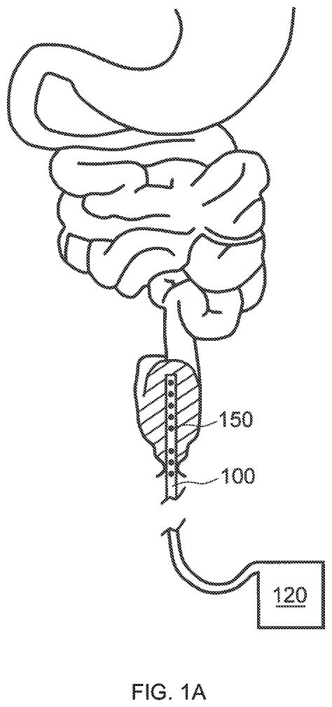

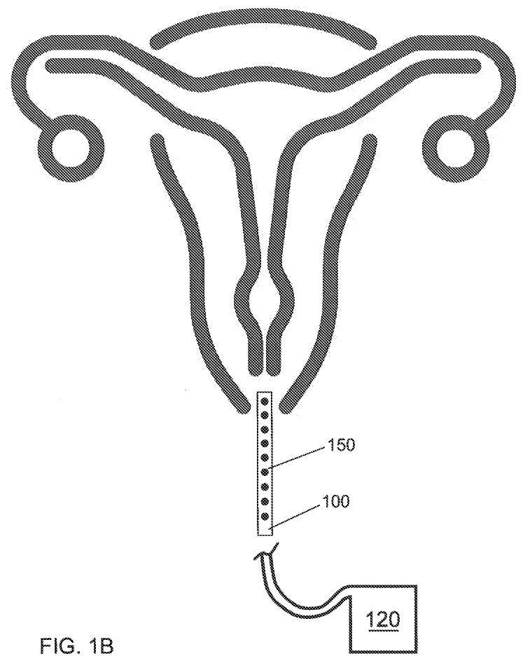

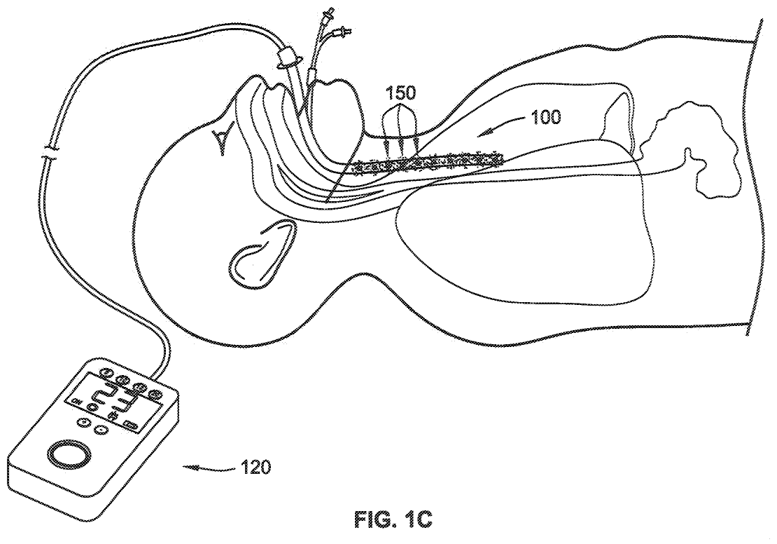

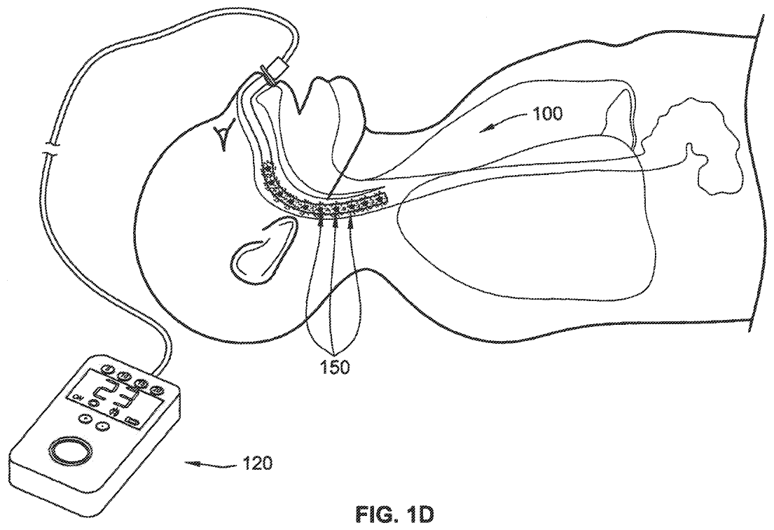

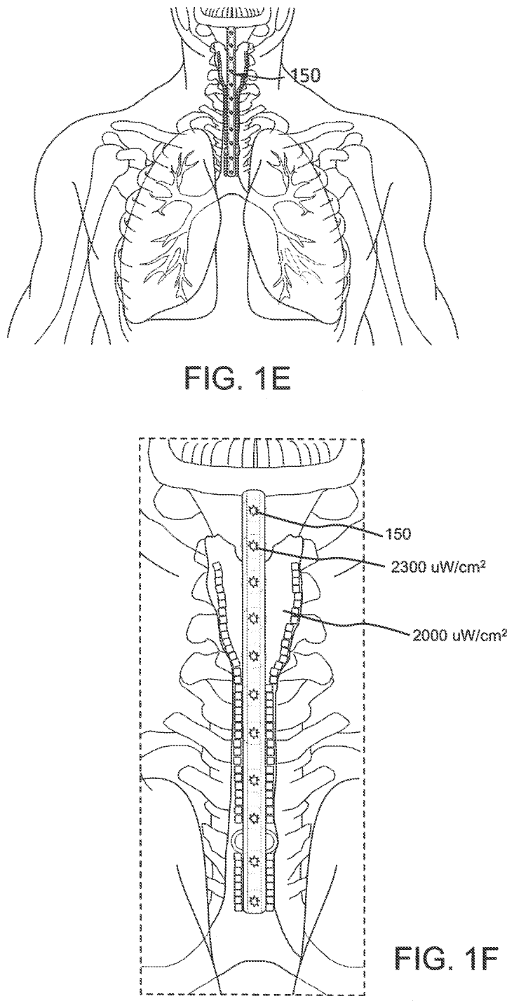

FIG. 1A illustrates an example of a UV light administrative system that includes a delivery tube 100 and several UV light sources 150, and a power source 120 to power the system. Accordingly, as illustrated, a caregiver (e.g., physician) can navigate the delivery tube 100 to a colon of a patient. Once navigated to the intended treatment target of the patient, the power source 120 may be energized to emit UV light from the light sources 150 into the therapeutic target (e.g. colon).

FIG. 1B illustrates an example of a UV light administrative system that includes a delivery tube 100, several UV light sources 150, and a power source 120. Accordingly, a caregiver (e.g., physician) can navigate the delivery tube 100 to a vagina of a patient. Once navigated to the vagina of the patient, the delivery tube 100 can be energized by the power source 120 to emit therapeutic light (e.g., UV light) into the vaginal canal. The UV light source disclosed herein is intended to provide a safe and effective alternative to antibiotics and anti-inflammatory/immunosuppressant drugs to the colon region and/or the vagina region.

FIG. 1C illustrates an example of a UV light administrative system that includes a delivery tube 100, UV light sources 150, a power source 120, and a control system. The control system provides power and controls the duration and/or intensity of treatment. Accordingly, as illustrated, a caregiver (e.g., physician) can navigate the delivery tube 100 to a trachea of a patient during ventilation. Once navigated to the trachea of the patient, the power source 120 can be energized so that it delivers power to the light sources 150 through the delivery tube 100 (e.g. wired connections) to emit therapeutic light (e.g., UV light) into the trachea and/or other respiratory canals.

For instance, systems and methods have been developed to provide internal ultraviolet therapy in conjunction with an endotracheal tube (ETT) as disclosed herein. Accordingly, the delivery tube 100 may be navigated inside an ETT during ventilation of a patient. In other examples, the delivery tube 100 may be connected to or built into an ETT, or an ETT may have light sources 150 incorporated into the ETT. Accordingly, the light sources 150 may be positioned within the tube 150 and/or ETT so that the UV light sources 150 radiate the respiratory tissue in the tracheal airways surrounding the ETT.

FIG. 1D illustrates an example of a UV light administrative system that includes a delivery tube 100, UV light sources 150, a power source 120, and a control system. The control system provides power and controls the duration and/or intensity of treatment. Accordingly, as illustrated, a caregiver (e.g., physician) can navigate the delivery tube 100 to the nasopharynx of a patient. Once navigated to the nasopharynx of the patient, the power source 120 can be energized so that it delivers power to the light sources 150 through the delivery tube 100 (e.g. wired connections) to emit therapeutic light (e.g., UV light) into the nasopharynx and/or other respiratory canals.

For instance, systems and methods have been developed to provide internal ultraviolet therapy in conjunction with a nasopharynx airway (NPA) as disclosed herein. Accordingly, the delivery tube 100 may be navigated inside an NPA of a patient. In other examples, the delivery tube 100 may be connected to or built into an NPS, or an NPA may have light sources 150 incorporated into the NPA. Accordingly, the light sources 150 may be positioned within the tube 150 and/or NPA so that the UV light sources 150 radiate the respiratory tissue in the nasopharynx surrounding the NPA.

FIG. 1E shows a front view of the UV light administrative system that includes a plurality of light sources 150 within a trachea of a patient. FIG. 1F is an enlarged portion of FIG. 1E depicting change in UV light intensity with increasing distance from the light sources 150. Accordingly, in some examples, power to each LED may be individually controlled depending on a distance to a tissue to be irradiated.

### Delivery Systems

A delivery tube/rod 100 for delivering therapeutic UV light to various portions inside a body is provided. The delivery tube/rod can include at least one UV light source 150. The delivery tube/rod 100 can be a catheter, endoscope, capsule (for swallowing or suppository), or any other medical device configured to receive a UV light source 150.

In some examples, the UV delivery tube 100 may be configured as a catheter, and navigated inside of an ETT or an NPA during respiratory or other therapy of a patient. In some embodiments, the UV delivery tube/rod 100 is configured as an endoscope, which is inserted rectally or orally, and navigated to the appropriate regions to deliver anti-inflammatory or other therapeutic doses of UV light. In another embodiment, the UV delivery tube/rod 100 can be configured as a catheter, which is inserted into arteries, urethra, vagina and urinary tract, ear canal, airways etc. In yet another embodiment, the UV delivery tube/rod 100 is configured as an indwelling urinary catheter, which is inserted into a patient's bladder. In some embodiments, an inflatable balloon catheter can include the UV light source 150 to emit UV light inside internal organs with passageways, such as, e.g., the vagina, rectum, gastroesophageal junction, stomach, biliary tract, or other suitable passageways. In some embodiments, the UV light source 150 can be configured as a caregiver's glove. This configuration may assist with emitting UV light into a patient's orifice (e.g., a mouth, a rectum, a vagina, or others) for shorter duration treatments.

In some embodiments, UV light sources 150 are permanently mounted onto the delivery tube/rod 100. In other embodiments, the delivery tube/rod 100 is configured such that the UV light sources 150 are configurable, and able to be mounted and removed at a physician's preference. The delivery tube/rod 100 can include a hollow interior to allow for electrical connections to the UV light sources 150. In alternative embodiments, the UV light sources 150 may be wireless, and able to couple to the delivery tube/rod 100.

### Light Sources

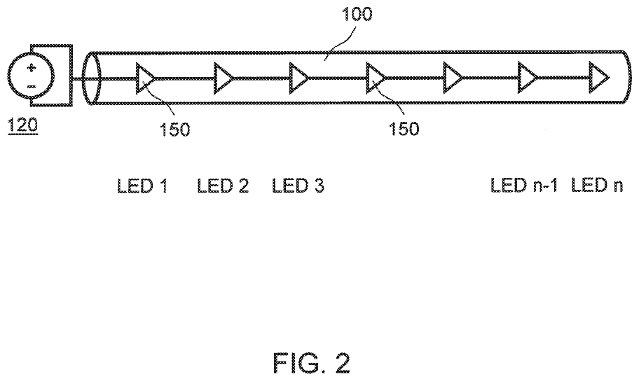

Depending on the delivery tube 100 or other delivery device, various light sources 150 may be utilized that are capable of emitting UV light. For instance, FIG. 2 illustrates an embodiment of a flexible delivery tube 100 (e.g., catheter, endoscope, or the like) that includes a string of LED light sources 150 that are distributed along the tube 100. In other examples, other suitable light sources 150 capable of emitting UV light may be utilized. Each of the light sources 150 are attached together with electrical connections and connected to a power supply 120. LED light sources 150 may be advantageous, since their small size and low power requirements enable them to be placed along the delivery tube 100.

Accordingly, if the light sources 150 are placed along the delivery tube 100, the light sources 150 may deliver a UV light to a large delivery area inside the patient. Accordingly, the therapeutic target area may be relatively large, to treat inflammatory diseases that may affect a large portion of the colon.

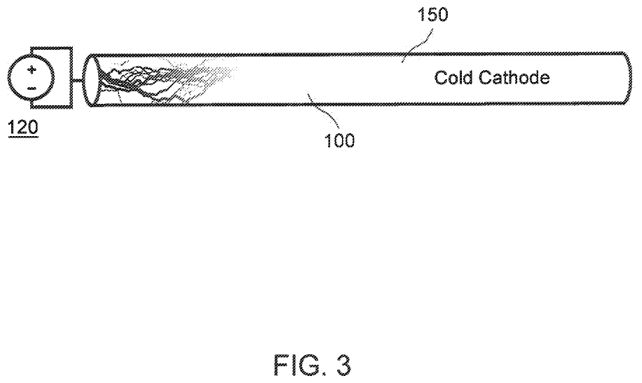

FIG. 3 illustrates an example of a delivery tube 100 that utilizes a cold cathode based light source 150 that is connected to a power supply 120. In this embodiment, the cold cathode light source 150 delivers light through a transparent, flexible delivery tube 100. This embodiment may include an inert gas that fills the delivery tube (or a vacuum tube) 100. The delivery tube 100 may include, e.g., a cold cathode tube. The delivery tube 100 may include any cathode light emitter that is not electrically heated by a filament. For instance, a cold cathode fluorescent lamp may utilize a discharge in mercury vapor to emit ultra violet light.

However, in most embodiments, the gases utilized in the tube should be inert for safety. For instance, neon gas vapor may be energized with a 12-volt power supply 120 to generate sufficient UV light. In other examples, other power supplies with various voltages and/or currents will be utilized to develop sufficiently intense light at the current wavelength.

In some embodiments, the light sources 150 may emit x-rays. For these embodiments, the system may include vacuum tubes or x-ray tubes.

The power supply 120 may include an on/off switch or other controls to turn on and off the light sources 150. In some examples, the power supply will include the ability to turn on the UV light source at various intensities, or to modulate the intensity over time depending on the therapeutic application. The power supply may be different for different types of UV light sources 150. For instance, the power requirements for an LED implementation may be less than for a cold cathode implementation.

### UV Ranges

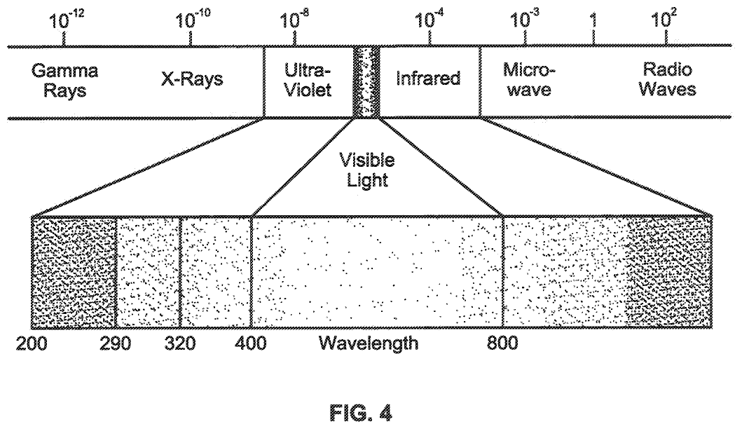

FIG. 4 illustrates UV ranges that may be implemented by the disclosed devices and methods. For instance, the light sources may deliver light only the UVA and UVB ranges, and not in the UV-C ranges. In other examples, the systems and methods may deliver light in all three UV ranges, or also deliver light in the visible spectrum. In some examples, only UVA or only UVB light may be emitted for certain indications and treatments. As indicated above, a light source may have wavelengths of maximum intensity centered around 335 nm, 340 nm, or 345 nm or nearby ranges. In other embodiments, the light sources 150 may deliver light with wavelengths between 320 nm-410 nm, 250 nm-400 nm or other suitable ranges as discussed herein.

In some examples, the wavelength range applied may be the longest wavelength range that is therapeutically effective for a particular application (given the intensity and duration of treatment application). For instance, the shorter the wavelengths, the more likely treatment will damage body cells or tissues of the patient. Accordingly, the longest wavelength that is effective will be the safest to apply.

In some examples, a light source centered around 345 nm or 340 nm (or surrounding wavelengths), may be optimal, as lower/shorter wavelengths are more harmful as they approach the UV-C range. For instance, the shorter the wavelengths, the more energy they have and more likely they are to damage the tissues and DNA of a patient. In some examples, the longest wavelengths that still provide sufficient antimicrobial impacts, making it the safest wavelength that is still effective, may include one or more of the following: 335, 336, 337, 338, 339, 340, 341, 342, 342, 344, 345, 346, 347, 348, 349, or 350 nm. Accordingly, a light source 150 as disclosed herein may emit light with one or more of the preceding wavelengths at intensities that are therapeutically significant. In some examples, a light source may emit UVA with a peak wavelength in a range from 343 nm and 345 nm, which may be utilized for light therapy in patients intubated with a ETT coupled to a ventilator. An example light catheter may include a set of light sources that emit UVA light having a peak wavelength in a range from 343 nm and 345 nm. Further, the light therapy may be delivered at an intensity between 1000 microWatt/cm² and 5000 microWatt/cm² via the light catheter positioned within the ETT tube coupled to the ventilator.

In some examples, the light source may be an LED with a peak wavelength of 335 nm, 336 nm, 337 nm, 338 nm, 339 nm, 340 nm, 341 nm, 342 nm, 343 nm, 344 nm, 345 nm, 346 nm, 347 nm, 348 nm, 349 nm, 350 nm, 351 nm, 352 nm, 353 nm 354 nm, 355 nm. In some examples, the peak wavelength of an LED may have a +/−3 nm, 2 nm, or 1 nm error. In some examples, the LEDs may emit light with significant intensity in a range of +/−2, 3, 4, 5, or 6 nm around its peak intensity emission wavelength. Accordingly, in some examples, the wavelength range of the LED or other light source may be from 340-350 nm (for instance, the wavelength range that includes wavelengths with significant intensity of emission).

In some examples, the light source may be a plurality of LEDs, wherein each of the plurality of LEDs emit a peak wavelength of 335 nm, 336 nm, 337 nm, 338 nm, 339 nm, 340 nm, 341 nm, 342 nm, 343 nm, 344 nm, 345 nm, 346 nm, 347 nm, 348 nm, 349 nm, 350 nm, 351 nm, 352 nm, 353 nm 354 nm, 355 nm. In some examples, the LEDs may emit light with significant intensity in a range of +/−2, 3, 4, 5, or 6 nm around its peak intensity emission wavelength.

In some examples, each of the LEDs may emit light with a beam angle between 100 and 150 degrees. In one example, each of the LEDs may emit light with a beam angle between 120 and 135 degrees.

### Treatment Regimens

The procedures herein may be utilized to treat a number of different inflammatory and infectious diseases. Accordingly, different amounts or time period dosages of UV radiation may be administered depending on the following: (1) type of disease, (2) type of light source, (3) light source power, (4) light source UV range, and (5) severity of the infection or inflammation. For instance, in some embodiments, the time of administration will be determined by the capsule digestion rate, and other factors (e.g., light source power, UV range, and the like) can be manipulated to vary the dosage.

In other examples, the light therapy may be delivered by a caregiver for 10 minutes, 15, minutes, 18 minutes, 19 minutes, 20 minutes, 21 minutes, 22 minutes, 23, minutes, 24 minutes, 25 minutes, 26, minutes 27 minutes, 28 minutes, 29 minutes, 30 minutes, 60 minutes, 90 minutes, 120 minutes, or 160 minutes, any range of minutes between 10 and 160 minutes or other suitable times. In addition, methods of the invention can include administering therapy for a threshold duration of at least 10 minutes, 15, minutes, 18 minutes, 19 minutes, 20 minutes, 21 minutes, 22 minutes, 23, minutes, 24 minutes, 25 minutes, 26, minutes 27 minutes, 28 minutes, 29 minutes, 30 minutes, or 60 minutes. The light source intensity may be at least 1,000 microWatt/cm², 1,100 microWatt/cm², 2,000 microWatt/cm², 2,100 microWatt/cm², 2,200 microWatt/cm², 2,300 microWatt/cm², 2,400 microWatt/cm², 2,500 microWatt/cm², 2,600 microWatt/cm², 2,700 microWatt/cm², 2,800 microWatt/cm², 2,900 microWatt/cm², 3,100 microWatt/cm², 3100 microWatt/cm², 3,200 microWatt/cm², 1,000-5,000 microWatt/cm² or other suitable intensities depending on the application and other factors relevant to the treatment effectiveness. The inventors have confirmed that application of UVA light is safe at intensities of up to 5,000 microWatt/cm². In some examples, the light will be delivered continuously and in other examples it will be incorporated into pulse therapy.

The light source 150 may be various distances from the target based on the intensity and target microbe. For instance, in some examples, the light source 150 may be required to be within 0 to 2 cm from E. coli in order to kill the E. coli (but not at 2.8 cm or 3.5 cm) using an intensity of 2000 microwatt/cm². In some examples, the intensity may be between 1000-5000 microwatt/cm² and the distance to a target tissue may be between 0-1 cm, 0-1.5 cm, 0-2 cm, 0-2.5 cm, 0-3.0 cm, 0-3.5 cm, 0-4.0 cm, or other similar and suitable ranges based on the intensity of the light and target pathogen. In other examples, the timing, distance, wavelength, and intensity required may be different for viruses and other targets.

### Examples

The following examples are provided to better illustrate the claimed invention and are not intended to be interpreted as limiting the scope of the invention. To the extent that specific materials or steps are mentioned, it is merely for purposes of illustration and is not intended to limit the invention. One skilled in the art may develop equivalent means or reactants without the exercise of inventive capacity and without departing from the scope of the invention.

### GI Tract

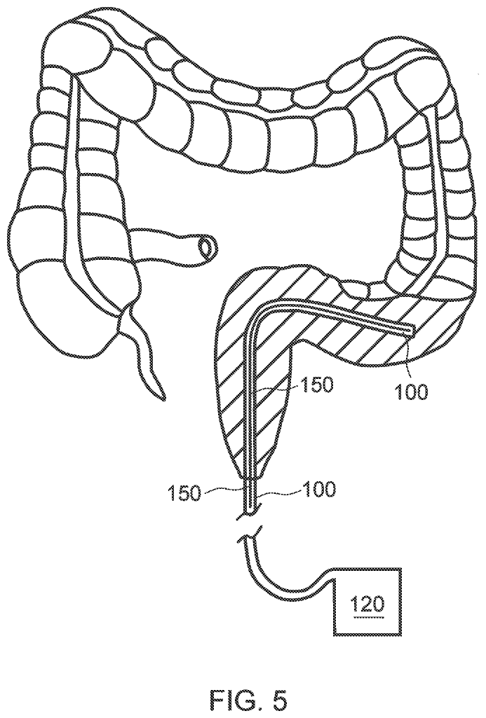

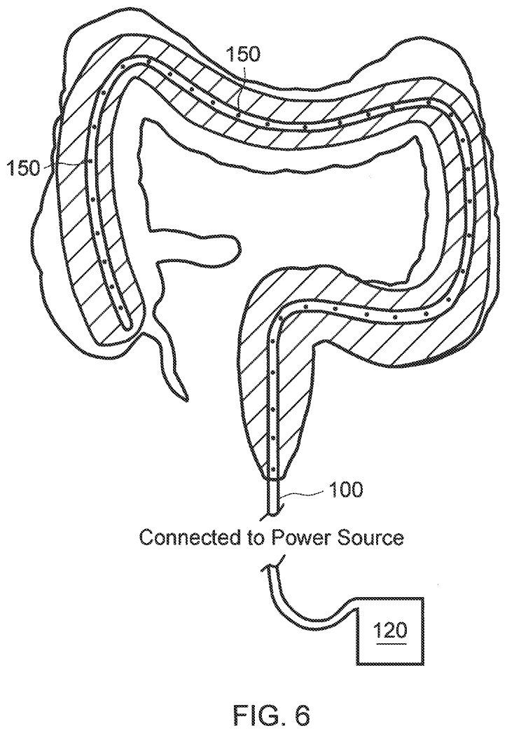

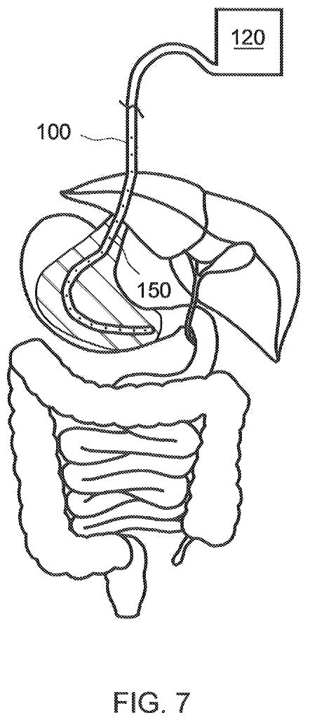

FIGS. 5-6 illustrate example applications to treat disorders in the colon and/or rectum. For instance, FIG. 5 illustrates a delivery tube 100 that includes light sources 150 may be inserted by the caregiver into the colon through the anus. Then, the delivery tube 100 may be navigated to the therapeutic site, for instance the colon, a portion or most of the intestines (see, e.g., FIG. 6), or the stomach via mouth (see, e.g., FIG. 7). Then, the power supply (or light source) 120 may be turned on to illuminate the therapeutic site with UV light.

In some examples, this may be utilized to treat various inflammatory diseases including ulcerative and Crohn's colitis, IBD, infectious diseases and others as more fully described herein. As illustrated, depending on the size, location and type of disease, the delivery tube 100 may include varying amounts of light sources 150 that may be embedded or contained in certain portions or lengths of the delivery tube 100.

FIG. 7 illustrates an embodiment where an endoscope or other delivery tube 100 is inserted through the oral cavity through the esophagus into the stomach. In this example, an infection or inflammatory disease in the stomach may be treated with the UV light sources 150.

### Colonoscopy

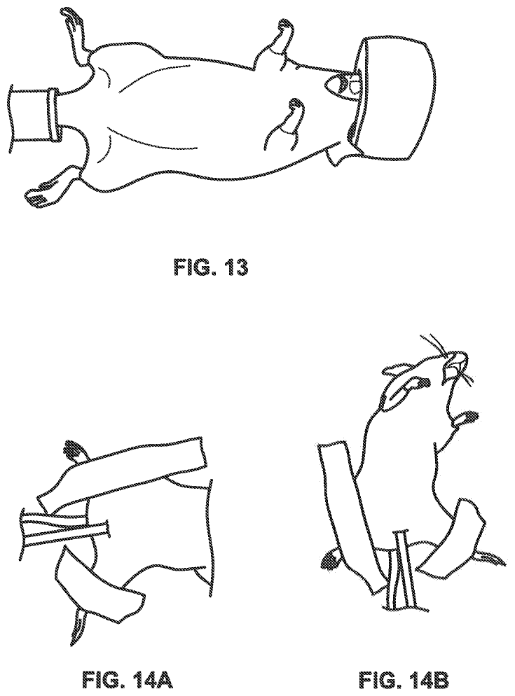

FIG. 13 illustrates an example of a UV emitting device being used on a colonoscopy on a mouse. The colonoscopy and UV application was carried out safely. The parameters have included a normal colonoscopy 72 hours after 10 minutes and 30 minutes of UV exposure with 1,100 micoWatt/cm² intensity.

GI treatments may include the following exemplary applications:

1. Treatment of ulcerative colitis and Crohn's disease and acute/chronic pouchitis and other chronic inflammatory bowel diseases (IBD)

2. Treatment of non-IBD related proctitis

3. Treatment of IBD or non-IBD related fistula

4. Treatment of inflammatory strictures

5. Treatment of microscopic colitis

6. Treating infectious diarrhea using UV light emitting capsules

7. Treating refractory Helicobacter pylori and MALT lymphoma

8. Treatment of esophageal lichen planus and pemphigus vulgaris

9. Treatment of refractory Clostridium Difficile

10. Treatment of colonic inertia, tropical sprue, celiac disease, small intestinal bacterial overgrowth, typhlitis post-bone marrow transplant infections, pseudopolyps (similar to nasal polyps) and radiation enteritis

11. Treatment of Barrett's esophagus with or without dysplasia

12. Treatment of hepatic encephalopathy with daily UV light capsule

13. Treatment of blind loop syndrome in Roux-en-Y patients by placing an ILT (Internal light therapy) catheter through a PEG in the remnant stomach

14. Treating perianal fistulas with transparent setons which can emit UV light

15. Decreasing the rate of infection associated with percutaneous feeding or suction tubes

16. Treatment of gastrointestinal cancers limited to mucosa and submucosa

17. Treatment of hepatobiliary infections, inflammation and cancers limited to mucosa and submucosa

### Capsule

In some embodiments, the delivery device is shaped as a capsule instead of a delivery tube/rod 100. In such embodiments, the capsule is inserted into a patient orally or anally. The capsule can emit light for a certain period. For instance, a capsule can include a smooth clear or semi-transparent polymer or other biocompatible coating to allow for passage of the capsule. In some examples, the capsule may include a light source 150 and a power supply 120. The power supply 120 can include, for example, a small battery. In some embodiments, the capsule can be deployed and pinned to an internal organ to provide prolonged light exposure.

In some embodiments, the capsule is configured such that the UV lights 150 are positioned to emit light in all directions from the capsule. Accordingly, as the capsule traverses the digestive system it will emit UV light in all directions until the capsule is excreted.

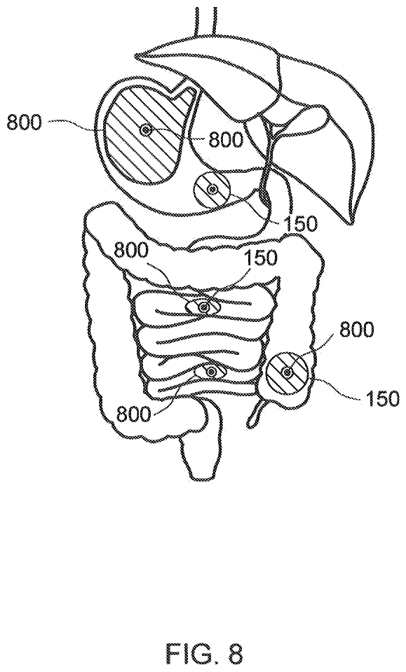

FIG. 8 illustrates an example of a system that utilized a capsule 800 for a delivery device that may be swallowed by the patient. The capsule 800 may contain a light source 150 and a power supply 120 for powering the light source 150. In some examples the capsule will be made, or portions of it will be made of transparent material to allow the light to radiate through the capsule. A capsule may contain a tracking device to assess the location of the capsule inside the gastrointestinal tract. A capsule delivery system may be clipped in a hollow organ for continuous or intermittent controlled delivery.

In some examples, the capsule may be the size of a pill or smaller, and may be orally ingestible. The capsule may include a timer for turning on and off the UV light source when the capsule reaches or is most likely to reach a certain portion of the digestive tract. For instance, the capsule may contain a simple timer to turn on the capsule after 30 minutes, an hour or two hours. For example, the capsule may not turn on the light source 150 until the capsule has reached the digestive tract to treat IBS or other infectious or inflammatory conditions.

### Light Conductive Delivery Tube

In some examples, a light source 150 may be placed inside the delivery tube 100 (e.g. LEDs) and in other examples, a light source 150 may be placed outside or interfacing with a proximal end of a delivery tube 100. Accordingly, in some examples, the delivery tube 100 may be made from fiber optics or other light conductive material to propagate the light from the light source 150 down the delivery tube 100 so that it may be emitted into the treatment site.

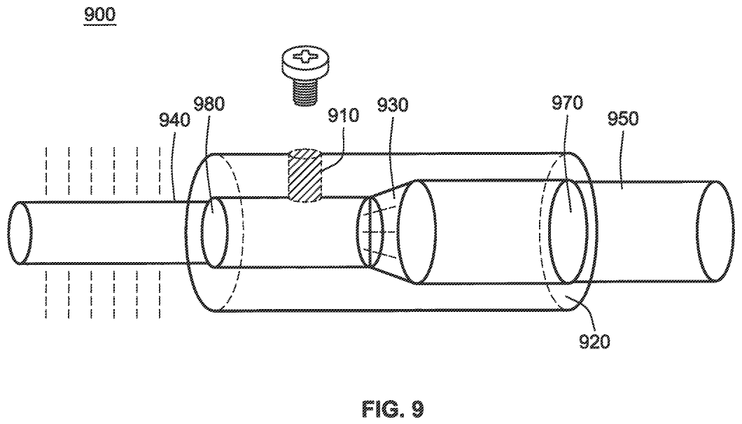

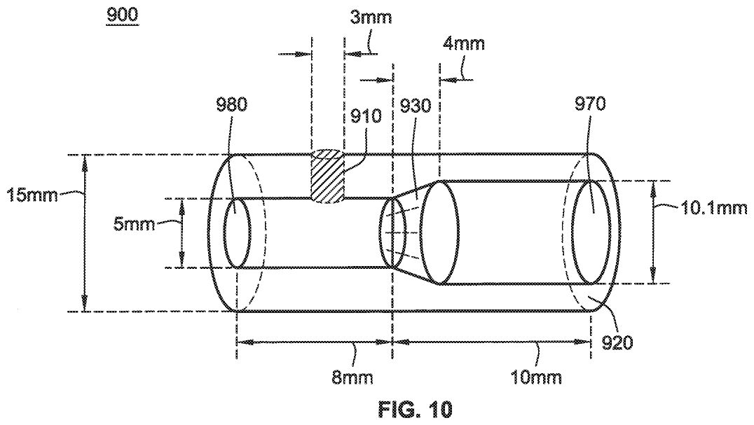

For instance, as shown in FIGS. 9 and 10, a UV light administrative system may include a delivery rod 940, UV light source 950, and a light source attachment 900, wherein the light source attachment 900 is configured to be attached between the UV light source 950 and the delivery rod 940. The delivery rod 940 may include a borosilicate segment 930 which omits UV-C from the light spectrum followed by a segment made out of pure silica (quartz) 900 to extent transmission distance of UV AB with minimal loss.

For example, using only a pure quartz segment has shown to result in significant UV-C light emission (e.g., 4,300 microWatt/cm² UV-C), whereas using pure quartz rod with a short segment of borosilicate in between the UV light source 950 and the delivery rod 940 (e.g., borosilicate filter) results in the same level of detection of UVA and UVB without the borosilicate segment and only 10 microwatt/cm² of UV-C light emitted at the tip of the delivery rod 940, which means that the UV light is reflected back to the body of the delivery rod 940 for a uniform delivery of the UV light throughout the delivery rod 940. The UV light source 950 may be configured to be connected to a power source (not shown) that powers the UV light source 950.

The delivery rod 940 may be a fiber optic rod/catheter. In some example embodiments, the delivery rod 940 is made by scoring using industrial diamond, whereby the glass cutter oil is used and bilateral pressure to snap clearly (rather than opaque) is applied. The tip of the delivery rod 940 may be rounded by a drill (e.g., 500 RPM drill) wherein the drill uses a premium diamond polish pad (e.g., 120-200 grit premium diamond polish pad) and sandpaper (e.g., 400 sandpaper). Afterwards, a body of the delivery rod 940 may be sanded with a 120-200 grit premium diamond polish pad so that the UV-C free light (e.g., UVA and UVB) can emit throughout the body of the delivery rod 940. Alternative chemical opacification can be used for custom opacification of the rod.

The light source attachment 900 may include a body 920 and a fastening mechanism 910 (e.g., a screw, a stopper screw, a fastener, a nail, and the like) that attaches the body 920 to an enclosure (e.g., a rod, a catheter, a handle, or the like). The body 920 may include a front-end aperture 970 that is configured to connect to a light source (or power supply) and a back-end aperture 980 that is configured to connect to a rod (or catheter).

The light source attachment 900 may be made of aluminum for heat conduction and for decreasing light intensity deterioration. The diameter of both the front-end aperture 970 and the back-end aperture 980 may vary in order to fit, e.g., a particular catheter, tube, rod, or the like. The light source attachment 900 may also include a convex lens 930 between the front-end aperture 970 and the back-end aperture 980 that is configured to decrease the light loss. The convex lens may include semi-convex heat resistant lens that decreases light loss and focuses the light.

### Catheter

In some examples, the delivery device may be a catheter tube 100 that may be insertable into the arteries, urethra or other parts of a patient's body. For instance, the catheter tube 100 may include a hollow portion that allows for a guide wire to pass through. Accordingly, a caregiver may navigate a guide wire to the treatment site and then pass the catheter over the guide wire to navigate the catheter to or beyond the treatment site.

The catheter tube 100, like the endoscope implementation, may then contain any variety of light sources 150 suitable for administering UV treatment to the inside of an artery. In some examples, this implementation may use smaller light sources 150 such as LEDs.

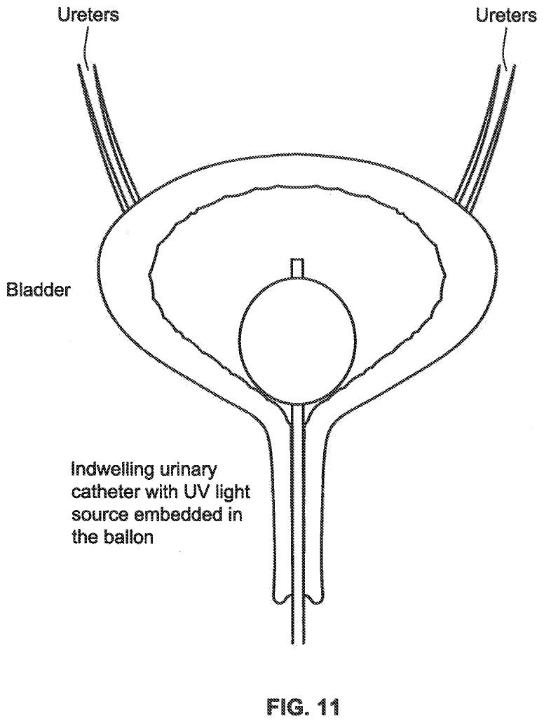

In another example of the present disclosure, the delivery device may be a catheter tube 100 that may be inserted into a bladder as an indwelling urinary catheter (as shown in, e.g., FIG. 11), so that it disinfects the urinary tract infection with UV lights. In another example, the delivery device may be a part of a balloon inserted into a rectum to treat the rectum with UV lights.

### Vaginal

In yet another example, the delivery device may be incorporated into a vaginal rod to treat infection in a patient's vagina.

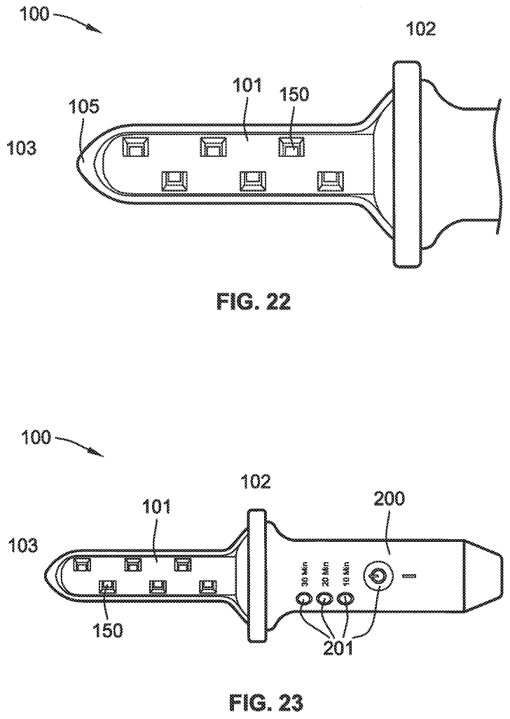

FIG. 22 illustrates an exemplary UV emitting device, in accordance with an embodiment of the present disclosure, that in some examples may be utilized for vaginal delivery of UV light. The UV emitting device can include a delivery tube/rod 100. In some examples, the delivery tube/rod 100 includes a four-sided elongated body 101. The four-sided elongated body 101 can include UV light sources 150 on each of the four sides. The UV light sources 150 can be staggered on each side of the delivery tube/rod 100. The delivery tube/rod 100 can include a proximal end 102 and a distal end 103. The four sides of the elongated body 101 converge into a rounded surface 105 towards the distal end 103. The distal end 103 of the delivery tube/rod 100 is configured for insertion into a patient, as discussed above. In contrast, the opposing proximal end 102 is configured for maneuverability of the delivery tube/rod 100.

FIG. 23 illustrates an example of the UV emitting device of FIG. 22 with a gripping element 200. The gripping element 200 can be configured as a handle. The gripping element 200 can be attached to the delivery tube/rod 100 at the proximal end 102. The gripping element 200 can be designed to be ergonomically sufficient for a physician or a medical provider. The gripping element 200 can also include input components 201 configured to receive a user's inputs. The input components 201 can be connected to an internal processor that alters the functionality of the delivery tube/rod 100 and the UV light sources 150. In some embodiments, the delivery tube/rod 100 includes between 2 and 20 UV light sources. The delivery tube/rod 100 illustrated herein includes three UV light sources 150 on each side of the four sides, for a total of twelve (12) UV light sources 150. It should be understood that other configurations are feasible incorporating the features disclosed herein.

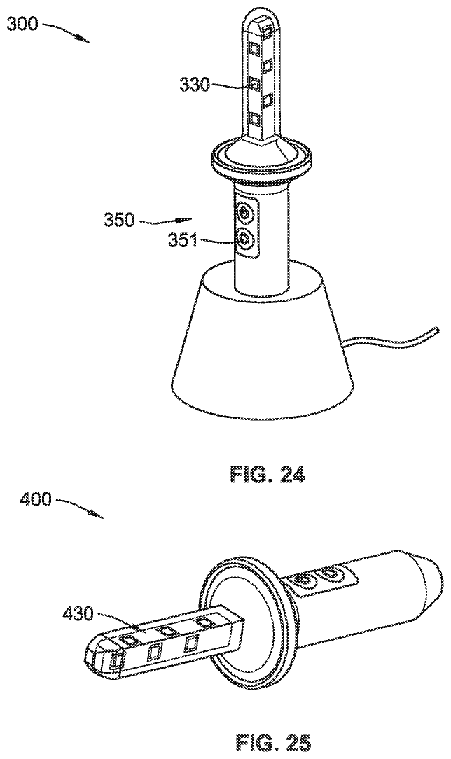

FIG. 24 illustrates an exemplary UV emitting device 300, in accordance with an embodiment of the present disclosure. The UV emitting device 300 can include a gripping element 350. The gripping element 350 can be designed to be ergonomically sufficient for a physician or a medical provider. The gripping element 350 can also include input components 351 configured to receive a user's inputs. The input components 351 can be connected to an internal processor that alters the functionality of the delivery tube/rod 300 and the UV light sources 330. The delivery tube/rod 300 illustrated herein includes two UV light sources 330 on each side of the four sides, for a total of eight (8) UV light sources 330. It should be understood that other configurations are feasible incorporating the features disclosed herein.

In some embodiments, the delivery tube/rod 100 can include a rotating base at its distal end 103. The rotating base can enable rotation of the delivery tube/rod 100 such that light emitted from the UV light sources 150 is uniform. When treating a patient with a rotating delivery tube/rod 100 the uniform UV emittance is likely to assist in treating microbial growth. In some examples, the delivery tube/rod 100 also includes a stepper motor. The stepper motor is able to enable the rotation of the rotating base.

In some embodiments, the UV light sources 150 are distributed along the entire length of the delivery tube/rod 100, and at the distal end 103 to achieve a broader application of the UV light source 150.

In some embodiments, the delivery tube/rod 100 is configured such that the entire delivery tube/rod 100 glows and transmits UV light homogenously. In some embodiments, the delivery tube/rod 100 is configured to emit light waves in the UVA and/or UVB ranges only, and not in the UV-C range. For example, a peak wavelength of the UV light sources 150 can include 340 nm. In other broader embodiments, the delivery tube/rod 100 (and the light sources 150) can deliver wavelengths between 320 nm-410 nm. It should be understood that various wavelengths and various combination of wavelengths can be provided using the disclosed delivery tube/rod 100. Other range wavelengths can include, for example, 250 nm-400 nm. In some embodiments, the vertical illuminated length extends between 8-10 cm around the delivery tube/rod 100.

The delivery tube/rod 100 may be made of any suitable construction (e.g., rigid or flexible), including various polymers that are biocompatible or have a biocompatible coating. FIG. 25 illustrates an exemplary UV emitting device 400, in accordance with an embodiment of the present disclosure. In some embodiments, the delivery tube/rod 100 can include an outer layer of transparent material to allow the UV light from the light sources 430 to radiate outward from the delivery tube/rod 100. In some embodiments, the delivery tube/rod 100 may include an outer surface made from, e.g., silicon, silica, polyurethane, polyethylene, Teflon/PTFE, borosilicate, or other suitable materials. In some embodiments, the delivery tube/rod 100 is constructed using copper with a borosilicate outer layer. For optimal cooling, area of exposure, and uniformity, the delivery tube/rod 100 can include multiple light emitting diodes (LEDs) staggered on a copper bar. In some examples, eight (8) LEDs can be provided on the delivery tube/rod. The spacing of the light sources 430 enables an optimal vertical illuminated length. In some embodiments, the vertical illuminated length extends between 8-10 cm around the delivery tube/rod 100.

By manufacturing the body of the delivery tube/rod 100 using copper, the delivery tube/rod 100 is able to withstand reaching elevated levels in temperature. The copper serves as a heat sink, preventing the delivery tube/rod 100 from reaching uncomfortable temperatures. Applicant also proposes operating the light sources 150 at specific currents to optimize the temperature of the delivery tube/rod 100. In some examples, the light sources 150 are operated within the range of 60-100 mA. Within the proposed range, the temperature of the delivery tube/rod 100 doesn't raise above 40° C., therefore achieving the goal of implementing a proper cooling solution.

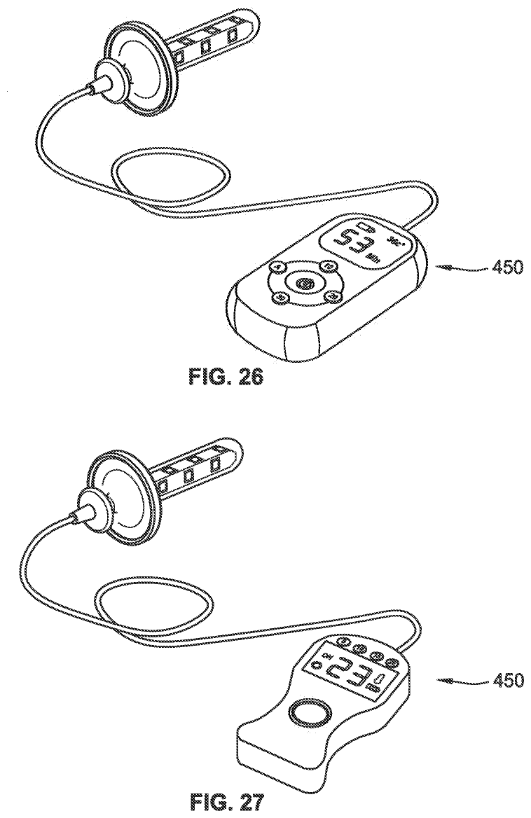

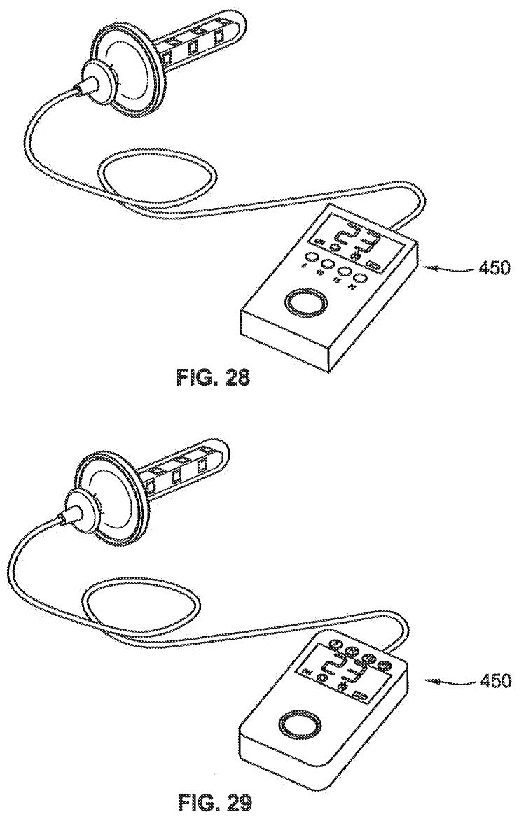

FIGS. 26-29 illustrate various examples of a UV light delivery system with a controller 450. The controller 450 may include one or more processors, memory, and a battery or other power source. The memory may contain instructions with various therapy regimens that may be applied using various intensities and/or durations as disclosed herein. For instance, the memory may contain data structures that when executed by a processor, provide power to the light sources 150 with a given intensity or timing. The controller may be utilized for any of the embodiments disclosed herein, including the vaginal, GI, and ETT based UV light delivery device.

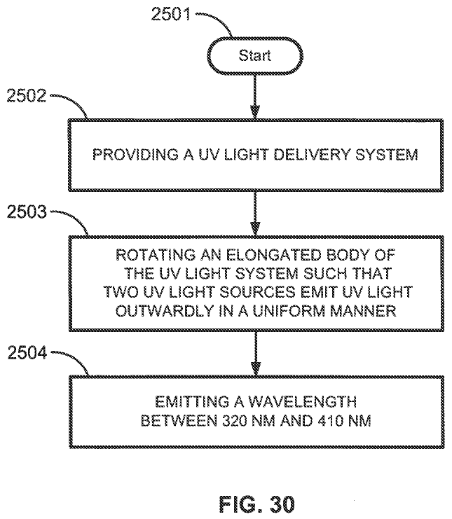

Referring to FIG. 30, a process for performing intra-corporeal ultraviolet therapy is provided. The process includes providing a UV light delivery device, in step 2501. The UV light delivery device includes an elongated body including a proximal end and a distal end. The elongated body includes receiving spaces. The UV light delivery device can also include UV light sources configured to be connected to the receiving spaces. In some examples, the method also includes rotating the elongated body such that the two UV light sources are configured to emit UV light outwardly in a uniform manner, at step 2503.

The process can also include emitting, from the two UV light sources, wavelengths between 320 nm and 410 with a peak wavelength of 340, 341, 342, 343, 344, 345, 346 nm, at step 2504. In some examples, the process also includes emitting, from the two UV light sources, radiation outwardly from the elongated body. In some examples, the elongated body includes four sides. Each of the four sides of the elongated body includes a receiving space, such that corresponding UV light sources 150 are staggered on the elongated body.

The elongated body includes a receiving space and a corresponding UV light source at the proximal end. The elongated body is partially coated with borosilicate glass. In some examples, the elongated body is made up of copper.

### Respiratory

In some examples, the systems and methods disclosed herein may be utilized to delivery UV light to the internal passageways of the respiratory system of a patient. For instance, in some examples, a delivery tube 150 may be navigated into an endotracheal tube (ETT) while a patient is being ventilated. Alternatively, a delivery tube 150 can be navigated into a nasopharyngeal airway (NPA) of a patient. These applications may be utilized to treat or prevent infections, including viral, bacterial, pneumonia, and other infections.

In some examples, the delivery tube 100 may be inserted into the ETT during suctioning of the ETT. In other examples, the systems and methods here may be utilized for improving the treatment of emphysema by equipping chest tubes with a delivery tube to deliver internal light therapy.

For instance, systems and methods have been developed to provide internal ultraviolet therapy in conjunction with an endotracheal tube (ETT) as disclosed herein. Accordingly, the delivery tube 100 may be navigated inside an ETT during ventilation of a patient. In other examples the delivery tube 100 may be connected to or built into an ETT, or an ETT may have light sources 150 incorporated into the ETT. Accordingly, the light sources 150 may be positioned within the tube 150 and/or ETT so that the UV light sources 150 radiate the respiratory tissue in the tracheal airways surrounding the ETT.

For instance, systems and methods have been developed to provide internal ultraviolet therapy in conjunction with a nasopharyngeal airway (NPA) as disclosed herein. Accordingly, the delivery tube 100 may be navigated inside an NPA of a patient. In other examples the delivery tube 100 may be connected to or built into an NPA, or an NPA may have light sources 150 incorporated into the NPA. Accordingly, the light sources 150 may be positioned within the tube 150 and/or NPA so that the UV light sources 150 radiate the respiratory tissue in the nasopharyngeal airways surrounding the NPA.

In some examples, the UV light sources 150 in the delivery tube 100 may be a string of LEDs. For instance, the delivery tube 100 may be a flexible catheter that connects to an ETT or an NPA, and may have LEDs positioned on or inside the catheter to emit UV light outward from the delivery tube 100 to treat the respiratory canals of the patient and/or treat the inside of the ETT or NPA. The LEDs may be connected with a wired connection to a power supply. In other examples, the light sources 150 may be other suitable light sources 150 other than LEDs.

In this example, the LEDs may have a maximum emission intensity wavelength, of 335, 336, 337, 338, 339 340, 341, 342, 342, 344, 345, 346, 347, 348, 349, 350 nm, or any range of wavelengths between 335 and 350 nm. In other embodiments, the LEDs may deliver wavelengths between 320 nm-410 nm, 250 nm-400 nm or other suitable ranges as discussed herein. In some examples, the LEDs may have a peak wavelength between in a range from 343 nm to 345 nm.

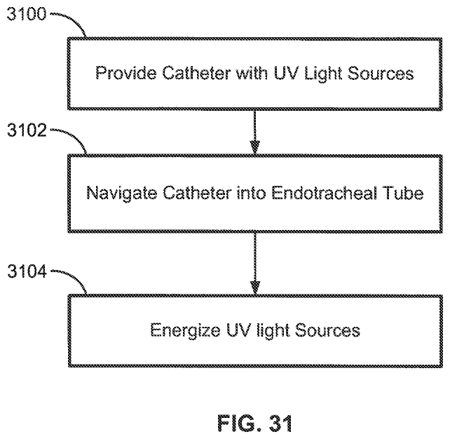

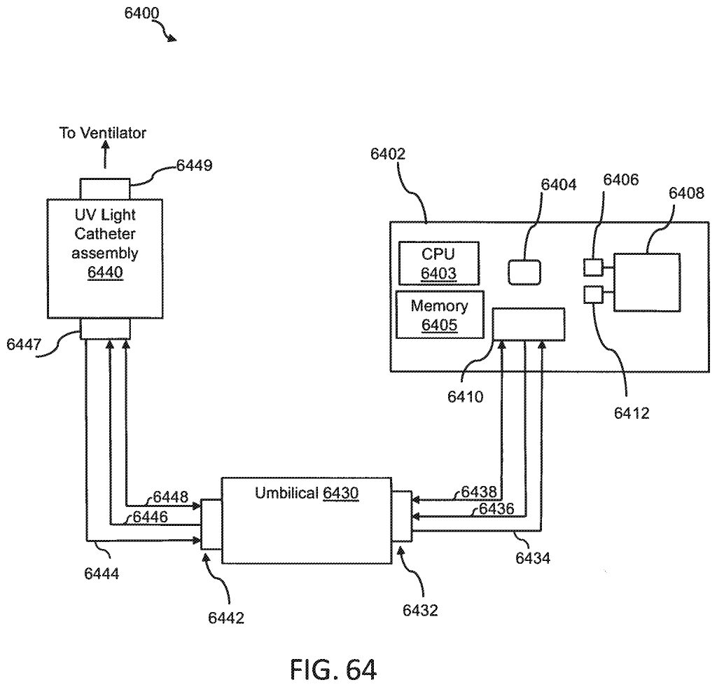

FIG. 31 illustrates a flowchart showing an example of a treatment regimen for treating a respiratory canal and surrounding tissue of a patient with UV light. For instance, a light catheter or other delivery tube 150 with UV light sources may be provided 3100 and navigated into an ETT 3102. In one example, a light catheter assembly including the light catheter or other delivery tube assembly including the delivery tube 150 is coupled to the ETT via an ETT connector portion of the light catheter assembly. An example light catheter assembly is discussed below with respect to FIGS. 64-70. Prior to navigating the light catheter within the ETT, the light catheter is enclosed in a protective sleeve of the light catheter assembly. After connecting the light catheter assembly with the ETT, the light catheter is navigated through a valve (e.g., flap valve) located in the EET connector portion. The light catheter is navigated into the ETT via the valve so that a light emitting portion of the light catheter is within the ETT at a desired depth within the ETT. Said another way, to deploy the light catheter into the ETT, the light catheter is pushed through the valve and into the ETT until the desired depth is reached. Once the desired depth is reached, a secondary seal disposed within the ETT connector portion and surrounding the light catheter may prevent the air from the ventilator from pushing into the protective sleeve. In one example, the secondary seal is in face-sharing contact with a wall of the ETT connector portion and with the light catheter.

Then, the UV light sources may be energized for various treatments 3104. For example, a processor of a control unit communicatively coupled to the LEDs of the light catheter may provide a signal to supply electrical power to the LEDs to energize the LEDs. In some examples, selected LEDs may be energized in order to emit light through a desired length of the light catheter. For example, LEDs may be provided along a first length of the light catheter (e.g., 10 cm), however, LEDs within a second length (e.g., 5 cm) less than the first length may be energize to treat a smaller area. Further in some examples, a first number of LEDs may be energized to output a greater intensity than a remaining number of LEDs, and vice-versa.

In some examples, a delivery catheter with LEDs with wavelengths of maximum emission intensity centered around 339, 340, 341, 342, 343, 344, 345, or 346 nm may be energized for at least 15, 16, 17, 18, 19, 20, 21, 22, 23, 24, 25, 26, 27, 28 29, 30, 40, 60, 80, or 90 minutes (or other suitable time frames in between or outside these ranges) once, twice, or three times daily. The intensity applied may be 1500, 1600, 1700, 1800, 1900, 2000, 2100, 2200, 2300, 2400, 2500, 2600, 2700, 2800, 2900, 3000, 3100, 3200, 3300 uW/cm², or other suitable intensities between or outside these ranges based on the power of the LEDs and the distance to the tracheal or other respiratory canal tissue from the LED light sources.

Further, in some examples, a temperature of the light catheter may be monitored, via a thermistor coupled to the light catheter. In one example, the thermistor may be positioned at least at one LED so as to monitor an LED temperature, which provides an indication of the temperature of the light catheter. As a non-limiting example, the thermistor may be positioned at a last LED, the last LED positioned in a direction opposite to a tip of the light catheter. In some examples, more than one thermistor may be used, each at different locations along the light emitting portion of the light catheter. Responsive to the temperature of the light catheter greater than a threshold temperature, in one example, via the processor, an intensity output of the LEDs may be reduced or power supply to the LEDs may be turned off. In another example, responsive to the temperature of the light catheter greater than the threshold temperature, in addition to or alternative to adjusting LED output, an amount of cool air flow via a cooling tube of the light catheter may be adjusted. For example, responsive to the temperature of the light catheter greater than the threshold temperature, air flow rate through the cooling tube may be increased. While the above examples are illustrated with a single threshold, multiple thresholds may be used for adjusting LED output and/or cooling air flow through the light catheter.

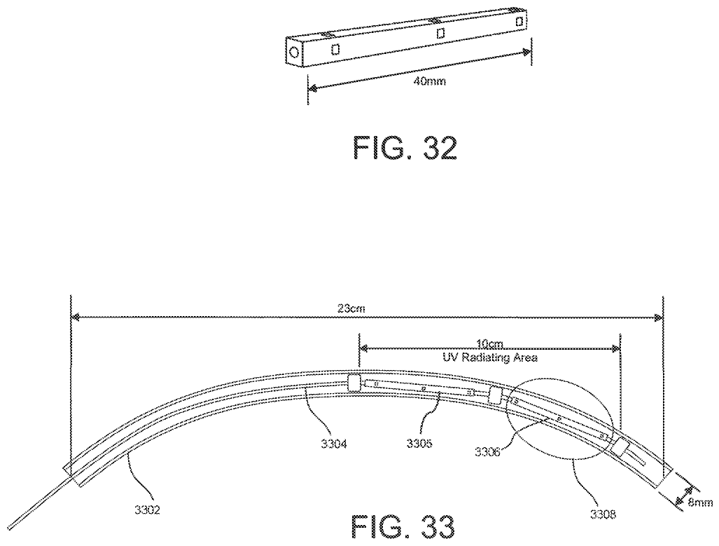

FIGS. 32, 33, 34A-34C, 35A-35C, 36A-36D, and 37 illustrate a few different embodiments that may be employed for a light catheter that may be utilized within the ETT. This may include, as shown in FIGS. 32 and 33 one or more Chip on Board (COB) mini bars that could be connected to or inserted with an ETT 3302. The ETT 3302 may include a balloon 3308 which may reduce an intensity of UV radiation reaching the tissue, and as such, individual COB mini bars may be selectively operated with different intensity in order to account for radiation loss, such as due to ET balloon 3308. For example, a COB mini bar 3306 within the ET balloon 3308 may be operated with a greater intensity than COB mini bar 3305 outside the ET balloon 3308. The whole system could be connected to a flexible metal rod 3304 which may be coupled to a power supply unit (not shown). The present example with one or more COB mini bars shows a UV irradiating area having a length of 10 cm. It will be appreciated that the length may be less than 10 cm or more than 10 cm depending on the application. In some example, one or more additional COB mini bars may be included in addition to mini bars 3305 and 3306 to cover a greater length of the ETT 3302.

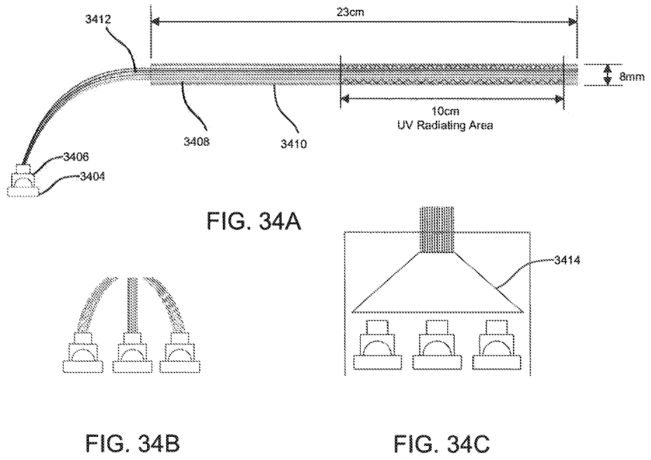

An example fiber optic solution with a COB light engine 3402, that could be integrated with ETT 3410 (or attached to the ETT) to spread out the light therapy is shown at FIGS. 34A-34C. In this example, a single LED (3404 in FIG. 34A) or multiple LEDs (FIGS. 34B and 34C) are connected (e.g., via coupling 3406) to fiber optic cables 3408 which transmit the light to the UV radiating area, and the fiber optics are constructed or treated so that they radiate light in that portion of the tube. Further, in some examples, as shown at FIG. 34C, collimating lens 3414 may be utilized to focus and direct light through the fiber optic cables 3408. As discussed above, the fiber optic cables 3408 may be configured to emit light over a desired length of the ETT 3410.

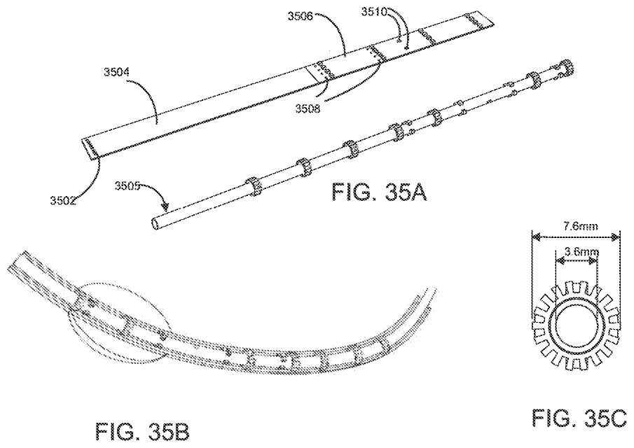

FIGS. 35A-35C illustrate an example of a flexible printed circuit board (PCB) 3504 with a heatsink (FIG. 35C) that includes LEDs 351. In one example, the flexible PCB may be formed into a tube 3505 such that the LEDs 3510 are positioned around a circumference of the tube 3505. This embodiment is helpful for dissipating heat due to large surface area of the flexible PCB. Further, one or more air holes 3508 may be provided to enable improved cooling of the flexible PCB tube. FIG. 35B shows the tube 3505 within an ETT.

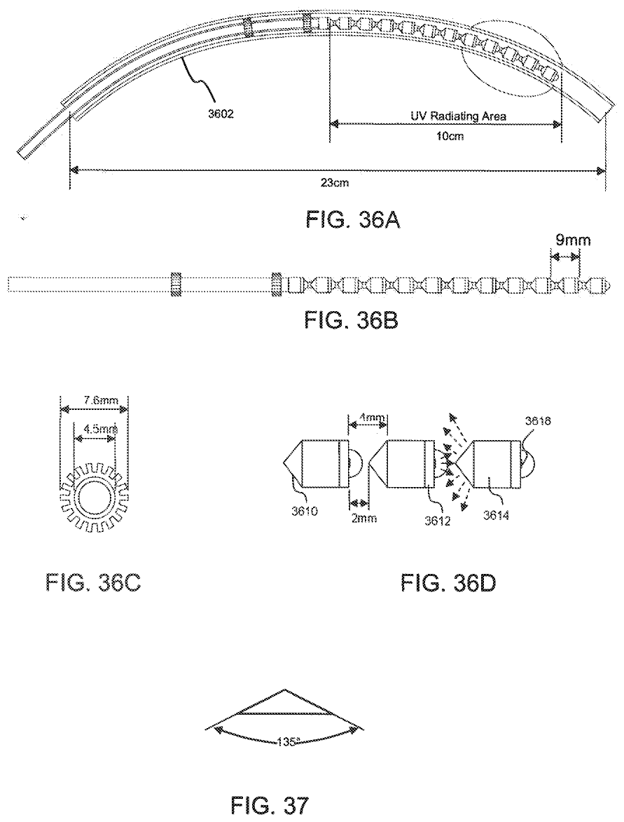

FIGS. 36A-36D illustrates various components of another embodiment of a light catheter including a series of linear reflector and LEDs. Similar to embodiments discussed above, the light catheter which may be navigated through a ETT 3602. As shown in FIG. 36C, the light catheter includes a string of LED units aimed at reflectors nearby. Each LED unit includes an LED 3608, a reflector 3610, and a substrate 3614. An example distance between two LEDs 3608 may be 9 mm and a distance between LED 3608 and an end of the reflector 3610 receiving light from an LED may be 2 mm. For example, the distance between an LED and an end of the reflector may be sufficiently small so as to enable the reflector receive and spread the light out. In this way, greater light distribution is achieved while improving evenness of the light distribution. FIG. 36C shows an example heat sink that may be implemented with the embodiment of FIGS. 36A and 36B. FIG. 37 illustrates an example beam angle of a narrow band (e.g. 343-345 nm) LED that may be utilized in the light catheter shown at FIGS. 36A and 36B.

### UV Light Treatment System

FIG. 64 shows an overview of an example UV light treatment system 6400. In this example, the UV light treatment system 6400 is configured to couple a light catheter assembly 6440 to an endotracheal tube (ETT) of a ventilator and navigate a UV light catheter into the ETT while the ETT is coupled to the light assembly 6440 and the ventilator.

The UV light treatment system 6400 includes a control unit 6402, an umbilical tube assembly 6430, and the UV light catheter assembly 6440. The control unit 6402 includes a compressor 6408 for providing coolant flow through a cooling tube within the UV light catheter assembly 6440 for regulating a temperature of the UV light assembly. The control unit 6402 further includes a valve 6406 and a pressure regulator 6412 for initiating and/or stopping coolant flow, and/or for adjusting a coolant flow rate through the cooling tube.

The control unit 6402 includes a connector 6410 that provides a connection interface for coupling with one or more of a warm coolant connector 6434, a cold coolant connector 6436, and an electrical connector 6438 of the umbilical tube assembly 6430 (at a controller side 6432 of the umbilical assembly 6430).

The umbilical tube assembly 6430 connects the control unit 6402 with the UV light catheter assembly 6440. The umbilical tube assembly 6430 includes an outer sheath within which one or more electrical connection wires for LEDS within the UV light catheter assembly 6440, electrical connection wires to a thermistor of the UV light catheter assembly, and warm and cold coolant tubings are disposed. The electrical connection wires and the cold and warm coolant tubings traverse along a length of the outer sheath. In some examples, the cold coolant tubing may include additional insulation to reduce heat transfer from the environment.

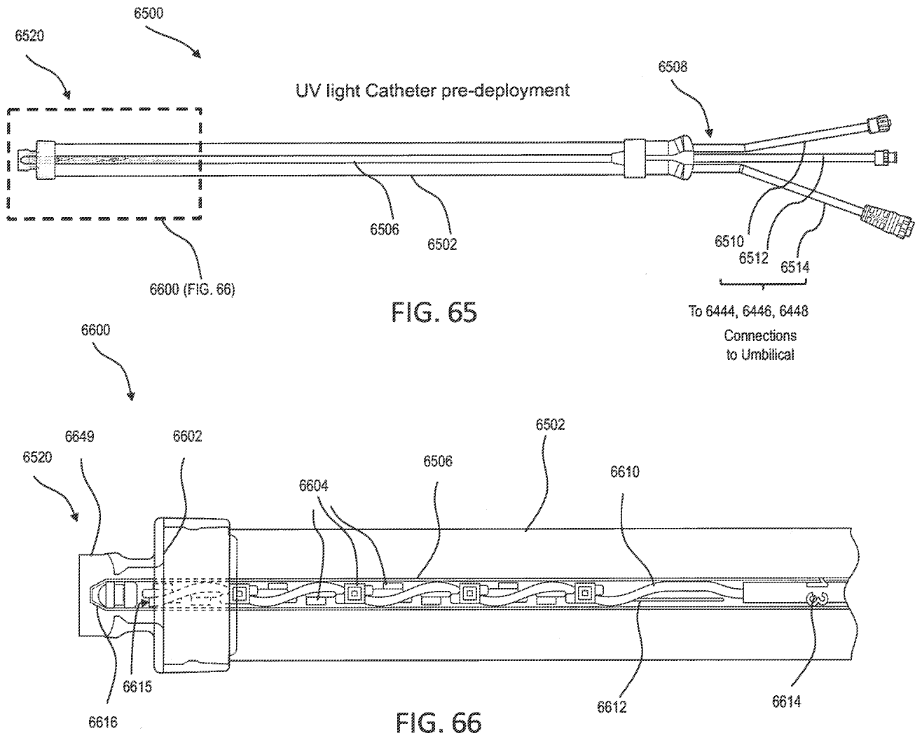

At a UV light catheter side 6442 of the umbilical tube assembly 6430, warm and cold coolant tubings and the one or more electrical connection wires (to the thermistor and the LEDs of the UV catheter assembly) exit as warm coolant connector 6444, cold coolant connector 6446, and electrical connector 6448, which are coupled to corresponding warm coolant, cold coolant, and electrical connectors of the UV light catheter assembly 6440 via a catheter-umbilical connection interface 6447. Details of the UV light catheter assembly 6440 will be described below at FIGS. 65-69.

In one example, an umbilical tube assembly may include one or more air passageways (e.g., warm coolant tubing for returning warm air from a light catheter assembly, a cold coolant tubing for providing cooled coolant to the light catheter assembly) and one or more electrical conductors (e.g., a power supply conductor for providing power supply to the light catheter assembly and/or a thermistor of the light catheter assembly). Further, the one or more electrical conductors may also provide an indication of a temperature of the light catheter assembly from the thermistor to the control unit. In response to the temperature, the control unit may regulate one or more of an operation of the light catheter assembly and coolant flow to the light catheter assembly. The umbilical tube assembly may further include a light catheter connector configured to connect to the light catheter assembly and a control unit connector (or a compressor connector) configured to connect to the control unit (or a compressor system).

The umbilical tube assembly 6430 may be approximately 4, 5, or 6 feet long or other suitable lengths to connect the disposable light catheter to the controller. The umbilical tube assembly 6430 may be long enough to reach from a bedside cart containing the control unit 6402, to a connector on a patient's ETT. As discussed above, the umbilical tube assembly 6430 may include the electrical wires for the LEDs, wires for the thermistor, and tubings for the cooling air to the light catheter assembly 6440 and/or tubings for warm air return from the light catheter assembly 6440. Accordingly, in one example, the umbilical tube assembly 6430 may connect the light catheter assembly 6440 with the control unit 6402 by functioning as a single hybrid connector for transmitting both gaseous coolant and electricity. For instance, a central passageway(s) may transmit air (e.g. cooling air down to the light catheter assembly 6440, and if applicable, return, warm air up to the control unit 6402 along a second passageway). Further, one or more electrical connectors/wires may be spaced around the periphery, or any configuration with respect to the air passageways.

In one example, the coolant is air. Accordingly, cooled air from the compressor 6408 may flow through the cold coolant connector 6436, the cold coolant tubing within the umbilical sheath, the cold coolant connector 6446, and enter the UV light catheter assembly 6440. In one example, air from the compressor may be cooled by a thermoelectric cooler, and flowed in to the cold coolant connector. Further, warmed air from the UV light catheter is then routed back via the warm coolant connector 6444, the warm coolant tubing, and the warm coolant connector 6344, and from there on to the control unit 6402 for recycling, monitoring flow rate, monitoring leaks, and/or expelling to the atmosphere. In some examples, the warm air may be expelled at a connection interface between the umbilical assembly 6430 and the light catheter assembly 6430 or via a valve regulated opening within the umbilical assembly. Details of coolant flow when the UV light catheter is deployed within the ETT is further described below at FIG. 69. In some examples, other gaseous coolants may be used and are within the scope of the disclosure.

The control unit 6402 may include at least one processor (CPU) 6403 and at least one memory 6405 such as read-only memory ROM and/or random-access memory RAM, which comprise computer-readable media that may be operatively coupled to the processor. Thus, the at least one memory 6405 may include system instructions that, when executed by the processor performs one or more of the operations described herein, such as one or more of cooling of the UV light catheter during operation of the UV light catheter within the ETT and controlling operation of UV light according to a temperature of the UV light catheter. Processor 6403 can receive one or more input signals from various sensory components (e.g., a thermistor coupled within the UV light catheter) and can output one or more control signals to the various control components described herein (e.g., to the compressor 6408 within the control unit for regulating flow of coolant through a cooling tube of the UV light catheter, to a power supply coupled to the UV light catheter). The present example shows an example configuration of the control unit 6402, but it will be appreciated that the control unit 6402 may be implemented with other configurations.

In one non-limiting example, the control unit 6402 may contain a medical grade air compressor such the Timeter PCS-414 by Allied, and may output 14 LPM of air at 50 psi or other suitable ranges. As discussed above, the control unit 6402 may include a digital readout, a connector to the umbilical tube (that may be a hybrid connector), and user controls and status indicators. Additionally, the compressor may contain an air valve and pressure regulator. The control unit 6402 may also contain pressure sensors and flow controls for the cooling air, and flow sensors. In some examples the control unit 6402 may provide a closed feedback loop from the thermistors to determine the temperature and/or flow rate of cooling air delivered to the light catheter and through the cooling tube.

FIG. 65 shows an example of a UV light catheter assembly 6500 that may be coupled to an umbilical of a UV light treatment system, such as the umbilical 6430 of the UV light treatment system 6400. The UV light catheter assembly 6500 may be an example of the UV light catheter assembly 6440 shown at FIG. 64. In particular, FIG. 65 shows the UV light catheter assembly 6500 in a pre-deployment configuration. That is, a first configuration prior to coupling to an ETT and being navigated through the ETT.

The UV light catheter assembly 6500 includes a catheter tube 6506 (also referred to herein as light catheter) comprising a light emitting portion 6600 (FIG. 66). When not inserted in the ETT, the catheter tube 6506 is housed in a protective sleeve 6502. A distal end 6508 of the light catheter assembly 6500 is coupled to warm coolant, cold coolant, and electrical connectors of the umbilical via a catheter warm coolant connector 6510, a catheter cold coolant connector 6512, and a catheter electrical connector 6514 respectively. In this way, the umbilical brings in cooling coolant, power supply to a plurality of LEDs and power supply to a thermistor of the UV light catheter assembly (via electrical connector 6514). At a proximal end 6520, the UV light catheter assembly 6500 includes the light emitting portion 6600 (also referred to herein as light delivery portion), which is shown enlarged at FIG. 66 and described below.

Turning to FIG. 66, the proximal end 6520 includes a ETT connector 6649 which houses a proximal tip 6616 of the catheter tube. Further, the ETT connector 6649 directly couples the UV light catheter assembly 6500 to the ETT, which is coupled to the ventilator. The ETT connector 6649 includes a valve that prevents air flow from the ventilator into the light catheter assembly 6500, for example, when the catheter assembly 6500 is coupled to the ETT but the catheter tube 6506 (including the light emitting portion) is not deployed within the ETT. The proximal end 6520 further includes a secondary seal 6602, which prevents air from ventilator from entering into the protective sleeve 6502 when the catheter tube 6506 is deployed within ETT. In one example, the valve is configured as a flap valve. Other types of valves, such as check valves, that prevent back flow of air from the ventilator in to the protective sleeve 6502 may also be used.

The light emitting portion 6600 includes a plurality of LEDs 6604 disposed inside the catheter tube 6506, and a cooling tube 6610 also disposed inside the catheter tube 6506. In one example, as shown, the LEDs 6504 are positioned rotated 90 degrees from each other and facing the catheter tube 6506 such that when the LEDs are electrically powered, the LEDs emit light in a 360 degree pattern outward from the catheter tube 6506 along a length of the catheter tube 6506. Further, in this example, each adjacent LED is rotated 90 degrees. For example, a first LED is at a reference angle of zero degrees, a second LED positioned adjacent to the first LED (that is, the second LED immediately next to the first LED) along a length of the catheter tube 6506 is rotated 90 degrees from the first LED. Further, a third LED adjacent to the second LED along the length of the catheter tube 6506 is rotated 90 degrees with respect to the second LED (which is, 180 degrees with respect to the first LED), and so on such that a Nth LED adjacent to a (N−1) th LED is rotated 90 degrees with respect to the Nth LED, where N is any number depending on a desired length of light emission along the catheter tube 6506. Further, in this example, the LEDs are arranged in a staggered configuration, where each adjacent LED (positioned lengthwise within the catheter tube) is rotated 90 degrees.

In some examples, the LEDs may be arranged in a circumferential configuration. For example, 4 LEDs may be arranged at 90 degrees from each other such that a first LED and a third LED are positioned back-to-back and a second and a fourth LED are back-to-back, the second LED between the first and the third LED, and the fourth LED between the third and the first LED, and the 4 LEDs are not staggered and when electrically powered, the 4 LEDs emit a light at 360 degrees around a circumference of the catheter tube 6506. Another set of 4 LEDs may be positioned at a small distance from the 4 LEDs to provide continuous substantially uniform illumination over a desired length of the catheter tube. In this way a plurality of sets of LEDs may be positioned to cover a desired length of the catheter tube for illumination. Other configurations of the LEDs that cover 360-degree illumination along a desired length of the catheter tube are possible, and are within the scope of the disclosure. For example, when LEDs with a wider beam angle are used, fewer than 4 LEDs may be used to provide 360-degree illumination. As a non-limiting example, 3 LEDs, each rotated 120 degrees from each other, arranged in a staggered manner (that is, adjacently positioned along a length of the tube) or in a circumferential manner (that is, adjacently around a circumference of the cooling tube) may be utilized. In this way, sets of 3 LEDs may provide 360-degree illumination.

Further, the cooling tube 6610 is positioned within the catheter tube 6506 and brings in cooling air to the catheter tube 6506. The cooling tube 6610 has an open end 6615 towards the proximal end 6520 of the catheter tube through which cooling air exits from the cooling tube and circulates back towards the LEDs to cool the LEDs. The cooling tube 6610 is positioned centrally with respect to the LEDs 6604. In particular, the LEDs 6604 are positioned such that a portion of each LED is in contact with the cooling tube 6610. For example, the LEDs 6604 are arranged such that a back portion (e.g., a portion of a LED substrate) is in contact with the cooling tube 6610. In some examples, the LEDs 6604 may be positioned on an inner tube that may include one or more cooling tubes within the inner tube.

Further, the cooling tube 6610 is flexible and winds through the back portions of each LED, which allows for the LEDs to be arranged in a compact manner. As a result, a diameter of the catheter tube is reduced, which is advantageous when deployed within the ETT, as it reduces any resistance to ventilator air flow (to an intubated patient) through the ETT. In some examples, one or more additional openings may be provided for the cooling tube to allow cooling air to exit from one or more additional exit points.

Further, the light catheter tube 6506 may include LEDs that emit peak wavelengths primarily in the 340-350 nm range. An example peak wavelength may be in a range from 343 nm to 345 nm. In some examples, each of the LEDs may emit a peak wavelength of 335 nm, 336 nm, 337 nm, 338 nm, 339 nm, 340 nm, 341 nm, 342 nm, 343 nm, 344 nm, 345 nm, 346 nm, 347 nm, 348 nm, 349 nm, 350 nm, 351 nm, 352 nm, 353 nm 354 nm, 355 nm. In some examples, the LEDs may emit light with significant intensity in a range of +/−1, 2, 3, 4, 5, or 6 nm around its peak intensity emission wavelength.

The catheter tube 6506 has a diameter that is less than an ETT diameter. In one non-limiting example, the catheter tube is approximately 5.4 mm in diameter, which is at or below the diameter of adult bronchoscopes, and is small enough to prevent obstruction of air flow through the ETT. In some examples, the catheter tube may be less than 5.4 mm. Further, the catheter tube 6506 is flexible and can follow the bend of the endotracheal tube when navigated through the ETT. In some examples, the catheter tube 6506 may be sized so that it is at or below the diameter of bronchoscopes. For instance, in adults, the light catheter may be approximately 3, 4, 5, 5.4, 5.5, 5.6, mm or other suitable diameters. Additionally, the catheter's diameter may be sized to prevent obstruction or disruption of the airflow inside the ETT. The helical staggered arrangement of the LEDs with respect to the cooling tube enables the catheter diameter to be sufficiently small so as to provide efficient cooling while reducing resistance to airflow through the ETT.

Furthermore, the cooling tube and LED light arrangement enables a treatment duration to be increased. For example, due to the cooling tube positioned within the light catheter and flow of cooling air within the light catheter, cooling of the LEDs is more effective.

Further, a thermistor 6612 may be coupled to a last LED towards a distal portion of the catheter tube 6506 in order to monitor a temperature of the LEDs 6604. In some examples, one or more thermistors may be used. Further, the thermistor 6612 may be coupled to any of the LEDs 6604. The thermistor 6612 may send an indication of a temperature of the LEDs 6604 to a control unit (e.g., control unit 6402). In one example, a threshold temperature may be used to adjust operation of the LEDs. For example, a single threshold temperature may be used, and when the temperature of at least one LED is at or greater than the single threshold temperature, the LEDs may not be operated. In some examples, when the temperature reaches the single threshold temperature, the control unit may lower the power of the LEDs so as to output a lower intensity of radiation. The control unit may continue monitoring the temperature, and when the temperature decreases below the single threshold, electrical power may be supplied to the LEDs or increased to a desired intensity. In some examples, while the LED is turned off, the coolant flow through the cooling tube 6610 may continue or be increased in order to expedite cooling of the LEDs.

In another example, multiple temperature thresholds may be used for adjusting LED operation and/or coolant flow. As an example, when one or more LEDs within a catheter tube are electrically powered, during a first condition when the temperature of at least one LED is less than a first lower threshold, coolant air flow may not be provided or provided at a low flow rate. During a second condition, when the temperature is at or greater than the first threshold but less than a second higher threshold, coolant air flow may be increased to be greater than the low flow rate. Further, during a third condition, when the temperature is at or above the second higher threshold, electrical power may not be provided to the LEDs (that is, the LEDs may be turned off). Additionally, in some examples, coolant flow may continue when the temperature is at or above the second temperature threshold to enable faster cooling of the LEDs.

Further, in some examples, the catheter tube 6506 of the UV light assembly may be disposable. That is, the catheter may be used for a single patient for the duration of that treatment, and then disposed.

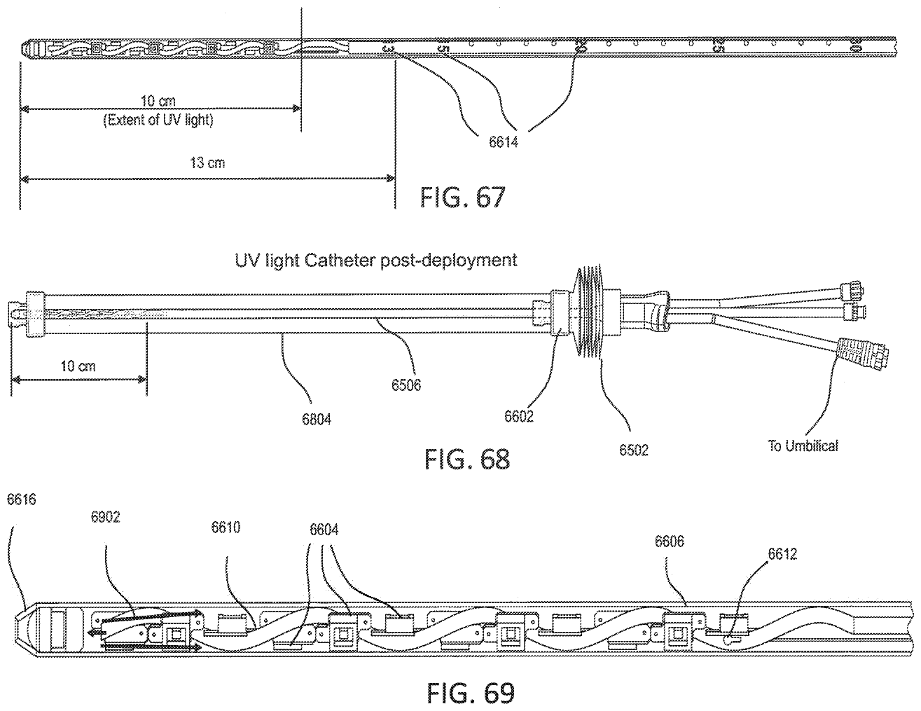

The catheter tube 6506 further includes one or more depth indications 6614 at a non-light emitting portion of the catheter tube 6506. Example indications 6614 are shown at FIG. 67. In particular, the catheter tube 6506 includes one or more external depth markings indicating distance to a proximal end of the catheter. In the example shown at FIG. 67, indications begin at 13 cm and extend to 30 cm. Further, between 15 and 30 cm every 5 cm are marked by a number and the interim distances are marked by circular dots. It will be appreciated that the above example is for illustrative purposes, the different indication types (non-circular shapes, any geometric shape, etc.), and different distances (e.g., distance indications every 2 cm or 3 cm or 4 cm or 6 cm or any usable interval and/or distance marker) may be indicated on the non-light emitting portion of the catheter tube without departing from the scope of the disclosure.

Further, the present example shows the light emitting portion (also referred to as light delivering portion) having a length of 10 cm. It will be appreciated that the length of the light emitting portion comprising the LEDs may be greater or shorter. As a non-limiting example, when the catheter tube is configured for a child, the length of the light emitting portion (that is, the portion that includes the LEDs) may be shorter. Thus, in one example, the length of the light emitting portion may be based on an age and/or height of a patient. Further, depending on an application, such ETT or NPA, etc., the length of the light emitting portion may vary. In some examples, the light delivery portion may be 5, 6, 7, 9, 10, 11, 12, 13, 14, or 15 cm, or other suitable lengths of the catheter tube 6506.

In some examples, the catheter tube may be configured with more than one light emitting portion. For example, one or more light emitting portions, each having a pre-determined length, may be positioned along the length of the catheter tube, and depending on a desired distance of irradiation, the control unit may activate (that is, by electrically powering the LEDs) a desired number of light emitting portions. As an example, if a greater distance of irradiation is desired, a greater number of light emitting portions may be activated by the control unit, and vice-versa. In some examples, the LEDs may be selectively activated. For instance, a number of LEDs activated may be greater when a greater distance of irradiation is desired. Further, LEDs at different locations (e.g., proximal, distal, middle, etc.) may be selectively activated.

FIG. 68 shows the catheter tube 6506 in a deployed configuration within an ETT 6804. To deploy the catheter tube 6506 into the ETT 6804, the catheter tube 6506 is pushed through the flap valve and into the ETT 6804 until the desired depth is reached. Once the catheter tube 6506 is deployed the secondary seal 6602 prevents respirator air from pushing into the protective sleeve 6502. Further, the proximal end 6616 of the catheter tube 6506 may be sealed and have multiple nubs (not shown) which assists in centering the light catheter within the ETT 6804. This provides for more even distribution light distribution inside the trachea.

FIG. 69 shows coolant air flow when deployed within the ETT 6804. The cooling tube 6610 within the catheter tube 6506 brings a steady flow of gaseous coolant towards the end 6616 of the catheter tube, which is sealed. The gaseous coolant (coolant flow indicated by arrows 6902) pushes rearward past the LEDs, keeping the LEDs cool. The warmed air goes out the back of the catheter tube 6506 toward the umbilical assembly. The warmed air can either be expelled at the connection to the umbilical, or brought back to the control unit where the flow rate is monitored, for detecting leaks in the system and/or adjusting flow rate (e.g., increase flow rate for greater cooling).

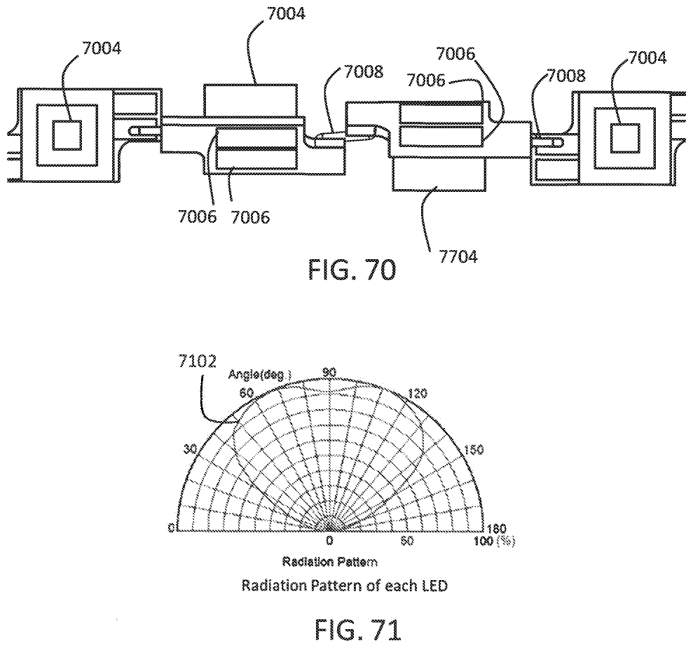

FIG. 70 shows an example LED arrangement that may be implemented within a catheter tube, such as the catheter tube 6506, of a UV light treatment system. Herein, each LED 7004 includes a substrate having copper pads 7006 which function as heat sinks. The present example shows two copper pads 7006, but fewer or more cop Electrical connection between two LEDs is shown at 7008. The copper pads 7006 may be used in addition to or alternative to a cooling tube (e.g., cooling tube 6610 discussed above). FIG. 71 shows an example radiation pattern 7102 of an LED (e.g., LED 7004). For example, the radiation pattern 7102 has a corn-shape. Other radiation patterns may be used and are within the scope of the disclosure.

In some examples, the LEDs are soldered individually onto small PCBs which are connected to each other in series, creating a flexible chain of LEDs. The chain of LEDs can be segmented to create sections that are controllable separately, for example, to emit a higher amount of light in a section under an ETT balloon and hence compensating for the additional light losses or attenuation from the balloon. Further, VIAS on the circuit boards thermally connect the front and back of the PCBs so heat can transfer to the rear of the PCB where there are additional exposed copper pads 7006. These pads are able to expel additional heat to the cooling air in order to keep the LEDs 7004 from over-heating.

The LEDs 7004 may be arranged in a helical pattern within the light catheter. In one embodiment of the helical pattern, each LED is rotated 120 degrees relative to the next LED in the helix and is spaced 3.5 mm apart along the axis of the light catheter in order to produce an even 360 degrees of UVA light around the light catheter.

In one example, the LEDs 7004 are 3.5 mm square and 1.5 mm tall with a flat quartz lens brazed onto the metal housing. Each LED may produce a light pattern of approximately 120 degree as illustrated in FIG. 71. In some examples, the LEDs may have a beam angle between 120 degrees and 135 degrees.

In this way, the UV light treatment system including the UV light assembly, the umbilical, and the control unit discussed above at FIG. 65-70 illustrate an example of the disclosed technology applied to an endotracheal tube, in order to irradiate the respiratory passageways and internal tissues surrounding the passageways of a patient. This may be advantageous to treat coronavirus infections such as infections of SARS-CoV-2 and disease caused by coronavirus infections such as COVID-19 and also decrease the chance of primary infection with SARS-CoV-2 or other viruses in patients who are intubated for reasons other than treatment of COVID-19. Additionally, the treatments may decrease the rate of secondary infections by oral or ventilator associated bacteria or fungus.

As discussed above, the light catheter tube may connect to the umbilical tube, which includes a flexible power and air connector between the light catheter and controller. In some examples, the umbilical tube is reusable. The umbilical thus includes wiring and circuitry to supply power to the LEDs in the light catheter, and a passageway for delivering cooling air from the controller to the light catheter.

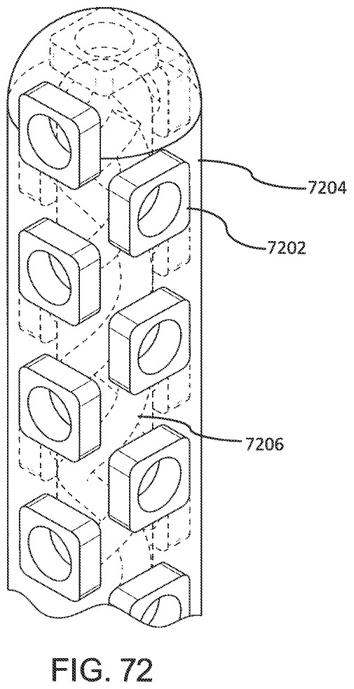

A portion of another example light emitting portion of a light catheter of a UV light treatment assembly that may be used for UVA therapy is shown at FIG. 72. In this example, a plurality of LEDs 7202 is positioned on an inner tube 7204, which may include one or more cooling tubes 7206. An outer tube (not shown) may enclose the plurality of LEDs 7202, the inner tube 7204, and the one or more cooling tubes 7206.

### Nasopharyngeal Airway (NPA)

A device that is inserted into the nasopharyngeal canal is referred to as a “nasopharyngeal airway” (NPA) (alternatively referred to as nasal trumpet or nose hose). The above examples of UV light treatment system, including the control unit, umbilical, and UV light catheter assembly, UVA LED structures, and UVA treatment parameters including wavelengths, intensities, and duration that are described with respect to endotracheal tube may be applied to NPA applications as well without departing from the scope of the disclosure.

In one example, a nose catheter, which may be a catheter or other thin tube or guide wire with UV light sources as disclosed herein may be navigated through the nose to various positions in the respiratory passageways. Accordingly, the therapy applied could, for instance, have antimicrobial effects in the canal, for instance, prior to a patient needing ventilation for a pneumonia-like infection.

### Additional Respiratory Applications

The discloses systems and methods may be utilized for the following additional respiratory applications:

1. Placing an Internal Light Therapy (ILT) tube while using an ETT to eliminate the bacteria in the tube and also the bacteria accumulating around the larynx and other tissues to prevent pneumonia.

2. Build ETT with ILT capability with intermittent emission.

3. Improving the treatment of emphysema by equipping chest tubes with ILT.

### Additional Treatment Applications and Regimens

The procedures herein may be utilized to treat a number of different inflammatory and infectious diseases. Accordingly, different amounts or time period dosages of UV radiation may be administered depending on the following: (1) type of disease, (2) type of light source, (3) light source power, (4) light source UV range, and (5) severity of the infection or inflammation. For instance, in some embodiments, the time of administration will be determined by the capsule digestion rate, and other factors (e.g., light source power, UV range, and the like) can be manipulated to vary the dosage. In other examples, the endoscope may be delivered by the physician/surgeon for an hour, 30 minutes, two hours, or other suitable times.

Following are examples of treatment regimens and their applications. Accordingly, the devices and methods disclosed herein may be adapted to treat these different conditions.

### Urology and Nephrology:

1. Sterilizing blood in patients with known bacteremia, fungemia or viremia during dialysis to eradicated or to decrease the microorganism load. Alternative a light needle can be placed in fistula to be turned on even outside of dialysis window. Ex vivo sensitivity analysis will be done for narrower wavelength but more intense ILT.
2. Sterilizing indwelling urinary catheters in catheter dependent patients
3. Treatment of bladder and urethral cancer limited to mucosa and submucosa
4. Treatment of refractory cystitis/urinary tract infection
5. Adding UV phototherapy to peritoneal dialysis catheter to decrease the risk of peritonitis and even long term peritoneal sclerosis.

### Cardiology

1. Sterilizing blood in patients with known bacteremia, fungemia or viremia with LVAD to eradicated or to decrease the microorganism load. Alternative a light needle can be placed in fistula to be turned on even outside of dialysis window. Ex vivo sensitivity analysis can be done for narrower wavelength but more intense UV therapy.
2. Refractory bacterial and fungal endocarditis being treated with direct UV light exposure of valves. A photosensitizer may be given intravenously in this case.

### Dentistry

1. Treatment of gingivitis.
2. Treatment leukoplakia and oral lichen planus.
3. Treatment of cancers limited to mucosa and submucosa

### Hematology/Oncology

1. Treatment of intestinal Graft-versus-host disease. X-ray wavelength will be emitted in this case, leading to death of lymphocytes. This can be used in patients with end stage Crohn's disease awaiting small bowel transplant or palliative care.

### ENT

1. Treatment of chronic sinusitis.
2. Treatment of chronic otitis.
3. Treatment of acute otitis media in patients requiring tympanostomy.
4. Treatment of nasal polyps.
5. Treatment of halitosis.
6. Treatment of recurrent tonsillitis/pharyngitis.
7. Treatment of cancers limited to mucosa and submucosa.

### Surgery

1. Improving the treatment of abscesses by equipping the drains with UV light technology.
2. use with surgical drains to avoid superimposed infection.
3. Accelerating anastomosis healing process.
4. Aid in preventing adhesions.

### Neurosurgery

1. Intrathecal fibro-optic delivery of UV light in treatment of refractory meningitis.
2. Treatment of refractory shunt infections.
3. Treatment of prion diseases with intrathecal or subarachnoid UV therapy. 4—Treating JC virus related Progressive multifocal leukoencephalopathy by decreasing viral load.

### Gynecology

1. Treatment of bacterial or fungal vaginosis.
2. Treatment of rectovaginal/colovesical fistula.
3. Treatment of cancers limited to mucosa and submucosa

### Rheumatology

1. Intraarticular ILT for treatment of inflammatory and infectious large joint arthritis.

### Vaginal Therapy

1. FIGS. 14A, 14B illustrate an example of a UV emitting device being used on a vaginal treatment of a mouse.

### EXPERIMENTAL DATA

The following set of experimental data is provided to better illustrate the claimed invention and is not intended to be interpreted as limiting the scope.

### Example 1: E. coli

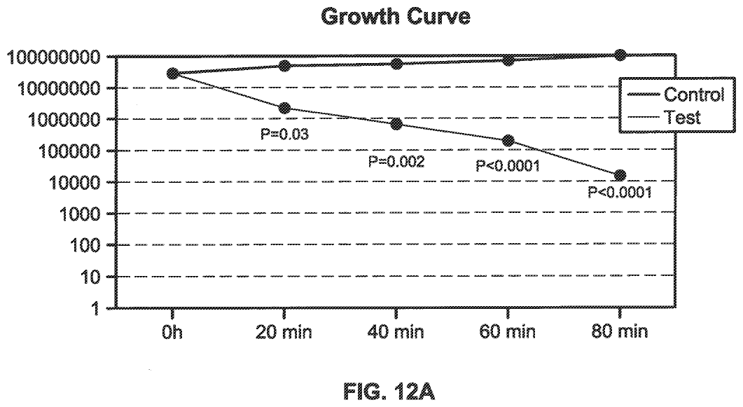

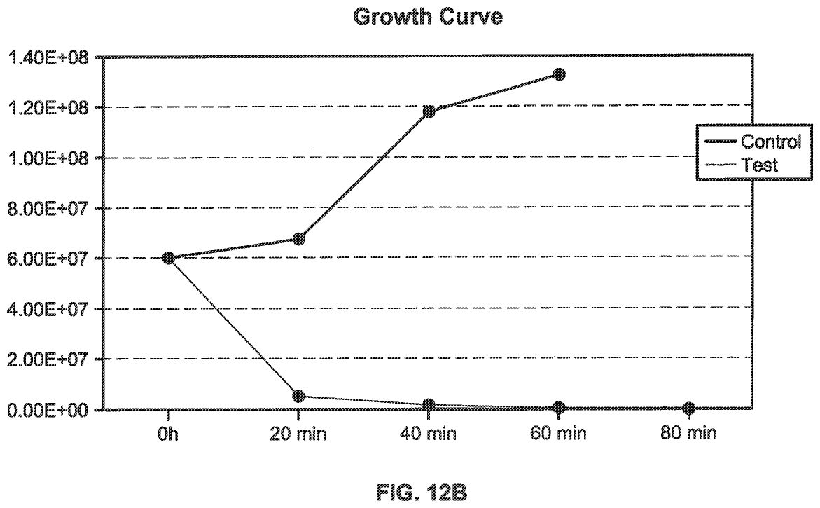

FIGS. 12A and 12B illustrate experimental data showing an example of a UV emitting device of the present disclosure being used to prevent E. coli from proliferating. As shown, the control group where the UV light was not applied continued to grow, whereas the test group that had UV light applied through the UV emitting device showed continuous decrease in E. coli count over time. The UV light is shown to both prevent E. coli from proliferating and also kill the bacteria over time.

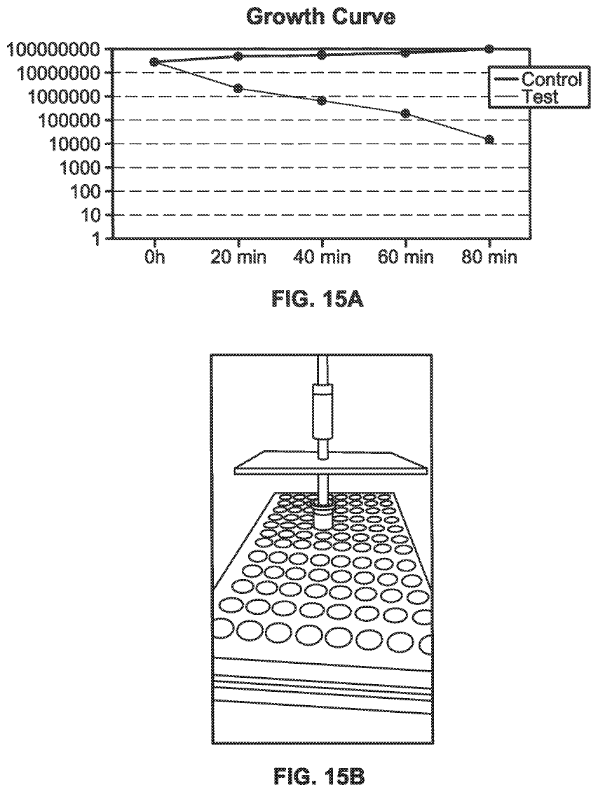

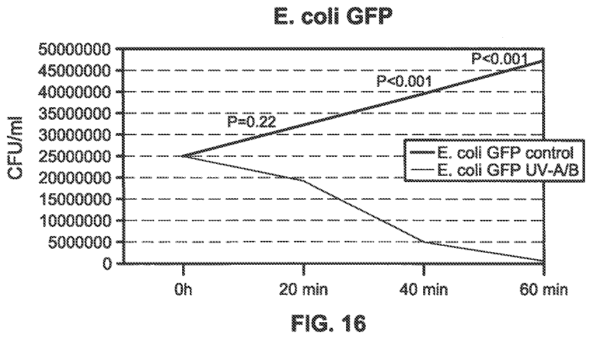

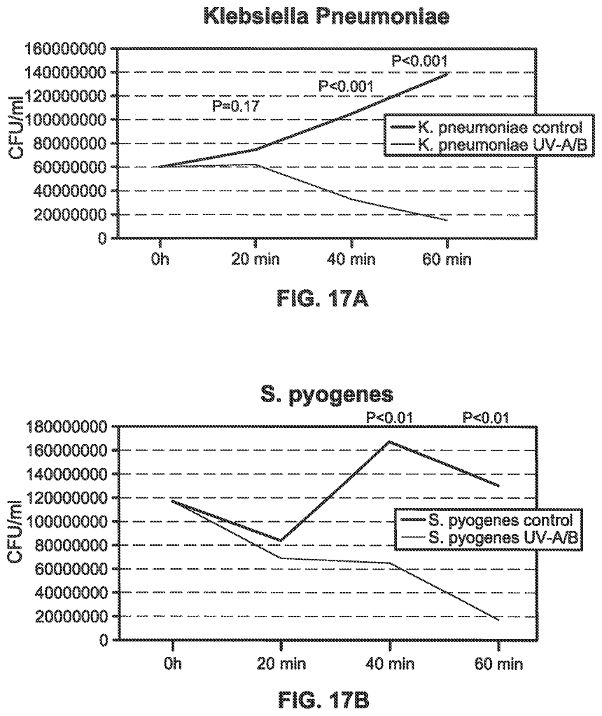

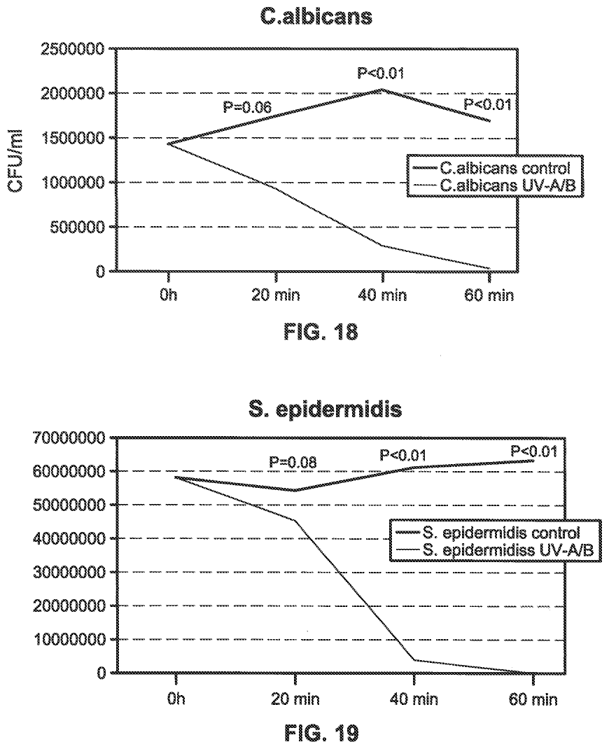

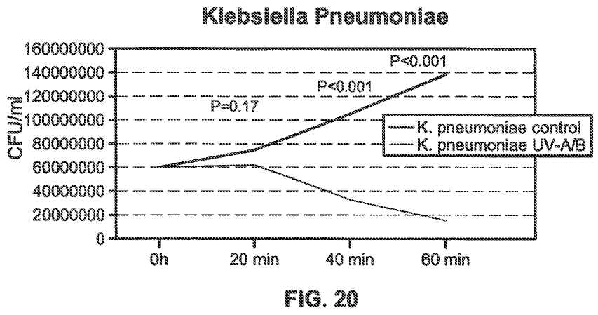

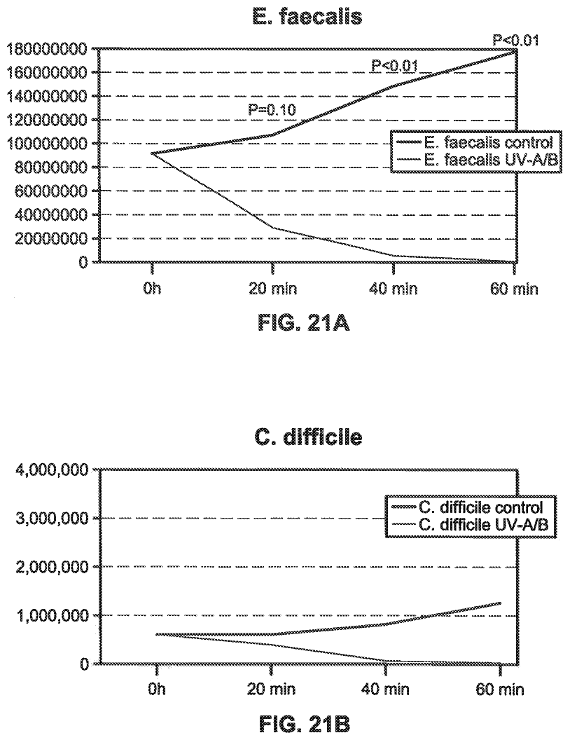

FIG. 15B illustrates an example of a UV emitting device of the present disclosure being used on a liquid culture containing E. coli. The results of this experiment and similar experiments with other bacteria and a fungus, C. albicans are shown in, e.g., FIGS. 15A, and 16, 17A-17B, 18-20, 21A, and 21B. All of the results illustrate a significant reduction in the growth of E. coli and other infectious agents in liquid samples where UVA and UVB lights were emitted by the UV emitting device of the present disclosure onto the liquid samples.

### Example 2: Bacteria

In another example, two exemplary devices according to the present disclosure were used in UVA experiments to treat bacteria. The first device was a borosilicate rod (outer diameter 3 mm) repeatedly etched with a mixture of diluted sulfuric acid, sodium bifluoride, barium sulfate and ammonium bifluoride, with a reflective coating added to the end of the rod through which UVA was side-emitted. This process resulted in a side glowing rod of UVA (peak wavelength of 345 nm) as confirmed by spectrometer (Ocean Optics; Extech). The second device incorporated narrow band LEDs with a peak wavelength of (345 nm).

The UVA rod was inserted into liquid media. A mercury vapor lamp served as light source (Asahi Max 303, Asahi Spectra Co., Tokyo, Japan). The second UVA light-emitting device was a miniature light-emitting diode (LED) array (peak wavelength 345 nm) mounted on a heatsink (Seoul Viosys, Gyeonggi-Do, Korea). This device was used for the plated experiments noted below.

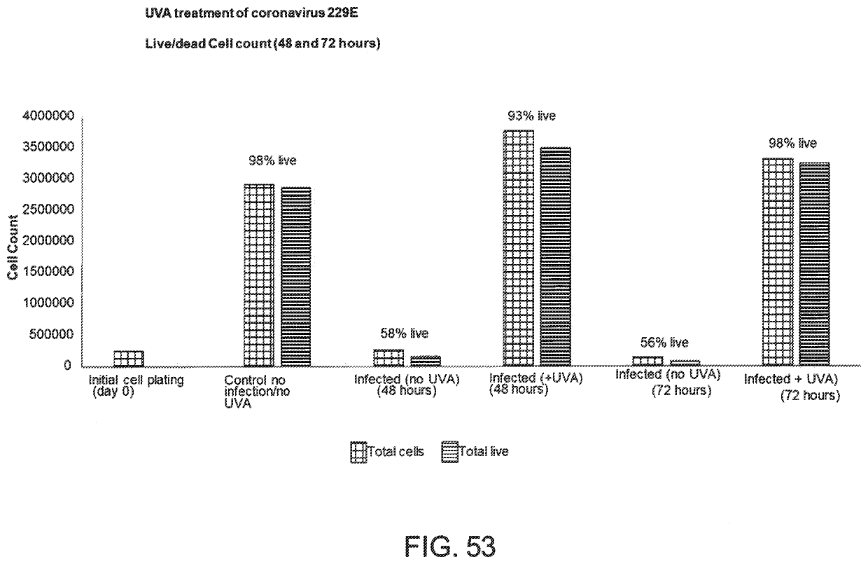

Stock cultures of Escherichia coli, Escherichia coli GFP, Pseudomonas aeruginosa, Streptococcus pyogenes, Staphylococcus epidermis, Klebsiella pneumoniae, Enterococcus faecalis, Proteus mirabilis, Clostridioides difficile and Candida albicans were grown in appropriate liquid culture media and conditions as illustrated in the table shown in FIG. 53. The American Type Culture Collection (ATCC) strains and one clinical isolate were grown in appropriate solid and liquid media following instructions suggested by the ATCC for each microorganism (Manassas, Va., USA). Using sterile techniques, the vial containing the microbial strain was opened and the entire pellet was rehydrated with approximately 500 μL of liquid broth.

Aseptically, the resuspended pellet was transferred to a tube containing 5-6 mL of the same liquid broth used to resuspend the cells. Several drops of the primary broth tube were used to inoculate a solid microbial agar and isolate single colony forming units (CFU). The liquid and solid cultures were incubated at specific temperatures, atmospheric conditions and times described in FIG. 53.

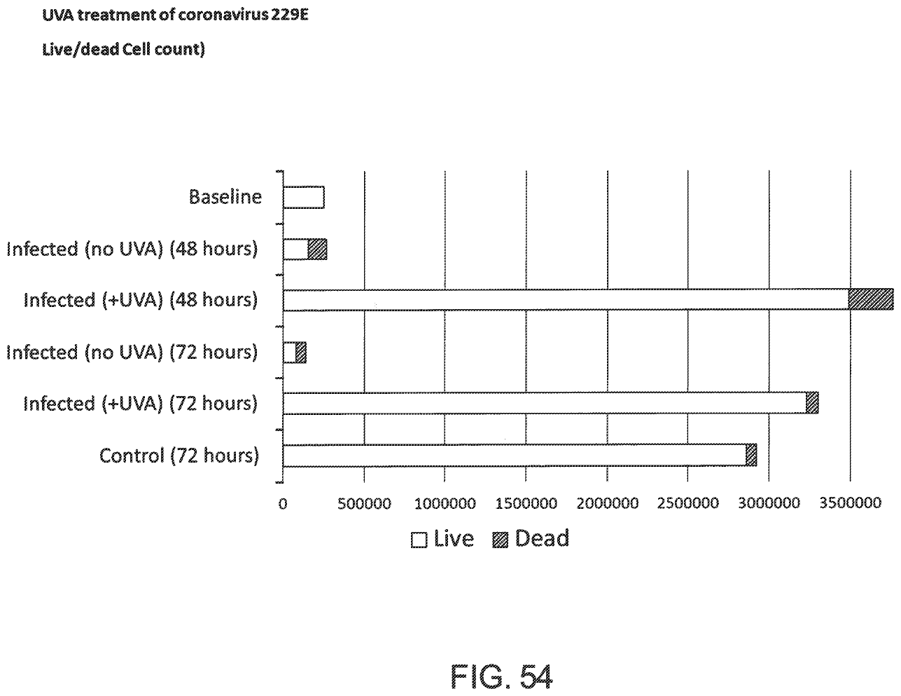

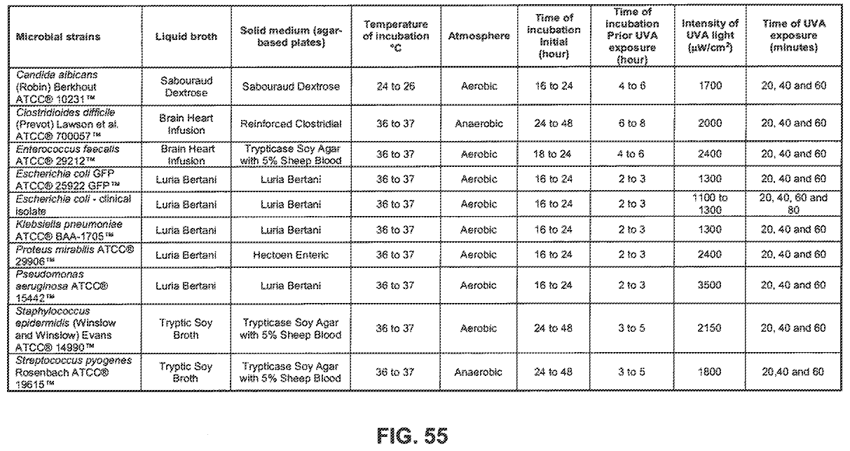

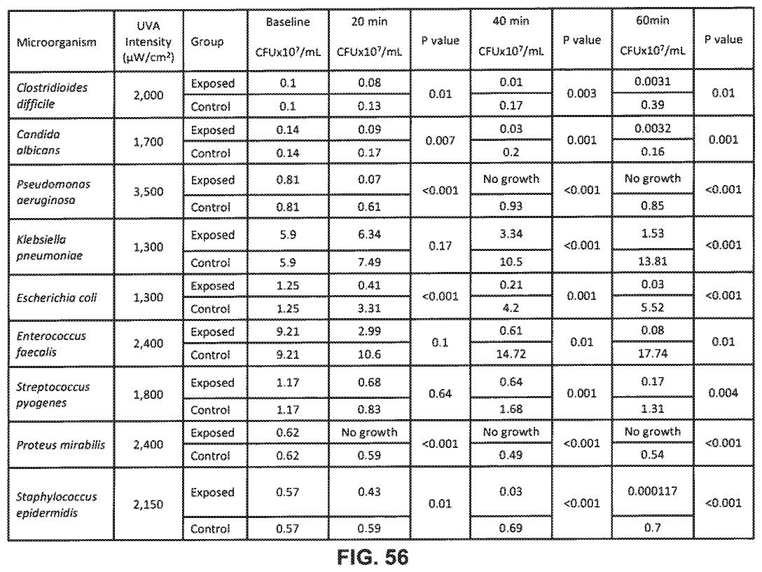

Initially, a liquid culture was prepared from a single CFU of each microbe to guarantee the purity of the strain during the UVA therapy. Only new pure liquid cultures were used during the experiments. One single colony was added to a 10 mL sterile tube containing 5 mL of liquid medium followed by thorough vortexing to homogenize the microbial cells. The liquid cultures shown in FIG. 53 were incubated until they reached the McFarland standard of 0.5. After meeting the standard turbidity, microbial cultures were mixed thoroughly for one minute and 1000 μL of the liquid culture were transferred into two 1.7 mL micro-centrifuge sterile tubes to be used as the treatment and control. An aliquot of 100 μL of each tube was serially diluted and plated on solid microbial medium to determine the number of CFU/mL at baseline as shown in FIG. 54.

Prior to UVA light therapy, several sterile 1.7 mL tube caps were prepared by creating a small hole through the top using a heated glass rod. The hole had the shape and size of the rod used to transmit UVA light.

Aseptically, the original caps from the liquid cultures in 1.7 mL tubes were replaced with the sterile caps with the hole. The UV light transmitter rod (sterilized with 70% ethanol) was placed into the hole created on the top of each cap. An identical rod was also placed into control-tubes. The light was transmitted through the glass rod inserted into the tube using the MAX-303 Xenon Light Source (Asahi Spectra USA, Inc., Torrance, Calif.). UV band width and irradiance peaks were assessed (Flame UV-VIS Fiber Optic Spectrometer, Ocean Optics). UV intensity was measured with SDL470 and UV510 UV light meters (Extech, N.H., USA) Extech). Absence of UVC was confirmed using SDL470 UV light meter (Extech N.H., USA). FIG. 53 describes the intensities and exposure durations of UVA light applied to the bacterial cultures.

After the end of the treatment time, the rods were removed from the treated and control tubes and a new sterile cap without a hole was used to close the liquid cultures. Both the treated and control groups were homogenized by vortexing. An aliquot of 100 μL of each tube was then serially diluted and plated on solid microbial medium to determine the number of CFU/mL after UVA treatment as shown in the table depicted in FIG. 54. This process was repeated until all time-points described in FIG. 54 were accomplished.

After each time point (baseline and post UVA treatment), 100 μL of the liquid microbial cultures (treated and controls) were serially diluted into sterile 1×PBS (EMD Millipore, Billerica, Mass.). The final serial dilution factors were 1:10 (100 μL of microbial culture and 900 μL of sterile 1×PBS), 1:100, 1:1000, 1:10,000 and 1:100,000. 100 μL of each dilution were plated in duplicates onto solid agar plates and incubated at time, temperature and atmospheric conditions described in FIG. 53. After incubation, the colonies were counted using a Scan 300 automatic colony counter (Interscience, Woburn, Mass., USA), and the numbers of CFU/mL were defined after correcting for volume and the dilution factor.

The second device utilized in these experiments incorporated a miniature light-emitting diode (LED) array (peak wavelength 345 nm, bandwidth 10 nm) mounted on an aluminum heatsink (Seoul Viosys, Gyeonggi-Do, Korea). In the first experiment, this system was placed at 1 cm from the surface of a culture plate with a thick lawn of E. coli at approximately 2000 μW/cm² for 20 minutes. Subsequently, this light source was applied to liquid culture of 10² CFU/mL of E. coli and P. Aeruginosa in separate experiments.

For both conditions, UVA was tested in separate sets of experiments at intensities of 500, 1000, 2000 and 3000 μW/cm² for 20 and 40 minutes at 1 cm to produce a dose response curve. After incubation, the colonies were counted and colony sizes were measured using a Scan 300 automatic colony counter (Interscience), and the numbers of CFU/mL were defined after correcting for volume and the dilution factor.

### Results

Exposure to UVA was associated with a significant reduction of various pathogenic microbes, including Candida albicans (P=0.007) and Clostridium difficile (P=0.01) as illustrated in the table depicted in FIG. 54. A UVA light exposure time of 20 min (intensity ranging 1300 to 3500 μW/cm²) was the minimum effective duration to observe reductions for most microorganisms tested when compared to controls (P<0.05), except Klebsiella pneumoniae (P=0.17), Enterococcus faecalis (P=0.1), and Streptococcus pyogenes (P=0.64). The UVA light exposure times of 40 and 60 min were effective against all microorganisms tested when compared to untreated controls (P<0.05, FIG. 33). Notably, the bactericidal and fungicidal effects exhibited a dose-dependent response to UVA light, with greater microbial reductions associated with longer exposure times as illustrated in FIG. 54.

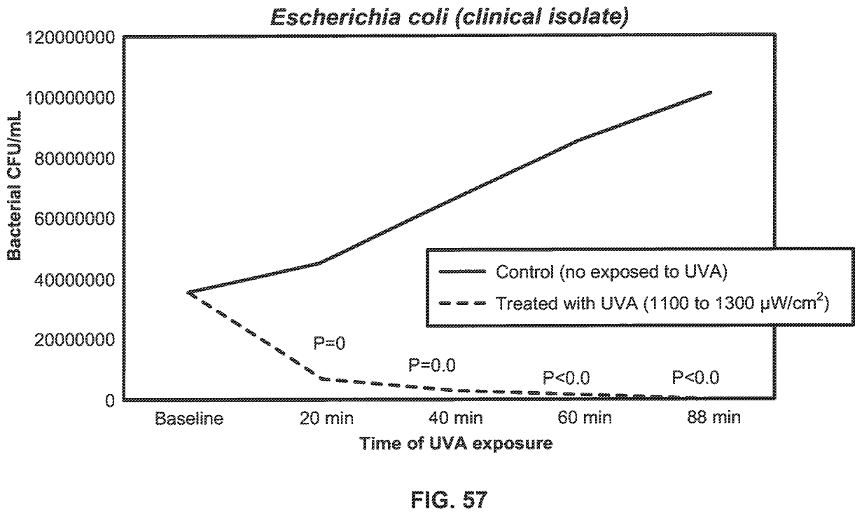

UVA light treatment was also applied to a clinically isolated Escherichia coli strain obtained from a human urinary tract. UVA light was tested in a set of five consecutive experiments, exposing this bacterial culture to 20, 40, 60 and 80 minutes of UVA, 1100 to 1300 μW/cm². Compared to baseline, the number of CFU/mL observed in bacterial cultures exposed to UVA light decreased at all-time points evaluated, including 20 min (P=0.03), 40 min (P=0.0002), 60 min (P<0.0001) and 80 min (P<0.0001) as shown in FIG. 57.

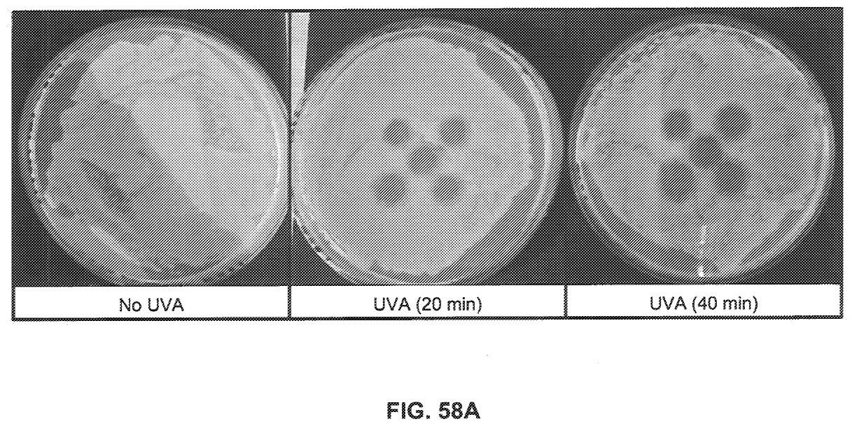

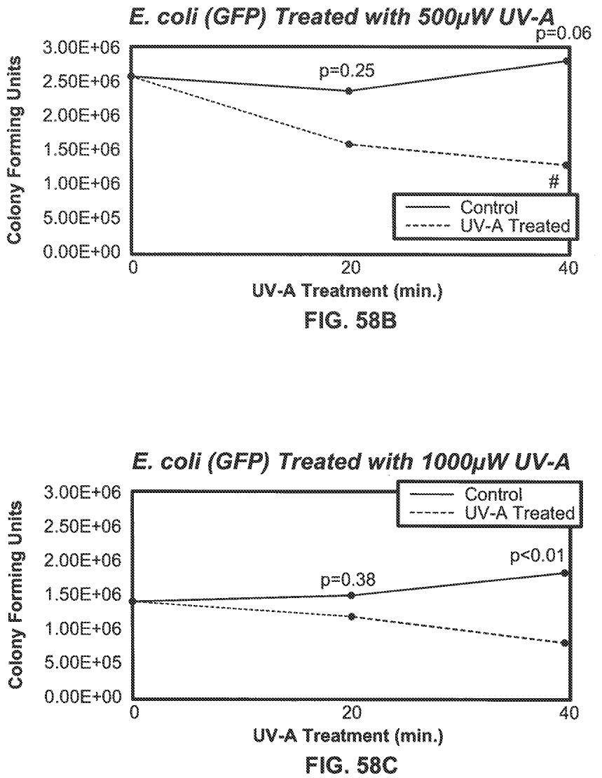

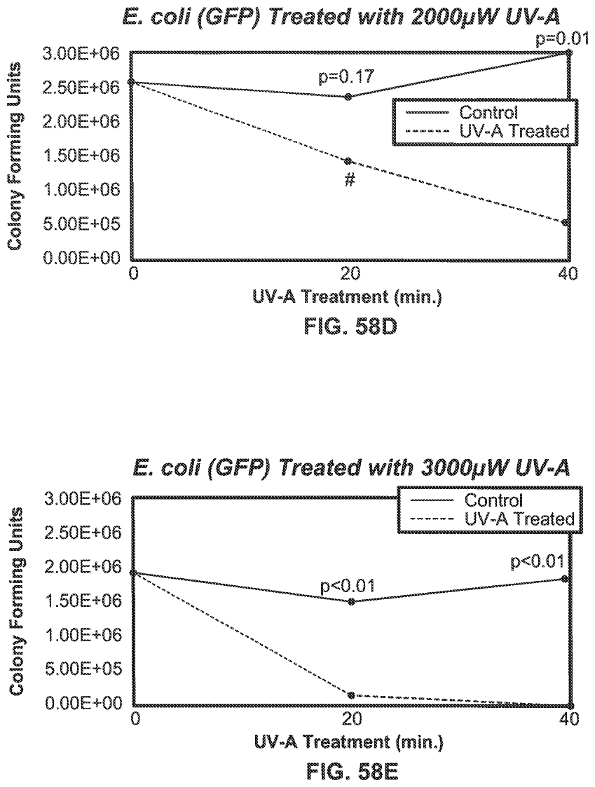

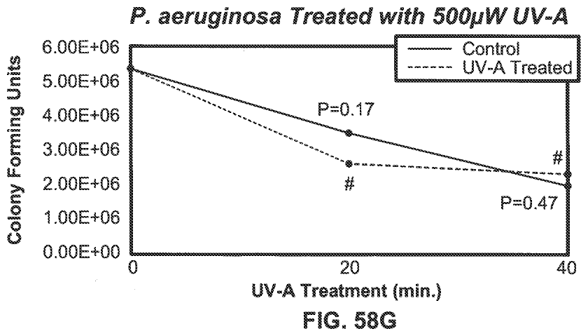

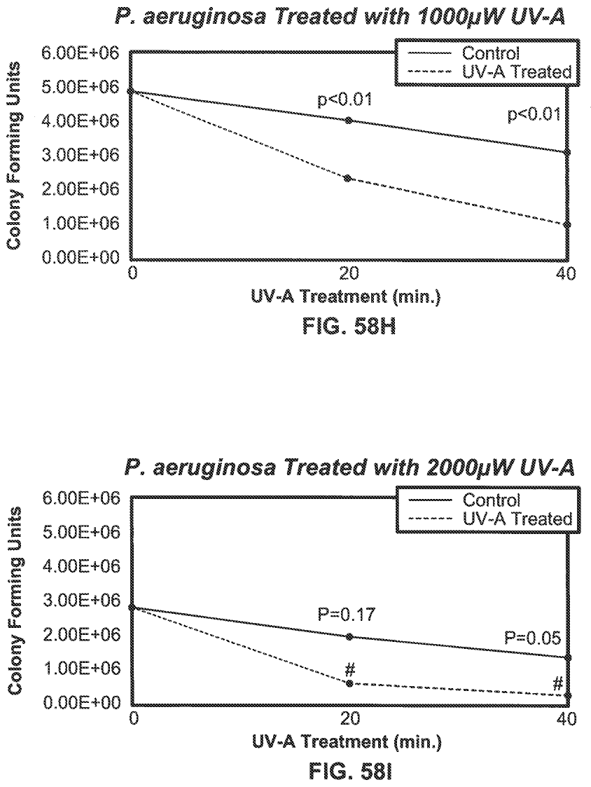

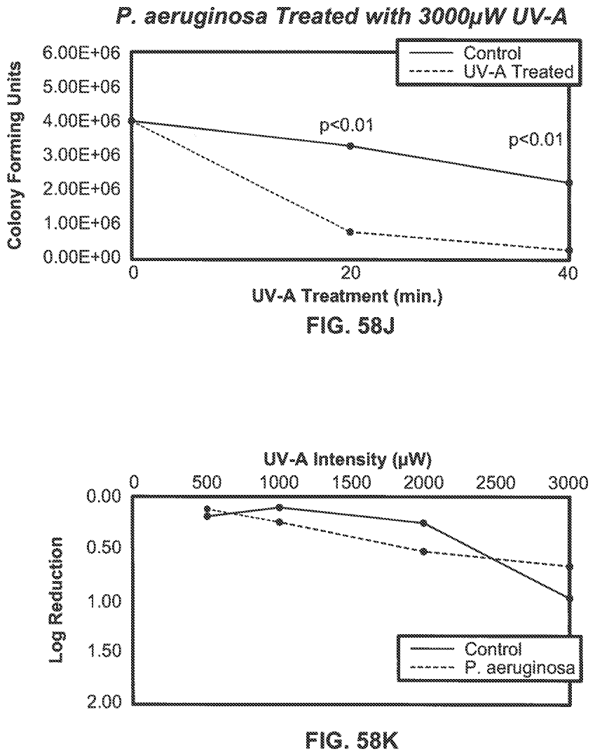

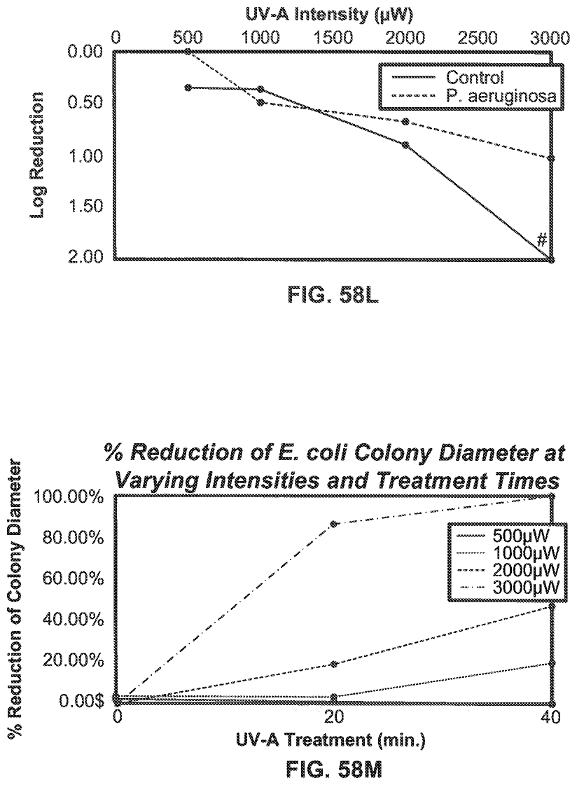

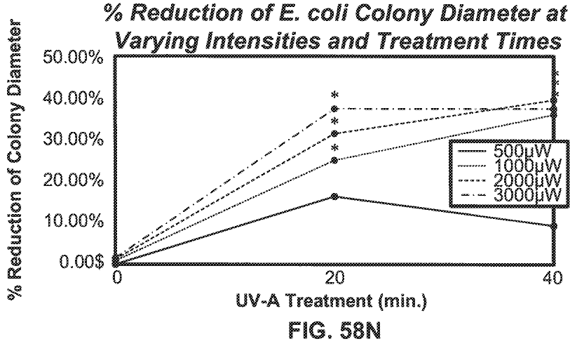

Finally, experiments were conducted to test the effects of LED narrowband UVA (345 nm peak wavelength) on E. coli and Pseudomonas aeruginosa. In these experiments, this specific wavelength of UVA resulted in a significant reduction in bacterial cells as shown in FIGS. 58A-58N. For instance, FIG. 58A illustrates a picture of a bacterial colonies in petri dishes, and the pattern of disappearance of the colony around the site of application of the LED light at 20 and 40 minutes.

FIGS. 58B-58F illustrate graphs showing the change in colony forming units (CFUs) of E. coli over time when UVA light with a peak wavelength of 345 nm is applied at various intensities. As illustrated most of the bacteria were eliminated by 40 minutes with an intensity of 2000 uW (FIG. 58D) and most of the bacteria were eliminated by 20 minutes with an intensity of 3000 uW (FIG. 58E and FIG. 58F). When the same light was applied at 500 uW and 1000 uW of intensity, there was significant reduction of CFUs by 40 minutes, but only by about half (FIG. 58C and FIG. 58B).

FIGS. 58G-58J illustrate graphs showing the change in colony forming units (CFUs) of P. aeruginosa over time when UVA light with a peak wavelength of 345 nm is applied at various intensities. As illustrated, treatment with an intensity of 1000 uW, 2000 uW and 3000 uW showed significantly greater reduction in CFUs compared to control (FIG. 58H, FIG. 58I and FIG. 58J), and most of the bacteria were eliminated by 20 minutes with an intensity of 2000 uW and 3000 uW (FIG. 58I and FIG. 58J).

FIGS. 58K-58L illustrate growth curves comparing the logarithmic reduction of P. aeruginosa at various intensities at 20 minutes and 40 minutes respectively. FIG. 58M illustrates growth curves showing the reduction of a E. coli colony diameter at various intensities and treatment times. FIG. 58N illustrates growth curves showing the reduction of a P. aeruginosa colony diameter at various intensities and treatment times.

Examining the effect of light intensity on the reduction of E. coli and P. aeruginosa, there was a dose response effect on both bacterial levels and colony size (FIGS. 58B-58N). The ideal UVA intensity to impact bacteria appeared to be between 2000 and 3000 μW/cm2 when using a narrowband LED with a peak wavelength of 345 nm, and in some examples may depend on the bacteria or pathogen type and species, and other factors as disclosed herein.

### Example 3: Safety Data

For the assessment of the safety of UVA on mammalian cells, three experiments were conducted. The first was the exposure of UVA to HeLa cells in culture. HeLa cells were added to DMEM cell culture medium (Gibco, Waltham, Mass.) plus 10% Bovine serum (Omega Scientific, Tarzana, Calif.) and 1×Antibiotic-Antimycotic (100× Gibco) in 60×15 mm cell culture dishes (Falcon) and incubated at 37° C. (5% CO₂) for 24 hours to achieve 1,000,000 to 1,800,000 cells per plate. At this point cells were exposed to UVA LED light (1800 μW/cm²) for 0 (control), 10, or 20 minutes. After 24 hours, cells were removed by 0.05% Trypsin-EDTA (1×) (Gibco), stained with Trypan blue (Trypan Blue 0.4% ready to use (1:1) (Gibco)) and quantitated by automated cell counter (Biorad T20, Hercules, Calif.). In a similar experiment, the LED UVA light was used at a higher intensity (5000 μW/cm²) for 20 minutes. Once again, HeLa cells were quantitated at 24 hours following UVA exposure.

Further, the safety of UVA was also studied in two human respiratory cell types. These included alveolar (ATCC A549) and primary ciliated tracheal epithelial cells (HTEpC) (PromoCell, Heidelberg, Germany). For each cell line, 250,000 cells were plated and grown for 48 hours in DMEM until the cell count per plate was approximately 750,000. At this point, cells were exposed to UVA (2000 μW/cm²) for 0 (control) or 20 minutes (treated), and cell counts were obtained at 24 hours later.

The levels of 8-hydroxy-2′-deoxyguanosineis (8-OHdG) was also analyzed in the DNA of cells treated with UVA. 8-OHdG is widely accepted as a sensitive marker of oxidative DNA damage and oxidative stress. DNA was extracted with the AllPrep DNA/RNA/Protein Mini Kit (Qiagen) following manufacturer's instructions. The levels of 8-OHdG was detected using the EpiQuik™ 8-OHdG DNA Damage Quantification Direct Kit following manufacturer's instructions (Epigentek, Farmingdale, N.Y.). For optimal quantification, the input DNA amount was 300 ng, as the basal 8-OHdG is generally less than 0.01% of total DNA (Epigentek, Farmingdale, N.Y.).

Wild type 129S6/SvEv mice (n=20, female=10) and BALB/cJ mice (n=10, female=5) were used for UVA light safety tests. All animals were anesthetized prior the procedure. Prior to the UVA light treatment, animals were placed in an induction chamber containing isoflurane anesthetic gas (1-5%). The carrier gas for isoflurane was compressed oxygen (100% oxygen). Once the respiratory rate had slowed (approximately one breath per second), the animals were removed from the induction chamber and maintained under sedation using a nose cone anesthesia (1-2% isoflurane). The depth of anesthesia was confirmed by lack of response to toe pinch.

Under anesthesia, customized rods (D=4 mm, L=40 mm) were introduced anally up to the splenic flexure. The same procedure was applied to the control group using an identical but unlit rod. Same light source and measurement equipment were used as described for liquid culture experiments.

In the first experiment, 5 BALB/cJ mice underwent colonic UVA exposure (2,000 μW/cm²) for 30 minutes as compared to 5 mice treated with the same technique with an unlit optic rod.

In the second experiment, 10 12956/SvEv mice underwent 20 minutes per day of colonic UVA exposure (3,000-3,500 μW/cm²) for 2 two consecutive days as compared to 10 mice (male=5) treated with an unlit rod.

Colon Endoscopic Examination Before and After UVA Light Therapy

A rigid pediatric cystoscope (Olympus A37027A) was used to assess the intestinal mucosa before and after 7 days of UVA exposure. Endoscopy was performed in anesthetized animals. The method of sedation is described above.

The anus was first lubricated with a water-based gel (Astroglide®, BioFilm, Inc., Vista, Calif., USA). The endoscope was then inserted to the splenic flexure, and the colon was insufflated using room air instilled via an endoscopic port. All endoscopies were recorded and blindly interpreted by two gastroenterologists with expertise in animal model endoscopies. Endoscopic appearances were analyzed based on perianal examination, transparency of the intestinal wall, mucosal bleeding, and focal lesions.

At day 14, control and treated mice were euthanized, and swiss-roll preparations of the entire colon were performed. Briefly, the entire colon was removed and rinsed in a modified Bouin's fixative solution (50% ethanol/5% acetic acid in dH2O). Using scissors, the colon was opened longitudinally along the mesenteric line and rinsed briefly in a Petri dish containing 1×PBS. The luminal side was identified and Swiss rolling of the opened tissue was performed. Once the entire colon length was rolled, the colon was carefully transferred to a tissue-processing/-embedding cassette. The cassette was placed in 10% buffered formalin overnight at room temperature, after which paraffin sections of the colon were cut, stained with hematoxylin and eosin (H&E), and assessed by a blinded pathologist (SS).

Data for bacterial counts between groups were not normally distributed and were therefore compared using non-parametric tests (Mann Whitney U test). Other quantitative data were compared by t-test using GraphPad Prism 7 (GraphPad, San Diego, Calif.).

### Results

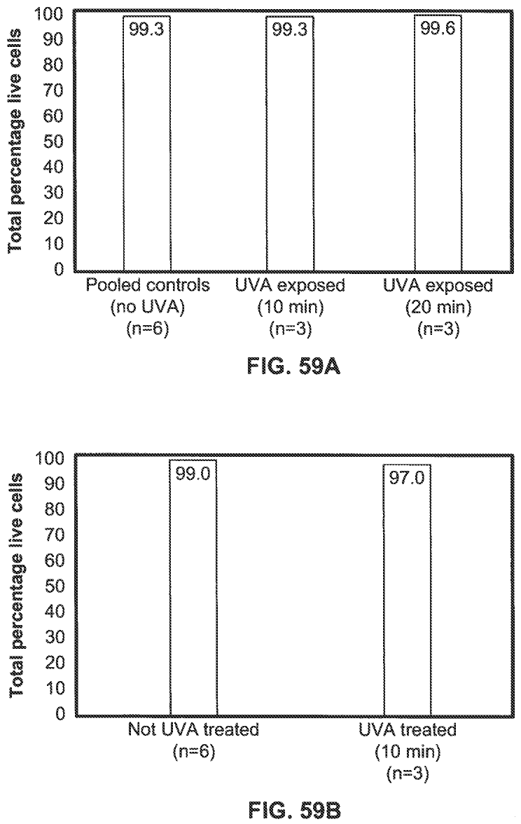

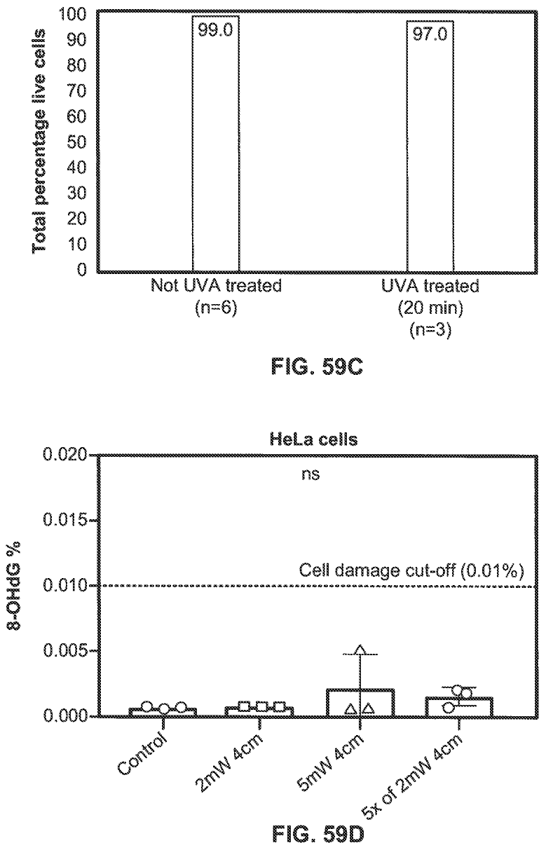

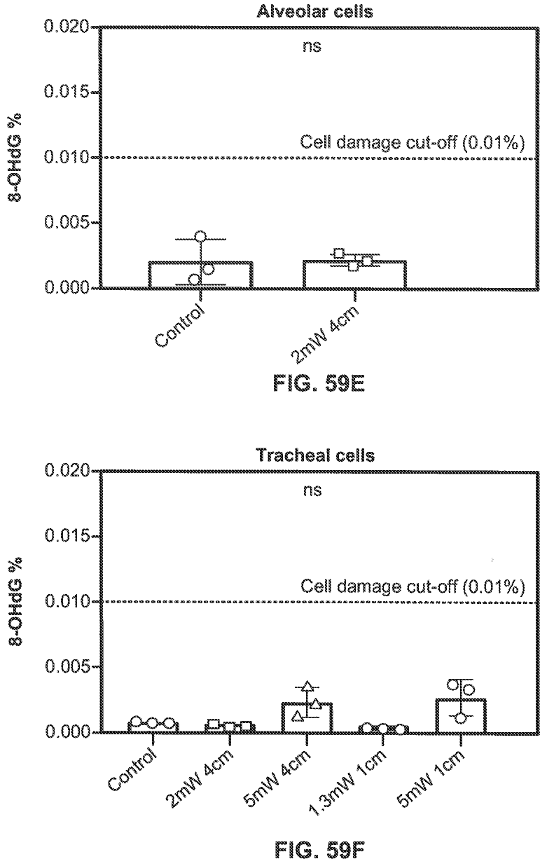

Overall, based on cell growth over time, LED UVA appeared safe in the mammalian cells tested (HeLa, alveolar A549 and primary tracheal cells). All plates demonstrated continued cell growth, regardless of UVA exposure, with 1.5 to 2 times the number of cells per plate compared to controls, indicating robust ongoing replication. In the case of HeLa cells, UVA did not affect the number of live cells at 24 hours when compared to unexposed controls (P=0.99 and P=0.55 for 10 min and 20 min of 2000 μW/cm² UVA, respectively) as shown in FIG. 59A. Higher intensity UVA (5000 μW/cm²) did not affect the growth of HeLa cells as shown in the bar graph depicted in FIG. 59B. Similar findings were also seen with alveolar cells at 2000 μW/cm² for 20 min (P=0.99) as shown in FIG. 36C. Finally, ciliated epithelial cell growth was also unaffected by UVA after 20 minutes of exposure to 1000 μW/cm² and to 2000 μW/cm².

Moreover, exposure to UVA did not cause DNA damage in any cell line analyzed, and the levels of 8-Oxo-2′-deoxyguanosine (8-OHdG) in cells treated with narrow-band LED UVA was similar to controls not exposed to UVA (P<0.05) as shown in FIG. 59D (HeLa cells), FIG. 59E (Alveolar cells), and FIG. 59F (Tracheal cells). Higher intensity LED UVA (5000 μW/cm²) appeared to increase the levels of 8-OHdG (P=0.07) but the percentage of 8-OHdG remained well below the generally accepted threshold of 0.01% of the total DNA.

UVA Light Exposure is not Associated with Endoscopic or Histologic Injury

To assess the safety of UVA therapy on internal visceral cells and tissues, two different wild-type strains of mice were exposed to intracolonic wide-spectrum UVA light using optical rods designed to homogenously side-emit broad-spectrum UVA. Only the left side of the colon up to the splenic flexure were exposed to UVA light; hence, the unexposed right side served as a self-control. In the first experiment, under anesthesia, 5 mice underwent colonic UVA exposure (2,000 μW/cm2) for 30 minutes as compared to 5 mice treated with the same technique with an unlit optic rod.

In the second experiment, 10 mice (129S6/SvEv, male=5) underwent 20 minutes per day of colonic wide spectrum UVA exposure (3,000-3,500 μW/cm²) for 2 two consecutive days as compared to 10 mice (male=5) treated with an unlit rod. No perforation, bleeding or fatalities were seen in any of the experiments. The mouse colonoscopy images show no change before and after UVA exposure.

In both experiments, endoscopic evaluation of mice before and after UVA administration demonstrated no macroscopic evidence of mucosal erythema, friability, ulceration or bleeding. Assessed by a blinded pathologist (SS), no chronic/acute inflammation, cystitis, crypt abscesses, granulomata, ulceration or dysplasia was seen on examined full-thickness colonic specimens exposed to wide spectrum UVA as compared controls and untreated segments of the colon.

### Additional Safety Data and Results

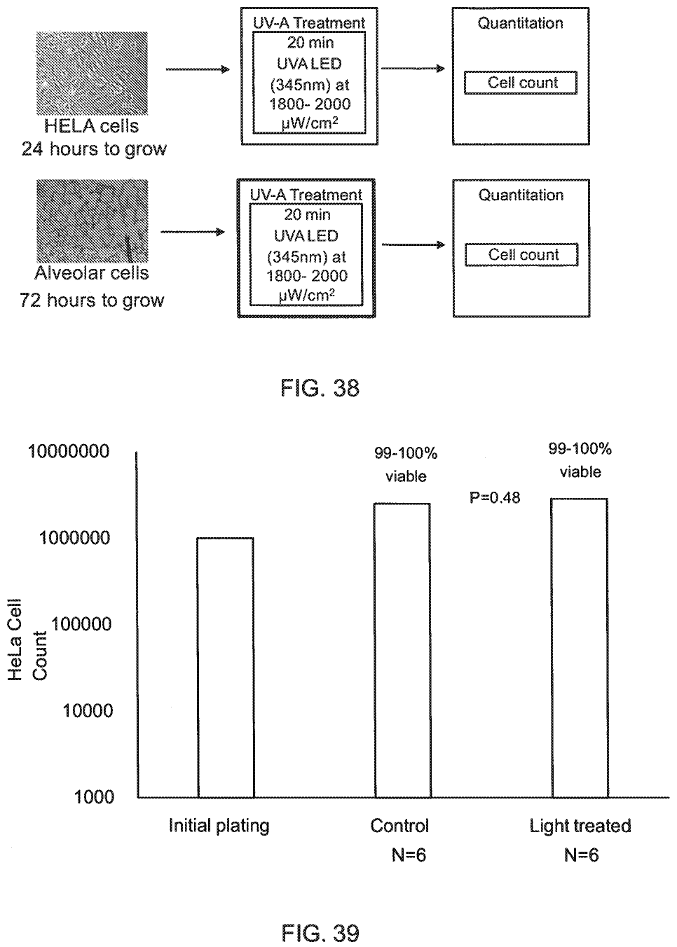

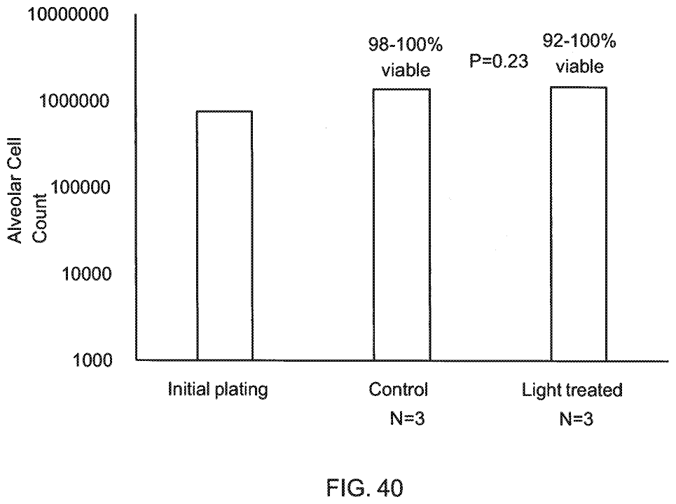

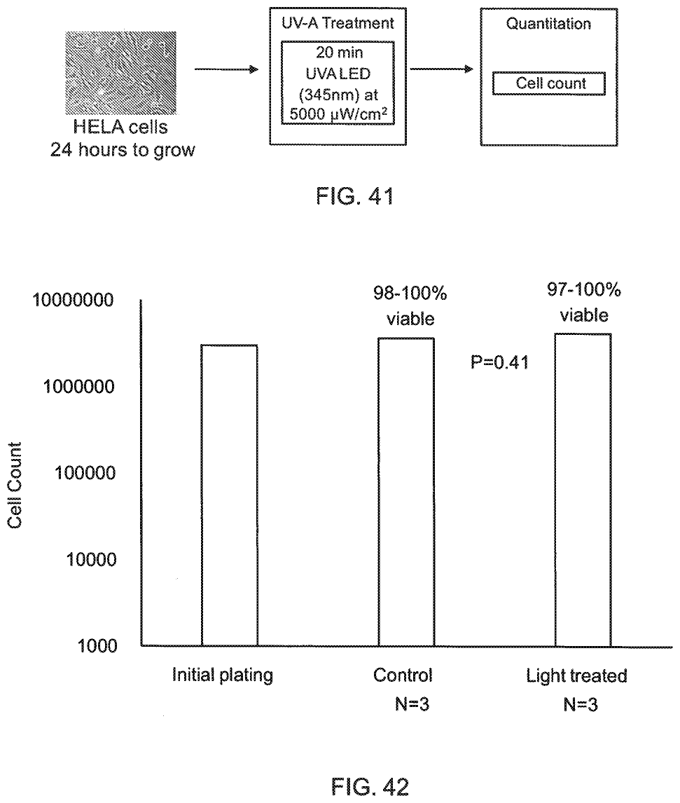

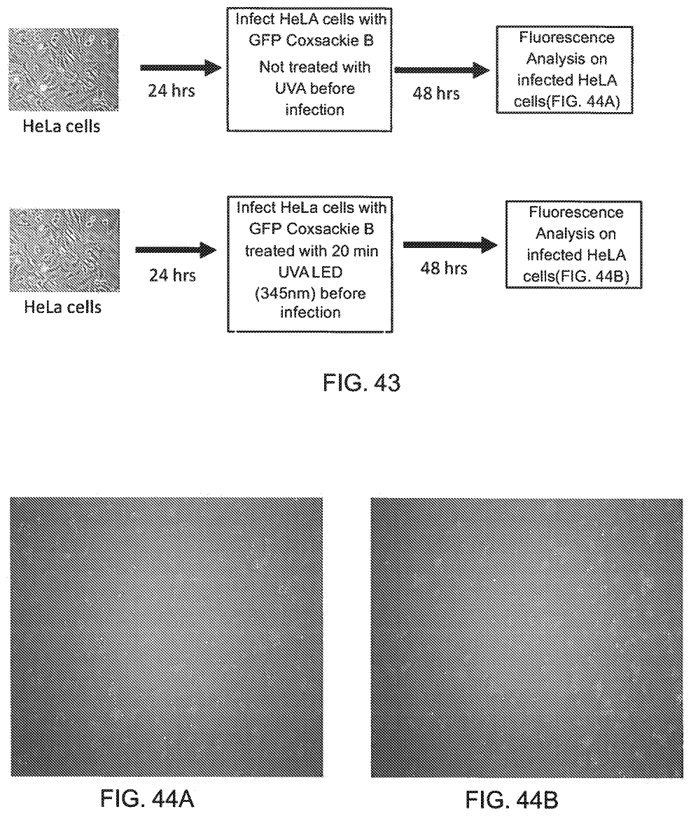

Turning to FIG. 38, HeLa cells were grown for 24 hours, treated with UVA light (1800-2100 μW/cm² up to 20 min) and quantitated by cell count. Also, as shown at FIG. 38, Alveolar cells were grown for 72 hours, treated with UVA light (1800-2100 μW/cm² up to 20 min) and quantitated by cell count. HeLa cell count and Alveolar cell count are shown at FIGS. 39 and 40. When treated with UVA light, HeLa cells and Alveolar cells had 99-100% viability and 92-100% viability respectively comparable to control cells not treated with UVA light. Similarly, when higher intensity of UVA light was tested (5000 μW/cm²) on HeLa cells (FIG. 41), UVA light treated HeLa cells showed 97-100% viability compared to 98%-100% viability of control (HeLa cells not treated with UVA). This further highlights the safety of UVA treatment, particularly at intensity, wavelength, and duration that is effective for anti-viral treatment.

### RNA Virus Experimental Data

Additionally, the disclosed systems and methods were utilized to obtain experimental data in treating various RNA viruses with UVA light. Accordingly, the data illustrates that UVA light emitted from an LED with a peak wavelength of 340 nm, can kill RNA viruses like Coxsackievirus. For instance, the Hela cells infected with Coxsackievirus survived when this UVA treatment was applied, but did not survive when there was no UVA light treatment applied after infection. Furthermore, the experimental data demonstrated only a 15% loss of UVA light once it passed through an ETT.

In late December 2019, an outbreak of a novel coronavirus disease (SARS-CoV-2 or COVID-19; previously known as 2019-nCoV) was reported in Wuhan, China. COVID-19 is a viral infection that replicates efficiently in the upper respiratory tract. As part of the mechanism of action, the virus infects ciliated tracheal epithelial cells, which then slough off and compromise alveolar function. Secondary bacterial infections have also been noted, and both of these processes can lead to further inflammation, acute respiratory distress syndrome (ARDS), and ultimately, death. It is estimated that 10-15% of those infected have a severe clinical course and about 5% become critically ill, requiring mechanical ventilation for failure of the respiratory and other organ system. The case fatality rate of COVID-19 has been estimated to range from 0.5% to 9.5%, although these estimates are confounded by preferential testing of symptomatic patients and a lag time of up to 14 days for symptom presentation. Death is thought to be due to respiratory failure in the setting of ARDS and/or secondary infections including ventilator associated pneumonia (VAP).

Ventilator-associated pneumonia (VAP) may develop in intensive care unit (ICU) patients who are mechanically ventilated for at least 48 hours, which is common in COVID-19 patient. The incidence of VAP ranges broadly from 5% to 67%, depending on the diagnostic criteria used and patient population studied. Causative organisms include Enterobacteriaceae (25%), Staphylococcus aureus (20%), Pseudomonas aeruginosa (20%), Haemophilus influenza (10%), and streptococci (13). Multi-drug resistant bacteria are more common among late-onset cases. Mortality attributed to early-onset VAP is thought to be approximately 6% while that for late-onset VAP is 10%.

Currently, there is no treatment for COVID-19 and conventional means to reduce secondary infections in mechanically ventilated patients have proven insufficient to date. A safe and effective broad antiviral and antibacterial approach to these patients would potentially reduce viral burden, secondary infection and VAP, time on mechanical ventilation, and death due to respiratory failure.

As disclosed herein, ultraviolet (UV) light has antibacterial properties. UVC (110-280 nm) is widely used for industrial sterilization, but has harmful effects on human DNA. External UVA (320-400 nm) and UVB (280-320 nm) devices have FDA-approved indications to treat human diseases such as psoriasis, eczema, and skin lymphoma. These wavelengths penetrate the mucosal and submucosal tissue. Of the three spectrums, UVA appears to cause the least damage to mammalian cells. Presently, there are no studies showing the effects of an internal application of UVA light for bacterial or viral infections.

Accordingly, disclosed is experimental data illustrating the effects of broad and/or narrow band UVA for the treatment of common bacterial pathogens known to be associated with VAP. Additionally, disclosed is data that demonstrates on the effects of a specific wavelength of UVA on group B coxsackievirus and coronavirus 229E. Finally, further data demonstrates the safety of UVA exposure for mammalian cells and in vivo epithelial cells.

### Example 4: Coxsackievirus

Coxsackievirus Sample Obtainment and Infection into Cells

Recombinant coxsackievirus B (pMKS1) expressing enhanced green fluorescent protein (EGFP-CVB) plasmid was linearized using ClaI restriction enzyme (ER0142, Thermo Fisher) and linearized plasmid was purified using standard phenol/chloroform extraction and ethanol precipitation. Viral RNA was then produced using mMessage mMachine T7 Transcription kit (AM1344, Thermo Fisher). Viral RNA was then transfected into HeLa cells (˜80% confluency) using Lipofectamine 2000 (11668027, Thermo Fisher). Once cells exhibited ˜50% cytopathic effect, cells were scraped and the cell/media suspension was collected. This mixture was then subjected to three rounds of rapid freeze-thaw cycles and centrifuged at 1000×g for 10 minutes to clarify media of cellular debris. Supernatant was used as passage 1 viral stock. The passage 1 viral stock was then overlain onto separate HeLa cells (˜80% confluency) to expand the stock into passage 2 viral stock which was used for subsequent experiments.

UVA Treatment on HeLa Cells Infected with Group B Coxsackievirus

HeLa cells were used for four different experiments with enhanced green-fluorescent protein (EGFP)-expressing group B coxsackievirus (EGFP-CVB). In the first experiment, HeLa cells (253,000 per plate) (n=12 plates) were cultured for 24 hours. Half of the EGFP-CVB aliquots were exposed to LED UVA (2000 μW/cm²; peak wavelength of 340 nm) for 20 minutes while the other was not exposed; HeLa cells were then infected with either UVA-exposed or UVA-unexposed virus (MOI=0.1). Six hours later, the supernatant was removed, and the cells were washed twice with 1×sterile PBS (pH=7.0). New DMEM media was added. Plates that were infected with UVA-exposed virus received an additional 20 minutes of UVA (2000 μW/cm²) exposure. Dead cells in the supernatant were collected and quantified 24 hours later. Six plates (3 each of UVA and non-UVA treated) were assessed for live cells. Of the remaining six plates, the 3 plates which had initially been exposed to UVA with a peak wavelength of 340 nm were then exposed to an additional 20 minutes of UVA (2000 μW/cm²). After an additional 24 hours, dead and live cells counts were obtained from the remaining plates.

HeLa Cell Pre-Treatment with UVA on Group B Coxsackievirus Infection

In the second experiment, HeLa cells (235,000 cells) were plated and then incubated in DMEM for 24 hours. The plates were then divided into unexposed controls (n=3) and exposed to LED UVA (2000 μW/cm²; peak wavelength of 340 nm) for 20 minutes (n=3). After another 24 hours, all plates were infected with EGFP-CVB (M01=0.1). After an additional 24 hours, cells were counted as previous described.

Pre-Treatment of Group B Coxsackievirus with UVA on HeLa Cell Infection

In the third experiment, HeLa cells were cultured for 24 hours and then infected with EGFP-CVB (MOI=0.1). Just prior to infection, half of the EGFP-CVB aliquots were exposed to LED UVA (2000 μW/cm²; peak wavelength of 340 nm) and the other half remained unexposed. Twenty-four hours later, a viable cell count was obtained.

Long-Term UVA Treatment of HeLa Cells During Ongoing Group B Coxsackievirus Infection

In this experiment, 250,000 HeLa cells were plated. At 24 hours, cells were divided into three groups. In the first group, cells were infected with EGFP-CVB (MOI=0.1). These cells served as positive infected controls. In group 2, HeLa cells were infected with UVA-treated (2000 μW/cm2 for 20 min; peak wavelength of 340 nm) EGFP-CVB (MOI=0.1) and 6 hours later the infected cells were treated with UVA (2000 μW/cm²; peak wavelength of 340 nm) for 20 minutes followed by 4 additional treatments including day 2 for 20 minutes twice 8 hours apart, and day 3 twice for 20 minutes, 8 hours apart. Group 3 was not infected with EGFP-CVB but was treated 5 times with UVA at the same timepoints used for group 2. This was the non-infected positive control to demonstrate safety of UVA. In all conditions, imaging and cell counts were obtained.

UVA Treatment on Alveolar (A549) Cells Infected with Group B Coxsackievirus

Ideal timepoints of cell death from infection were determined in preliminary experiments with alveolar cells to be 48 hours after infection. In this study, 200,000 alveolar cells were plated and counted at 48 hours (cell count of 754,000). Alveolar cells were then infected with EGFP-CVB (MOI=0.1). Twenty-four hours after infection, the alveolar cell plates were exposed to LED UVA (2000 μW/cm²; peak wavelength of 340 nm) for 0 (control) or 20 minutes (treated) and this was repeated every 24 hours for three days with imaging and cell counts at 96 hours post-infection.

### Results

UVA Pre-Treatment of Group B Coxsackievirus Only Prior to Infection of HeLa Cells does not Mitigate Infection

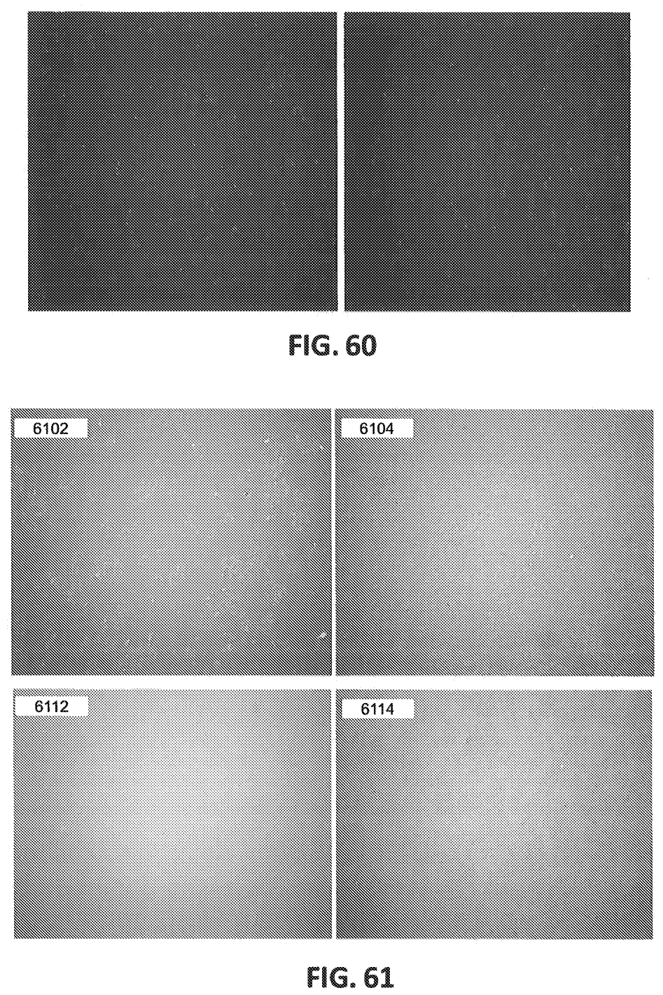

In this experiment, half of the plates with HeLa cells were transfected with EGFP-CVB and the other half were treated with EGFP-CVB that was exposed to ˜2000 μW/cm² LED UVA light with a peak wavelength of 340 nm for 20 minutes. The effect on infection rates at 24 hours were not different between groups (FIG. 60).

UVA Pre-Treatment of HeLa Cells Prior to Group B Coxsackievirus Infection does not Mitigate Viral Effects

In this experiment, half of the plates with HeLa cells were left untreated and the other half were pre-treated with ˜2000 μW/cm²LED UVA; peak wavelength of 340 nm for 20 minutes with no further UVA treatment. EGFP-CVB was added to both groups. Both groups were equally infected, suggesting that treating HeLa cells before infection did not influence the infection rate.

UVA Treatment after Infection with Group B Coxsackievirus Reduced Viral Effect on HeLa Cells

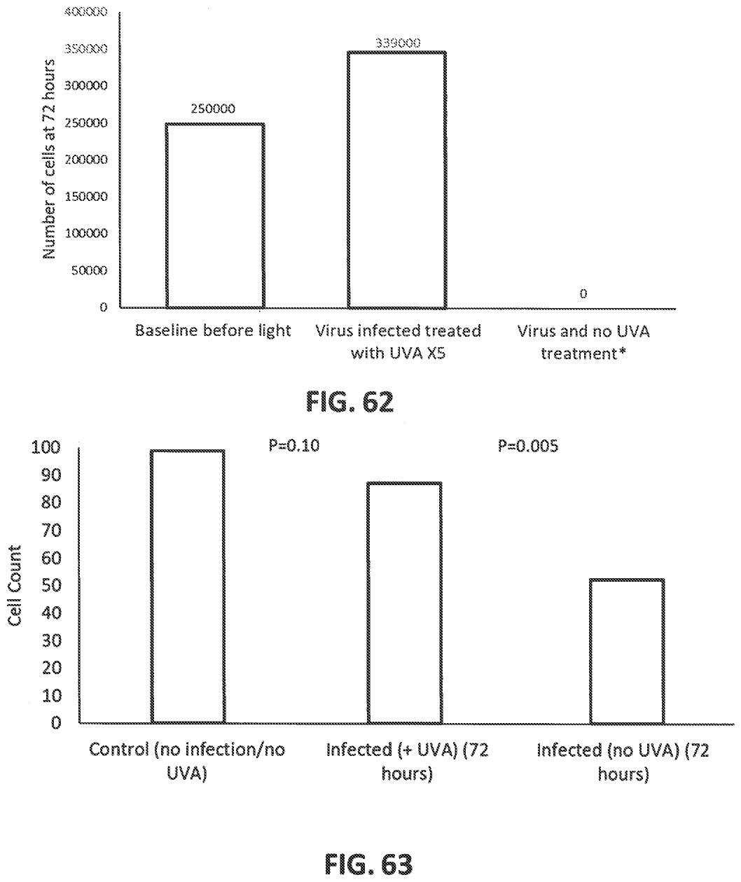

In this study, UVA was applied after the HeLa cells were infected with EGFP-CVB. Treated cells were exposed to 2000 μW/cm²LED UVA with a peak wavelength of 340 nm at 6 hours post-infection, then twice daily for two additional days, with cell counts at 72 hours post-infection. This was compared to infected but untreated controls. In the treated group, UVA light prevented cell death from EGFP-CVB, with increased cell counts to 339,333±60,781 at 72 hours as shown in the bar graph depicted in FIG. 62 (also shown in FIG. 61), compared to no live cells remaining on plates at 48 and 72 hours in untreated controls. Importantly, a third group of HeLa cells that were not infected but received UVA exposure at the same time intervals showed normal cell proliferation, with a cell count of 2,413,333±403,773 at 72 hours.

FIG. 61 shows effects of NB-UVA exposure on HeLa cells transfected with group B coxsackievirus. Images 6102 and 6104 show cells at 24 hours after transfection: Reduced number of adherent cells in UVA-unexposed plates (left panel 6102, percent dead cells in supernatant=67.5±11.0%) compared with UVA-exposed plates (right panel 6104, percent dead cells in supernatant=16.1±5.8%) (P=0.002). (Magnification=4×, overlay of green light and bright field). Images 6112 and 6114 show cells at 48 hours after transfection (Left panel 6112 shows no remaining live cells (unexposed to UVA)). Right panel 6114 shows survival of UVA-exposed cells (Magnification=4×, overlay of green light and bright field).

The Effect of UVA Treatment on Alveolar (A549) Cells Infected with Group B Coxsackievirus

In alveolar cells infected with EGFP-CVB, cell death was far less than that seen in HeLa cells. At 96 hours post infection, there was clear and widespread infection of cells in the control group. Alveolar cells treated with LED UVA with a peak wavelength of 340 nm also demonstrated infection, but visual assessment suggested a lower rate of infection, with far fewer cells producing viral EGFP signals. In addition, viable cell counts appeared to be higher in the UVA treated group when compared to the untreated group.

Additional Experimental Data with GFP Tagged Coxsackie Virus B (EGFP-CVB)

Referring to FIG. 43, fluorescence microscopic analysis was performed on HeLa cells that were transfected with EGFP-CVB, where the EGFP-CVB was treated with UVA (20 min, 345 nm peak wavelength) prior to transfection. Control group included HeLa cells transfected with untreated EGFP-CVB. Results of the fluorescence microscopy analysis are shown at FIGS. 44A and 44B (control). As evidenced, UVA did not have a significant effect on extracellular coxsackie virus. That is, pre-treated and untreated GFP-CVB show similar rates of infection of HeLa cells as evidenced by GFP fluorescence imaging.

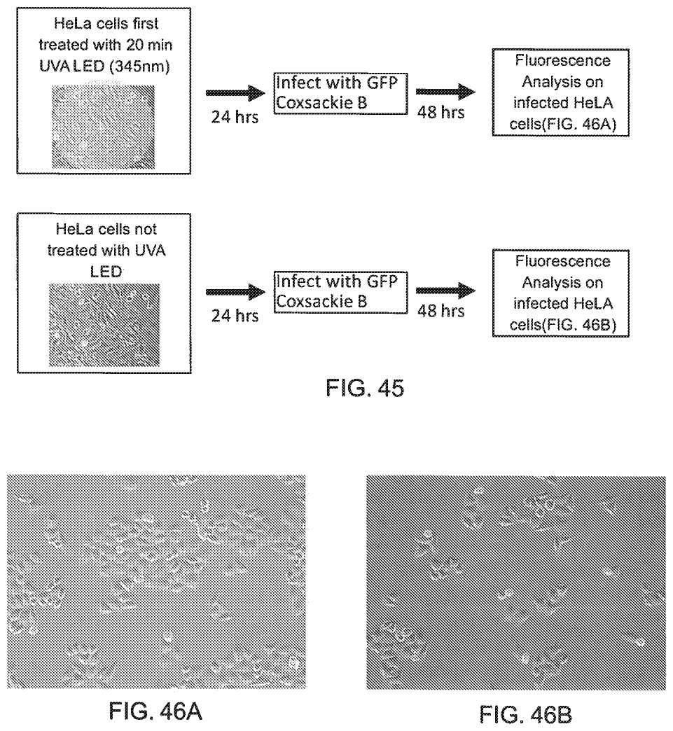

Further, in another experiment, shown at FIG. 45, HeLa cells were pre-treated with UVA prior to transfection with GFP-CVB. Control group included untreated HeLa cells. 48 hours after transfection, fluorescence microscopy analysis was performed. As evidenced by bright-field and fluorescence merged images in FIGS. 46A and 46B, pre-treatment of UVA on HeLa cells did not have a significant impact on infection rates compared to control (untreated HeLa cells).

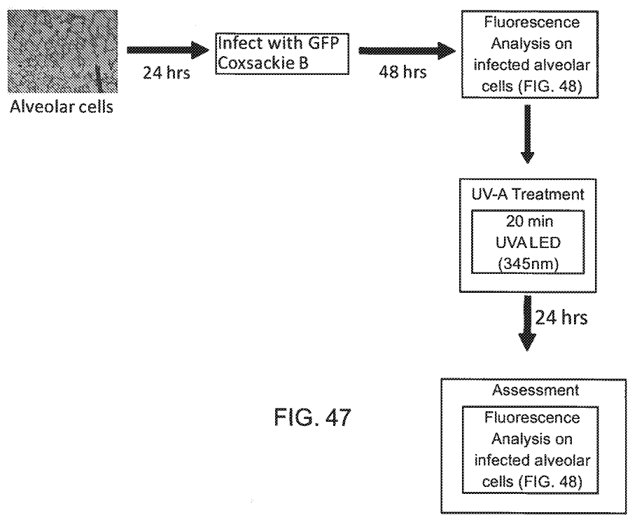

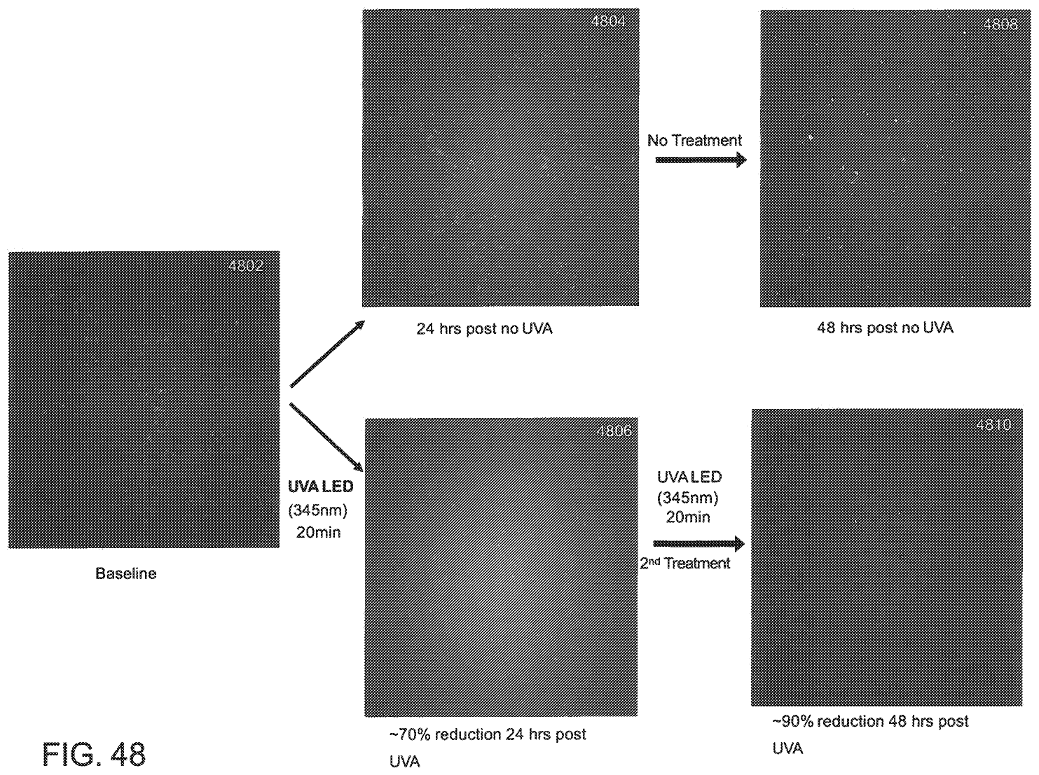

Assessment of UVA treatment by fluorescence microscopy analysis of Alveolar Cells Infected with GFP-CVB (FIGS. 47 and 48)

Alveolar Cells Cultured for 24 Hours were Transfected with GFP-CVB, and fluorescence microscopy was performed after 48 hours to establish baseline (image 4802). The transfected cells were then treated with UVA and imaged at 24 hours (image 4806) and 48 hours (image 4810) post transfection. Control group included GFP-CVB transfected cells but without UVA treatment. The control group without UVA treatment was also imaged at 24 hours (image 4804) and 48 hours (image 4808) post transfection. As can be seen at the images at FIG. 48, UVA treatment resulted in approximately 70 reduction of GFP-CVB infection at 24 hours and approximately 90 reduction of GFP-CVB at 48 hours. UVA treatment was performed with UV LED having peak wavelength at 345 nm and for 20 minutes.

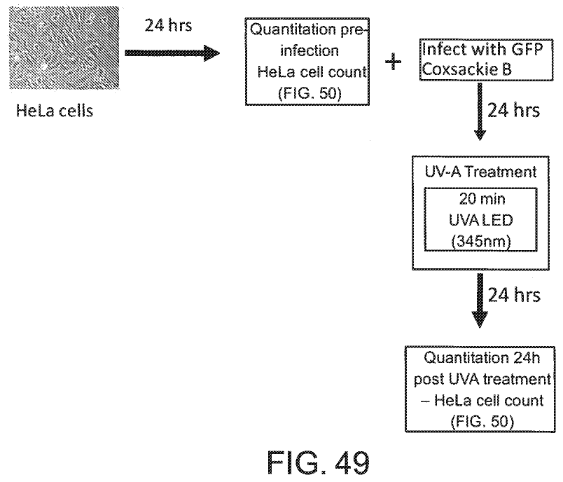

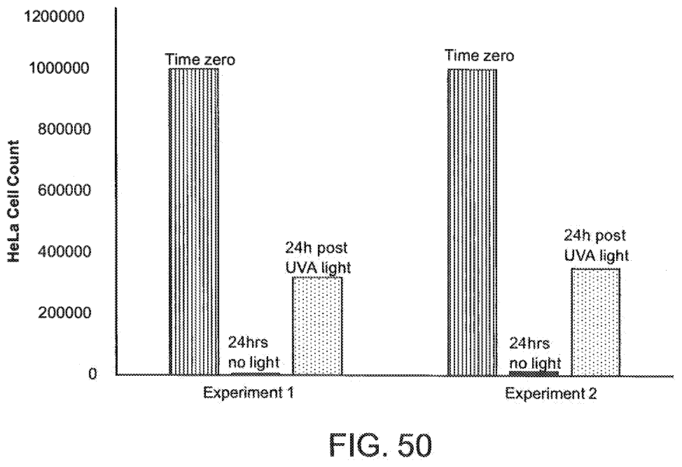

Assessment of UVA Treatment by Quantitative Analysis of HeLa Cells Infected with GFP-CVB (FIGS. 49 and 50)

HeLa cells cultured for 24 hours were counted prior to transfection with GFP-CVB (time zero at FIG. 50). After transfection, HeLa cells were cultured for 24 hours with GFP-CVB. At 24 hours, UVA treatment was performed on one group. Control group included GFP-CVB transfected HeLa cells without UVA treatment. A final cell count was performed on UVA treated and untreated GFP-CVB transfected HeLa cells. As shown at FIG. 50, HeLa cell survival increased significantly with UVA treatment. Similar to above experiments, UVA treatment was performed with UV LED having peak wavelength at 345 nm for a duration of 20 minutes.

### Example 5: Coronavirus

In another example, coronavirus infected ciliated tracheal epithelial cells (HTeC) were treated with UV light as disclosed below.

Ciliated tracheal epithelial cells (Promocell, Heidelberg, Germany) were plated (135,000 per plate) into three groups. One group was infected with coronavirus 229E (Cov-229E) (50 uL per plate). In the other group, just prior to infection, coronavirus 229E was treated with LED UVA with a peak wavelength of 340 nm (2000 μW/cm²) for 20 minutes. A third group received no infection or UVA. After infection, the cells were treated with UVA (4 cm distance with 2000 μW/cm² at surface of plate with a peak wavelength of 340 nm) for 20 minutes daily. Plates were imaged at 16, 72 and 96 hours, and cell counts were obtained at 72 and 96 hours after infection.

UVA to Salvage Already Infected (with Coronavirus 229E) Ciliated Tracheal Epithelial Cells

In this experiment, plates of ciliated tracheal epithelial cells (HTeC) were infected with Cov-229E as above. At 24 hours, plates were divided into two groups. Group 1 was left to continue the infection. In group 2, plates were treated with UVA with a peak wavelength of 340 nm (4 cm distance with 2000 μW/cm² at surface of plate) for 20 minutes. At 48 hours, plates were imaged, and viable cell counts were obtained.

UVA to Treat Coronavirus Infected Ciliated Tracheal Epithelial Cells at Close Range

In the anticipation of an endotracheal device using UVA technology, another experiment was conducted identical to the above experiments using lower intensity of light (1300 μW/cm² at the surface of the plate from only 1 cm distance) for 20 minutes daily. This would be the anticipated distance between a light catheter and the tracheal cells in the ventilated patient from the inside of an endotracheal tube.

Level of Coronavirus in Cells with or without UVA Treatment

AllPrep DNA/RNA/Protein Mini Kit (Qiagen) was used to extract total protein from cell samples. Proteins were loaded into a Bolt 4-12% Bis-Tris gel (NW04122 Thermo Fisher) and transferred onto a Biotrace NT nitrocellulose membrane (27376-991, VWR). Total proteins were stained with Ponceau S solution (P7170, Sigma-Aldrich). The membrane was then blocked in blocking solution (tris-buffered saline containing 3% bovine serum albumin (A7030, Sigma-Aldrich) and 0.1% Tween 20 (P1379, Sigma-Aldrich). The membrane was then incubated overnight at 4° C. with either rabbit anti-coronavirus spike protein antibody (1:1000; PA5-81777, Thermo Fisher) or mouse anti-MAVS (mitochondrial antiviral signaling) antibody (1:200; SC-166583, Santa Cruz Biotechnology) diluted in blocking solution. After washing in tris-buffered saline+0.1% Tween 20 (TBS-T), the membrane was then overlain with either horseradish peroxidase (HRP)-conjugated goat anti-rabbit IgG antibody (1:300; 95058-734, VWR) or HRP-conjugated goat anti-mouse IgG antibody (1:300; 5220-0286, SeraCare). The membrane was then washed in TBS-T and subsequently exposed to enhanced chemiluminescence solution (RPN2235, GE Healthcare). Immunoreactive protein bands were imaged using a ChemiDoc Imaging System (Bio-Rad Laboratories, Hercules, Calif. USA).

LED UVA Light Preserves Ciliated Tracheal Epithelial Cells Infected with Coronavirus 229E

Pre-treatment of ciliated tracheal epithelial cells with coronavirus 229E and daily LED UVA (2000 μW/km²; peak wavelength of 340 nm) for 20 minutes was compared to control cells (no UVA and no infection) and cells infected with coronavirus but no UVA exposure. Direct visualization showed definitive changes in cell morphology with infection (no UVA). However, control cells and infected cells treated with daily UVA exhibited similar morphology. At 96 hours, the supernatant was removed and the viable cells (adherent to the plate) were counted. There was no difference in tracheal cell number between control and infected cells treated with UVA. However, there was a marked reduction in viable cells among those infected compared to UVA treated cells (P=0.005) as illustrated in the bar graph shown in FIG. 63.

Interestingly, infected cells treated with LED UVA revealed decreased Cov-229E spike (S) protein (˜130 kDa) when compared to the infected cells not treated. Moreover, the levels infected with Cov-229E and treated with UVA had increased levels of MAVS when compared to cells infected with Cov-229E but not treated with UVA.

Accordingly, the experimental data confirms that UVA light will kill coronavirus 229 E after infecting the epithelial lung tissue, and validates its application in conjunction with ETTs and other devices to irradiate the lung tissues as a treatment for coronavirus infected patients.

Microscopic Analysis of Cell Morphology of Tracheal Cells Transfected with Coronavirus 229E

HTEpC (135,000 cells) were plated into three groups. Group 1 was transfected with CoV-229E (n=3, 50 uL per plate). In group 2, prior to transfection, CoV-229E was exposed to NB-UVA (n=3, 2000 μW/cm2) for 20 min. Group 3 was not exposed to NB-UVA or transfected (n=3). After transfection, the cells were exposed to NB-UVA (4 cm distance with 2000 μW/cm2 at the plate surface) for 20 min daily. Plates were imaged at 16, 36, 72, and 96 hrs, cell viability (live/dead) counts were obtained at 48 and 72 hrs post-transfection. Trypan Blue 0.4% (1:1) (Gibco) was used to determine live/dead cells and cell counts were obtained using an automated cell counter (Biorad T20, Hercules, Calif.). Cells were kept at 37° C. (5% (CO2).

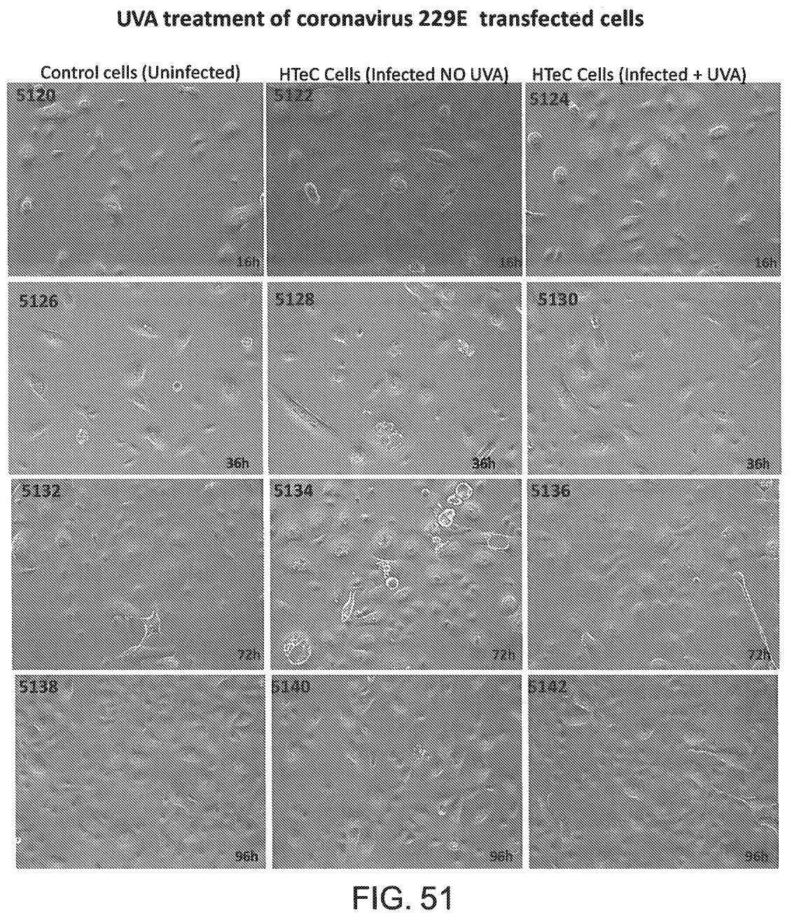

FIG. 51 shows phase contrast images showing the effect of UVA treatment on coronavirus 229E infection of HTeC cells at A) 16 hours, B) 36 hours, C) 72 hours and D) 96 hours post transfection. Left panels images 5120, 5126, 5132, and 5138 show uninfected, untreated control cells; middle panels images 5122, 5128, 5134, and 5140 show cells transfected with coronavirus 229E; and right panel images 5124, 5130, 5136, and 5142 show cells transfected with UVA-treated coronavirus 229E and then treated with UVA. As shown, cells transfected with coronavirus 229E exhibit increasing vacuolation and cell death over time, resulting in decreased cell density. In contrast, transfected and UVA-treated cells remain viable and exhibit similar morphology to controls.

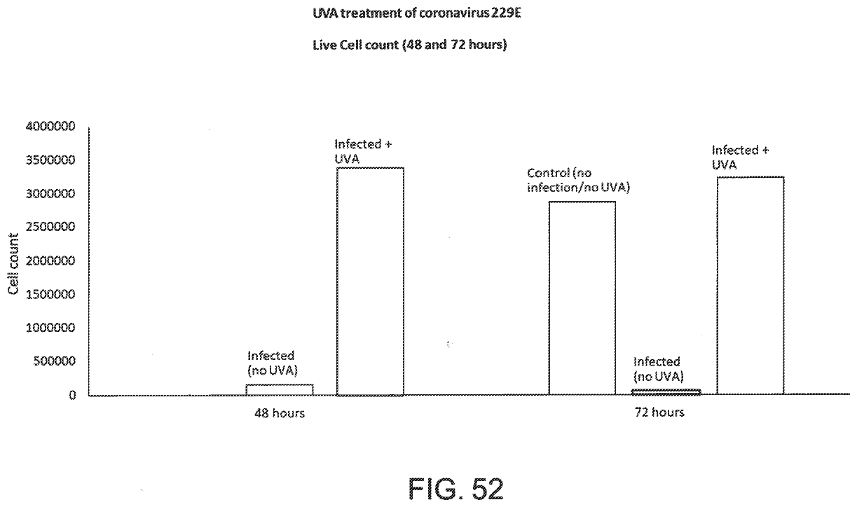

FIGS. 52-54 are bar graphs showing the effect of UVA treatment on coronavirus 229E transfected HTeC cells at 48 hours and 72 hours post-UVA treatment compared to untreated controls. As evidenced in FIGS. 52-54, UVA treatment increases cell viability of coronavirus 229E transfected HTeC cells.

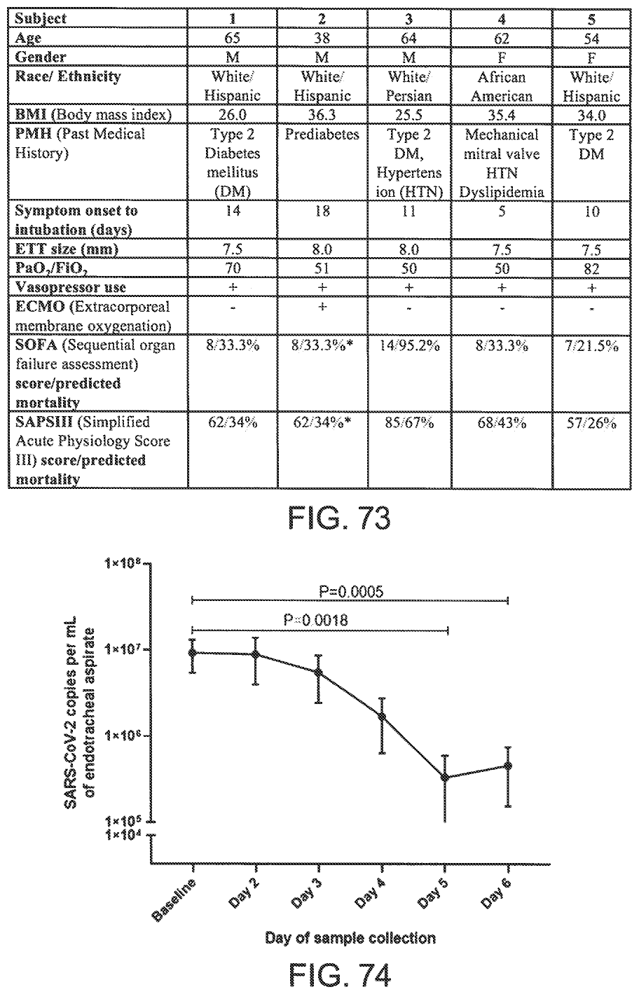

Example in-human-study: Effect of endotracheal UVA light therapy on patients with Severe Acute Respiratory Syndrome Coronavirus-2 (SARS-CoV-2) (FIGS. 73-76).

Ultraviolet-A light was administered via catheter introduced into the endotracheal tube for 20 minutes daily, for 5 days, to newly intubated mechanically ventilated adults with SARS-CoV-2 infection, with an endotracheal tube size 7.5 mm or greater. Pregnant women were excluded. Concomitant therapies were permitted.

Main Outcomes and Measures: The a priori primary measure was respiratory SARS-CoV-2 viral load, obtained from endotracheal aspiration just prior to each treatment and on day 6. Clinical outcomes were assessed through day 30, including the World Health Organization (WHO) COVID-19 10-point ordinal clinical severity scale.

Summary of Results: Five subjects were enrolled (mean age 56.6 yrs, 3 male). At baseline, all subjects scored 9/10 on the WHO clinical severity scale (10=death) with predicted mortality ranging from 21 to 95%. Average log changes in endotracheal viral load from baseline to day 5 and day 6 were −2.41 (range −1.16 to −4.54; Friedman p=0.002) and −3.2 (range −1.2 to −6.77; Friedman p<0.001), respectively. Absolute endotracheal bacterial loads remained unchanged from day 0 to 6. There were no treatment-emergent adverse events, with no changes in oxygenation or hemodynamics during the 20-minute treatments. One subject died 17 days after enrollment due to intracranial hemorrhagic complications of anticoagulation while receiving extracorporeal membrane oxygenation. The remaining subjects survived and scored 2, 4, 5, and 7 at day 30 on the WHO scale at day 30.

In this first-in-human study, endotracheal UVA therapy was safe with a significant reduction in respiratory SARS-CoV-2 viral burden over the treatment period. Details of the method and results are provided below.

Trial Design

In this first-in-human study, 5 subjects were recruited and treated. Inclusion criteria included age over 18 years, positive PCR test result for SARS-CoV-2 on nasal swab, and mechanical ventilation with an endotracheal tube (ETT) inner diameter of >7.5 mm. Pregnant women were excluded. Subjects received all standard supportive care; concomitant use of any other COVID-19 treatments was permitted.

UVA Device

The UVA therapy device consisted of a 5.4 mm diameter sterile sealed multi-LED UVA light catheter within a protective sheath and endotracheal adaptor, umbilical, and control unit. An example UVA therapy device is the UV light treatment assembly described with respect to FIGS. 64-69. The UVA catheter adaptor was connected to the ETT using a double-swivel multi-access port to maintain a closed-loop system and prevent ambient exposure to exhaled air upon introduction of the catheter into the ETT.

Procedure

Within 24 hours of enrollment, subjects underwent 20 minutes of UVA therapy, which was repeated once daily for a total of 5 consecutive days. All subjects received 100% FiO₂ for 30 minutes prior to the procedure. The UVA catheter was inserted into the distal end of the ETT, with concomitant ventilator adjustments to flow rate and tidal volume to maintain optimal oxygenation. A plastic clamp fixed the catheter base to the access port to assure stability and consistent depth of catheter insertion throughout the 20-minute treatment session. Procedural instructional video can be accessed at: Dosing was chosen based on the optimal response of coronavirus 229E infected human primary tracheal cells to UVA exposure observed in in vitro experiments. Controlled UVA emission (peak wavelength 340-345 nm) of maximum 2 milliwatt/cm² was delivered at the level of tracheal mucosa. Predetermined criteria for treatment cessation and withdrawal of the UVA catheter included 02 saturation drop below 88% or hemodynamic instability.

Endotracheal (ET) aspirates were taken prior to each UVA treatment and 24 hours after the last UVA treatment for assessment of SARS-CoV-2 load and total bacterial abundance. Absolute quantification of bacterial load represents culturable and non-culturable, viable and non-viable, pathogenic and non-pathogenic bacteria.

The global challenge associated with COVID-19 pandemic is a bitter reminder that safe and effective therapies are desperately needed to treat resistant and/or novel pathogens. While externally applied UV therapy is commonly used in dermatologic diseases, internal UV therapy has never previously been performed. In this first-in-human study, endotracheal UVA light appeared safe in critically ill patients with COVID-19. Furthermore, a significant reduction in endotracheal SARS-CoV-2 levels was observed following 5 days of UVA therapy.

There is a significant independent association between respiratory SARS-CoV-2 load and mortality. Severe cases of COVID-19 exhibit longer duration and a later peak of virus in respiratory samples as compared to mild disease. Of 5 subjects, 4 had high viral loads in the ET aspirate at baseline, which did not correlate with the time of symptom onset.

No treatment emergent adverse events occurred during the 25 UVA treatment sessions and serious/severe adverse events were unrelated to our intervention. Oxygenation and hemodynamics remained stable during all treatments. Bronchoscopy in 2 subjects revealed normal-looking trachea in line with our preclinical in-vivo and in-vitro safety experiments. Patient Subject number 2 died due to complications of ECMO-related anticoagulation (intracranial hemorrhage) despite stable oxygenation at the time of stroke. Bleeding occurs in approximately 50% of patients undergoing ECMO with intracranial hemorrhage having an 85% risk of mortality. Despite being in a highly critical state, 4 out of 5 subjects survived and had meaningful clinical improvements. (FIG. 73)

Subjects had a diverse distribution of several known risk factors for severity of COVID-19 including age (range 38-65 years), sex (2 females and 3 males), race (1 non-Hispanic white, 3 Hispanic white and 1 African American) and BMI (range 25-36). Of 5 patients, 3 had the smallest allowable ETT size (7.5 mm) without any TEAE; however, patients with ETT<7.5 were not included. The 3.2 log reduction in this study after 5 days of UVA therapy appears to outpace the natural decline of respiratory viral load.

Baseline, hospital and ICU admission-related information including relevant clinical, laboratory, and radiologic data were recorded for all patients until 30 days after enrollment. The World Health Organization (WHO) COVID-19 10-point ordinal severity scale 6 was calculated at enrollment, and on days 15 and 30 following enrollment. SOFA and SAPSIII scores were calculated from the worst values within 24 hours of ICU admission.

Outcomes and Statistical Analysis

The primary endpoint was the change in ET aspirate SARS-CoV-2 viral load from day 0 to the last day of treatment. Secondary outcomes included changes in endotracheal absolute bacterial load and clinical outcomes including length of time on mechanical ventilation, in the ICU, and in the hospital, laboratory parameters including inflammatory markers, and changes in the WHO COVID-19 10-point ordinal scale of improvement from baseline to day 15 and 30.

Freidman test was used to detect differences across daily viral and bacterial loads. One sample t-test was used to analyze changes in inflammatory markers and microbial loads from day 0 to 1. Spearman rank-order test was used to assess correlations. A significance level of α=0.05 was used.

Results:

Five subjects were enrolled (mean age 56.6, 3 male). Baseline characteristics of the enrolled subjects are summarized in FIG. 73 and FIG. 76. At the time of intubation, all 5 patients were critically ill with WHO COVID-19 ordinal scale scores of 9, and with SOFA scores predicting a 20-95% mortality rate. All patients received daily 20-minute treatments starting within the first 36 hours following intubation, for 5 days. Pretreatment and day 6 ET aspirates were taken in all patients except for study subject #1 who was extubated on day 6. Hence, a total of 29 ET aspirates were analyzed.

Primary and Secondary Outcomes

Subjects had elevated viral loads at baseline (range 3.4×10⁴-1.64×10⁷ copies/ml) except for study subject 2 who had undetectable viral load at all-time points demonstrating that virus had cleared since the last nasal swab. There was no significant correlation between symptom onset date and either baseline (Spearman R=−0.70, p=0.23) or day 6 viral loads (Spearman R=−0.21, p=0.83).

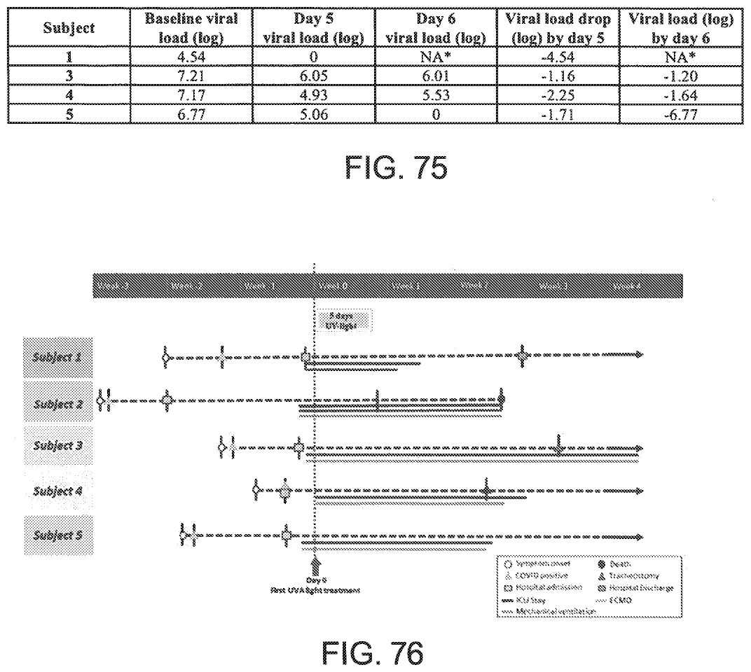

Average log changes in endotracheal viral load from baseline to day 5 and day 6 were −2.41 (range −1.16-−4.54; Friedman p=0.002) and −3.2 (range −1.2-−6.77; Friedman p<0.001), respectively. (FIGS. 74 and 75)

Quantification of absolute endotracheal bacterial load at baseline ranged from 1×10³-1.7×10⁶ CFU/ml and remained statistically unchanged during the UVA treatment sessions. (not shown)

Disease course for each subject is shown in FIG. 76. WHO clinical severity scale improved by an average of 1.6 and 3.6 points on day 15 and day 30, respectively. Excluding subject 2 who had undetectable baseline viral load, WHO severity scale improved by 4.75 points on day 30. (not shown) All subjects survived except study subject 2, who was placed on comfort care following intracranial hemorrhage due to ECMO-associated anticoagulation and died on day 17.

Safety Outcomes

No treatment-emergent adverse event (TEAE) or early treatment discontinuation was observed in the study. Oxygen saturations and hemodynamics during all treatment sessions remained stable. None of the subjects experienced pneumothorax, subcutaneous emphysema or ETT dislodgment. Adverse events were deemed unrelated to UVA therapy. Two subjects underwent bronchoscopy for tracheostomy tube placement for prolonged intubation which revealed normal looking tracheae without erythema or friability. The DSMB did not recommend any changes to the treatment protocol for future planned trials.

The disclosed UV light treatment systems and methods provide many technical advantages. A technical advantage includes significant improvement in the field of internal UV light therapy. Further, the light catheter configuration disclosed herein including a set of LEDs and a cooling tube with an open end within the light catheter allows the UV light catheter to be implemented with a small diameter such that the UV light catheter may be deployed within a ETT or a NPA while providing sufficient cooling of the light catheter. Furthermore, the UVA wavelengths, intensities, and durations disclosed herein provide an effective and safe anti-viral UV therapy. Furthermore, the UV light treatment system configuration provides effective internal treatment against viruses while a patient is being ventilated via a mechanical ventilator.

In one implementation, a UV light delivery device for performing intra-corporeal ultraviolet therapy is provided, the device comprising: an elongated body separated by a proximal end and a distal end, wherein the elongated body comprises at least one receiving space; and at least one UV light source configured to be received at the at least one receiving space, wherein the at least one UV light source is configured to emit a wavelength between 320 nm and 410 with a peak wavelength of 340 nm. In an example of the device, the device may optionally include wherein the at least one UV light source is positioned to emit radiation outwardly from the elongated body. A second example of the device may optionally include the first example and may further include a plurality of UV light sources dispersed along the length of the elongated body. A third example of the device may optionally include one or more of the first and the second examples, and further include a power supply electrically connected to the at least one UV light source. A fourth example of the device may optionally include one or more of the first through third examples, and further include wherein the elongated body comprises four sides. A fifth example of the device may optionally include one or more of the first through fourth examples, and further include wherein each of the four sides of the elongated body comprises at least one receiving space, such that a corresponding plurality of UV light sources are staggered on the elongated body. A sixth example of the device may optionally include one or more of the first through fifth examples, and further include a receiving space and a corresponding UV light source at the proximal end. A seventh example of the device may optionally include one or more of the first through sixth examples, and further include wherein the elongated body is at least partially transparent. An eighth example of the device may optionally include one or more of the first through seventh examples, and further include wherein the elongated body at least partially comprises borosilicate glass. A ninth example of the device may optionally include one or more of the first through eight examples, and further include wherein the elongated body at least partially comprises copper. A tenth example of the device may optionally include one or more of the first through ninth examples, and further include wherein the elongated body comprises a copper body with a borosilicate glass coating. An eleventh example of the device may optionally include one or more of the first through tenth examples, and further include a rotating motor configured to rotate the elongated body and subsequently the at least one UV light source. A twelfth example of the device may optionally include one or more of the first through eleventh examples, and further include a rotating base connected to the distal end of the elongated body such that the elongated body is configured to rotate about the rotating base.

In some embodiments, a method for performing intra-corporeal ultraviolet therapy may comprise providing a UV light delivery device comprising: an elongated body separating a proximal end and a distal end, wherein the elongated body comprises at least two receiving spaces; and at least two UV light sources configured to be received at the at least two receiving spaces; and rotating the elongated body such that the at least two UV light sources are configured to emit UV light outwardly in a uniform manner. A first example of the method may further include emitting, from the at least two UV light sources, a wavelength between 320 nm and 410 with a peak wavelength of 340 nm. A second example of the method optionally includes the first example and further includes emitting, from the at least two UV light sources, radiation outwardly from the elongated body. A third example of the method optionally includes one or more of the first and the second examples, and further includes wherein the elongated body comprises four sides. A fourth example of the method may optionally include one or more of the first through third examples, and may further include wherein each of the four sides of the elongated body comprises at least one receiving space, such that a corresponding plurality of UV light sources are staggered on the elongated body. A fifth example of the method may optionally include one or more of the first through fourth examples, and may further include wherein the elongated body further comprises a receiving space and a corresponding UV light source at the proximal end. A sixth example of the method may optionally include one or more of the first through fifth examples, and may further include wherein the elongated body is at least partially transparent. A seventh example of the method may optionally include one or more of the first through sixth examples, and may further include wherein the elongated body at least partially comprises borosilicate glass. An eighth example of the method may optionally include one or more of the first through seventh examples, and may further include wherein the elongated body at least partially comprises copper.

In another embodiment, a UV light delivery device for performing intra-corporeal ultraviolet therapy may comprise an elongated body separating a proximal end and a distal end, wherein the elongated body comprises at least one receiving space wherein the elongated body and the at least one receiving space comprises a copper body and the elongated body comprises a borosilicate glass coating; and at least one UV light source configured to be received at the at least one receiving space. In a first example, the device may include wherein the at least one UV light source is configured to emit a wavelength between 320 nm and 410 with a peak wavelength in a range from 343 nm-345 nm. In a second example, which may optionally include the first example, the device may include wherein the at least one UV light source is positioned to emit radiation outwardly from the elongated body. In n third example, with optionally include one or more of the first and the second examples, the device may further include a plurality of UV light sources dispersed along the length of the elongated body. In a fourth example of the device, which optionally includes one or more of the first through the third examples, the device further includes a power supply electrically connected to the at least one UV light source. In a fifth example of the device, which optionally includes one or more of the first through the fourth examples, the device further includes wherein the elongated body comprises four sides. In a sixth example of the device, which optionally includes one or more of the first through the fifth examples, the device further includes wherein the at least one UV light source comprises a light emitting diode. In a seventh example of the device, which optionally includes one or more of the first through the sixth examples, the device further includes wherein each of the four sides of the elongated body comprises at least one receiving space, such that a corresponding plurality of UV light sources are staggered on the elongated body. In an eighth example of the device, which optionally includes one or more of the first through the seventh examples, the device further includes a corresponding UV light source at the proximal end. In a tenth example of the device, which optionally includes one or more of the first through the ninth examples, the device further includes a rotating motor configured to rotate the elongated body and subsequently the at least one UV light source. In an eleventh example of the device, which optionally includes one or more of the first through the tenth examples, the device further includes a rotating base connected to the distal end of the elongated body such that the elongated body is configured to rotate about the rotating base.

In another embodiment, a method of performing an antimicrobial therapy on a patient comprises radiating a patient's internal tissue with a light source emitting a set of wavelengths in the UV-A and/or UV-B range for at least 10 minutes. A first example of the method may further include wherein then UV-A and/or UV-B range comprises at least 320-345 nm. A second example of the method may optionally include the first example, and further include wherein at least 10 minutes comprises 18-22 minutes. A third example of the method may optionally include one or more of the first and the second examples, and may further include wherein the intensity of application is 2,000 microwatt/cm² and a distance to the patient's internal tissue comprises 0-1 cm.

### Selected Embodiments

Although the above description and the attached claims disclose a number of embodiments of the present invention, other alternative aspects of the invention are disclosed in the following further embodiments.

Embodiment 1. A system for performing intra-corporeal ultraviolet therapy, the system comprising: an endotracheal tube (ETT); and a light catheter comprising: a light delivery portion comprising a set of LEDs positioned to emit light circumferentially outward; a cooling tube comprising at least one opening; and an ETT connector configured to connect to the ETT.
Embodiment 2. The system of embodiment 1, wherein a portion of each LED in the set of LEDs is in direct contact with the cooling tube.
Embodiment 3. The system of embodiment 1, wherein within the cooling tube, a coolant gas flows in a first direction towards the at least one opening and exits via the at least one opening, and flows backwards within the light catheter in a second direction opposite to the first direction.
Embodiment 4. The system of embodiment 1, further comprising a heat sink coupled to each LED in the set of LEDs.
Embodiment 5. The system of embodiment 1, wherein the set of LEDs emit peak wavelengths in the 340-349 nm range.
Embodiment 6. The system of embodiment 1, wherein the set of LEDs emit wavelengths between 320 nm and 410 with a peak wavelength in a range from 343 nm to 345 nm.
Embodiment 7. The system of embodiment 1, wherein the set of LEDs emit peak wavelengths in the 340-345 nm range.
Embodiment 8. The system of embodiment 1, wherein the ETT connector comprises a flap valve.
Embodiment 9. The system of embodiment 1, further comprising a compressor system comprising: one or more processors; an air compressor; and a dual connector comprising an air connector and an electrical connector.
Embodiment 10. The system of embodiment 9, further comprising an umbilical tube comprising: an air passageway; electrical conductors; a light catheter connector configured to connect to the light catheter; and a compressor connector configured to connect to the compressor system.
Embodiment 11. The system of embodiment 10, further comprising a light source controller comprising: one or more processors; a memory; a control system coupled to the memory comprising one or more processors, the control system configured to execute machine executable code to cause the set of LEDs to emit light for a specified duration and an intensity.
Embodiment 12. The system of embodiment 11, wherein the specified duration is at least 20 minutes, 40 minutes, or 60 minutes daily, for at least one, two, three, four or five days.
Embodiment 13. The system of embodiment 11, wherein the intensity comprises at least 1,100 microwatt/cm², 1,500 microwatt/cm², 2,000 microwatt/cm², 2,100 microwatt/cm², 2,200 microwatt/cm², 2,300 microwatt/cm², 2,400 microwatt/cm², 2,500 microwatt/cm², 2,600 microwatt/cm², 2,700 microwatt/cm², 2,800 microwatt/cm², 2,900 microwatt/cm², 3,000 microwatt/cm², or 2 milliwatt/cm².
Embodiment 14. A method of deploying the light catheter in the system for performing intra-corporeal ultraviolet therapy of embodiment 11, the method comprising: connecting the ETT connector to the ETT; deploying the light catheter into the ETT by advancing the light catheter through the flap valve; providing instructions to the controller to energize the set of LEDs; and energizing the air compressor to pump air through the air passageway into the cooling tube and out of the at least one opening.
Embodiment 15. The method of embodiment 14, further comprising determining a temperature based on signals received from a thermistor in thermal contact with the light delivery portion and adjusting the flow rate of the air compressor based on the determined temperature.
Embodiment 16. The method of embodiment 14, further comprising determining a temperature based on signals received from a thermistor in thermal contact with the light delivery portion and adjusting the power to the LEDs delivered by the light source controller based on the determined temperature.
Embodiment 17. A method of treating a patient with a respiratory infection, the method comprising: intubating the patient with an ETT; connecting a light catheter to the ETT, wherein the light catheter comprises a set of LEDs and a cooling channel; radiating UV-A light outwardly from the light catheter along a substantial length of the light catheter from the set of LEDs to treat an infection in the patient while ventilating the patient.
Embodiment 18. The method of embodiment 17, wherein the infection comprises at least one of pneumonia, a bacteria, a virus, an RNA virus, a coronavirus, or SARS-CoV-2.
Embodiment 19. The method of embodiment 17, wherein the radiating is performed for 20 minutes at 2,000 microWatt/cm² intensity.
Embodiment 20. The method of embodiment 17, wherein the radiating is performed using at least 1,000 microWatt/cm² intensity.
Embodiment 21. The method of embodiment 17, wherein the infection is SARS-CoV-2 and the radiating is performed for at least 20 minutes daily, for at least five days.
Embodiment 22. The method of embodiment 17, wherein radiating is performed for at least 10 minutes and between 1,000-5,000 microWatt/cm² intensity.
Embodiment 23. The method of embodiment 17, wherein radiating the light outwardly from the ETT is performed using a UV light source integrated in a catheter, introduced inside a canal in the ETT.
Embodiment 24. A method of treating a patient with a respiratory infection, the method comprising: intubating the patient with an ETT; and radiating UV-A light outwardly from the ETT to treat an infection.
Embodiment 25. The method of embodiment 24, wherein the infection comprises at least one of pneumonia, a bacteria, a virus, an RNA virus, or a coronavirus.
Embodiment 26. The method of embodiment 24, wherein the radiating is performed for between 10-30 minutes at between 1,000-5,000 microWatt/cm² intensity.
Embodiment 27. The method of embodiment 24, wherein the radiating is performed using at least 1,000 microWatt/cm² intensity.
Embodiment 28. The method of embodiment 24, wherein the radiating is performed for at least 10 minutes and between 1,000-5,000 microWatt/cm² intensity.
Embodiment 29. The method of embodiment 24, wherein the radiating is performed using at least 2,000 microWatt/cm² intensity.
Embodiment 30. The method of embodiment 24, wherein the radiating is performed for 18-22 minutes.
Embodiment 31. The method of embodiment 24, wherein radiating the light outwardly from the ETT is performed using a UV light source separate from the ETT.
Embodiment 32. The method of embodiment 24, wherein radiating the light outwardly from the ETT is performed using a UV light source integrated with the ETT.
Embodiment 33. A system for performing intra-corporeal ultraviolet therapy, the device comprising: an endotracheal tube (ETT); and a UV light delivering device configured to emit light through a portion of the ETT.
Embodiment 34. The system of embodiment 33, wherein the UV light delivering device is separate from the ETT.
Embodiment 35. The system of embodiment 33, wherein the UV light delivering device is a configured to connect to the ETT.
Embodiment 36. The system of embodiment 33, wherein the UV light delivering device is a configured to fit inside the ETT.
Embodiment 37. A method for performing intra-corporeal ultraviolet therapy comprising: providing a UV light delivery device comprising a UV light source; and inserting the UV light delivery device through a nasal canal of the patient to treat an infection.
Embodiment 38. The method of embodiment 37, wherein the UV light sources comprises at least one LED configured to emit peak wavelengths in the 340-349 nm range.
Embodiment 39. The method of embodiment 37, wherein the infection is a coronavirus infection, or other RNA virus infection.
Embodiment 40. The method of embodiment 37, wherein the UV light source is navigated to a respiratory canal.
Embodiment 41. The method of embodiment 37, wherein the UV light source is a UV-A light source.
Embodiment 42. The method of embodiment 37, wherein the UV light source is activated for between 10-30 minutes.

### CONCLUSION

The various methods and techniques described above provide a number of ways to carry out the invention. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

In some embodiments, the terms “a” and “an” and “the” and similar references used in the context of describing a particular embodiment of the application (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application.

Certain embodiments of this application are described herein. Variations on those embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

Particular implementations of the subject matter have been described. Other implementations are within the scope of the following claims. In some cases, the actions recited in the claims can be performed in a different order and still achieve desirable results. In addition, the processes depicted in the accompanying figures do not necessarily require the particular order shown, or sequential order, to achieve desirable results.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

1. A system for performing intra-corporeal ultraviolet therapy, the system comprising:
an endotracheal tube (ETT); and
a light catheter comprising:
a light delivery portion comprising a set of LEDs positioned to emit light circumferentially outward;
a cooling tube comprising at least one opening; and
an ETT connector configured to connect to the ETT.

2. The system of claim 1, wherein a portion of each LED in the set of LEDs is in direct contact with the cooling tube.

3. The system of claim 1, wherein within the cooling tube, a coolant gas flows in a first direction towards the at least one opening and exits via the at least one opening, and flows backwards within the light catheter in a second direction opposite to the first direction.

4. The system of claim 1, further comprising a heat sink coupled to each LED in the set of LEDs.

5. The system of claim 1, wherein the set of LEDs emit peak wavelengths in the 340-349 nm range.

6. The system of claim 1, wherein the set of LEDs emit wavelengths between 320 nm and 410 with a peak wavelength in a range from 343 nm to 345 nm.

7. The system of claim 1, wherein the set of LEDs emit peak wavelengths in the 340-345 nm range.

8. The system of claim 1, wherein the ETT connector comprises a flap valve.

9. The system of claim 1, further comprising a compressor system comprising:
one or more processors;
an air compressor; and
a dual connector comprising an air connector and an electrical connector.

10. The system of claim 9, further comprising an umbilical tube comprising:
an air passageway;
electrical conductors;
a light catheter connector configured to connect to the light catheter; and
a compressor connector configured to connect to the compressor system.

11. The system of claim 10, further comprising a light source controller comprising:
one or more processors;
a memory;
a control system coupled to the memory comprising one or more processors, the control system configured to execute machine executable code to cause the set of LEDs to emit light for a specified duration and an intensity.

12. The system of claim 11, wherein the specified duration is at least 20 minutes, 40 minutes, or 60 minutes daily, for at least one, two, three, four or five days.

13. The system of claim 11, wherein the intensity comprises at least 1,100 microwatt/cm², 1,500 microwatt/cm², 2,000 microwatt/cm², 2,100 microwatt/cm², 2,200 microwatt/cm², 2,300 microwatt/cm², 2,400 microwatt/cm², 2,500 microwatt/cm², 2,600 microwatt/cm², 2,700 microwatt/cm², 2,800 microwatt/cm², 2,900 microwatt/cm², 3,000 microwatt/cm², or 2 milliwatt/cm².

14. A method of deploying the light catheter in the system for performing intra-corporeal ultraviolet therapy of claim 11, the method comprising:
connecting the ETT connector to the ETT;
deploying the light catheter into the ETT by advancing the light catheter through the flap valve;
providing instructions to the controller to energize the set of LEDs; and
energizing the air compressor to pump air through the air passageway into the cooling tube and out of the at least one opening.

15. The method of claim 14, further comprising determining a temperature based on signals received from a thermistor in thermal contact with the light delivery portion and adjusting the flow rate of the air compressor based on the determined temperature.

16. The method of claim 14, further comprising determining a temperature based on signals received from a thermistor in thermal contact with the light delivery portion and adjusting the power to the LEDs delivered by the light source controller based on the determined temperature.

17. A method of treating a patient with a respiratory infection, the method comprising:
intubating the patient with an ETT;
connecting a light catheter to the ETT, wherein the light catheter comprises a set of LEDs and a cooling channel;
radiating UV-A light outwardly from the light catheter along a substantial length of the light catheter from the set of LEDs to treat an infection in the patient while ventilating the patient.

18. The method of claim 17, wherein the infection comprises at least one of pneumonia, a bacteria, a virus, an RNA virus, a coronavirus, or SARS-CoV-2.

19. The method of claim 17, wherein the radiating is performed for 20 minutes at 2,000 microWatt/cm² intensity.

20. The method of claim 17, wherein the radiating is performed using at least 1,000 microWatt/cm² intensity.

21. The method of claim 17, wherein the infection is SARS-CoV-2 and the radiating is performed for at least 20 minutes daily, for at least five days.

22. The method of claim 17, wherein radiating is performed for at least 10 minutes and between 1,000-5,000 microWatt/cm² intensity.

23. The method of claim 17, wherein radiating the light outwardly from the ETT is performed using a UV light source integrated in a catheter, introduced inside a canal in the ETT.
